(19) United States
(12) Patent Application Publication
(10) Pub. No.: US 2026/0347318 A1
(43) Pub. Date: Nov. 5, 2026

(54) ONIUM SALT, PHOTOACID GENERATOR, CHEMICALLY-AMPLIFIED RESIST COMPOSITION, AND PATTERNING PROCESS

(71) Applicant: SHIN-ETSU CHEMICAL CO., LTD., Tokyo (JP)

(72) Inventors: Ryo SUDO, Joetsu-shi (JP); Miyu YOSHIDA, Joetsu-shi (JP)

(73) Assignee: SHIN-ETSU CHEMICAL CO., LTD., Tokyo (JP)

(21) Appl. No.: 19/638,494

(22) Filed: Apr. 3, 2026

(30) Foreign Application Priority Data
Apr. 16, 2025 (JP) 2025- 067571

(51) Int. Cl. C07C 381/12; G03F 7/004; G03F 7/20

(52) U.S. Cl. CPC C07C 381/12; G03F 7/0045; G03F 7/2004

(57) ABSTRACT

The present invention is an onium salt of formula (a). This can provide a chemically-amplified resist composition and a patterning process that yield high sensitivity, favorable CDU, favorable MEF, and a favorable pattern profile after exposure.

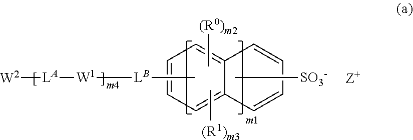

R⁰ represents an electron-withdrawing group other than a fluorine atom; R¹ represents a fluorine atom, a hydroxy group, or a hydrocarbyl group having 1-20 carbon atoms and optionally having a heteroatom; L^{A} and L^{B} represent a single bond, an ether bond, an ester bond, an amide bond, a sulfonate ester bond, a sulfonamide bond, a carbonate bond, or a carbamate bond; W¹ represents a hydrocarbylene group having 1-40 carbon atoms, optionally having a heteroatom, and not involving resonance structures; W² represents a hydrocarbyl group having 1-40 carbon atoms, optionally having a heteroatom, and not involving resonance structures; and Z⁺ represents a sulfonium cation of formula (Z-1) or an iodonium cation of formula (Z-2).

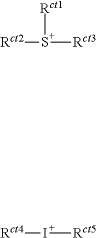

### TECHNICAL FIELD

The present invention relates to: an onium salt; a photoacid generator; a chemically-amplified resist composition; and a patterning process.

### BACKGROUND ART

In recent years, as LSIs advance toward higher integration and higher processing speed, there has been a demand for fine pattern formation, and for pattern processing at 0.2 μm or less, chemically-amplified resist compositions using an acid catalyst have been used. In these cases, high-energy radiations, such as ultraviolet rays, deep ultraviolet rays, and electron beams, are used as an exposure source. As one of the most advanced miniaturization technologies, mass production using ArF immersion lithography, in which exposure is performed by inserting a liquid, such as water, between a projection lens and a substrate, is implemented. And studies on multiple exposures of ArF lithography and on extreme ultraviolet (EUV) lithography with a wavelength of 13.5 nm are under investigation.

For the chemically-amplified resist compositions used in the above-described lithography, a compound (hereinafter, referred to as “photoacid generator”) that is decomposed upon exposure to light to generate an acid is used, and by optimizing a structure in the photoacid generator, acid diffusion can be suppressed, and it is possible to form a high-resolution pattern. Generally, by shortening the length of the acid diffusion, image blurs due to acid diffusion are eliminated, and favorable mask CD dependence (Mask Error Factor: MEF) and favorable critical dimension uniformity (CDU) are exhibited.

Meanwhile, in the ArF immersion lithography, since a light source having a wavelength of 193 nm is used, it is most common to select a photoacid generator that is highly transparent at a wavelength of 193 nm. Therefore, by incorporating an alicyclic structure, such as adamantane and norbornane, into an acid generator, the acid diffusion can be suppressed effectively. Furthermore, structures of adamantanone or norbornanelactone, in which a heteroatom, such as an oxygen atom, is incorporated into adamantane or norbornane, can further suppress acid diffusion. Patent Document 1 proposes an acid generator having a particular alicyclic structure.

Conventional photoacid generators have enhanced acidity by having an electron-withdrawing group, such as a halogen atom, near an acid-generating moiety. Especially, a fluorine atom has the highest electronegativity, and therefore, onium salts having a perfluoroalkanesulfonate anion are generally used as a photoacid generator. However, perfluoroalkanesulfonic acid, which is the acid to be generated, does not have a decomposable group, such as an ester structure, and is thereby hardly decomposed in the natural world; therefore, it is a hardly decomposable compound, which persists in the environment for a long time. Moreover, the perfluoroalkanesulfonic acid is water-soluble; therefore, it diffuses widely through river systems, has significant environmental load, and is regarded as a problem in modern society.

Patent Document 2 discloses resist compositions containing an acid generator that generates fluorobenzenesulfonic acid, which are useful as acid generators with relatively low environmental load. Furthermore, because a bulky alicyclic structure can be incorporated between terminally iodinated aromatic ring group and fluorobenzenesulfonic acid, they are also useful as a low-diffusion photoacid generator. On the other hand, aromatic cyclic groups are unsuitable because transmittance is reduced, thereby reducing exposure sensitivity, when used in resist compositions for ArF immersion lithography. Electron-withdrawing groups can increase the acid strength of sulfonic acids. A fluorine atom is also one of the electron-withdrawing groups, but when an aryl hydrogen of benzenesulfonic acid is substituted with a fluorine atom, satisfactory acid strength could not be achieved due to the resonance effect of the aromatic group.

### CITATION LIST

### Patent Literature

Patent Document 1: JP2015-117200A
Patent Document 2: JP2018-159744A

### SUMMARY OF INVENTION

### Technical Problem

In this way, the prior art has a problem in that it has not provided a resist composition and a patterning process that simultaneously satisfy lithography performance, such as sensitivity, CDU, and MEF, and also has low environmental load, in device mass production using ArF immersion lithography.

The present invention has been made in view of the above-described problem. An object of the present invention is to provide a chemically-amplified resist composition and a patterning process that yields higher sensitivity than the conventional resist compositions, favorable CDU, favorable MEF, and a favorable pattern profile after exposure to light.

### Solution to Problem

To solve the above-described problem, the present invention provides
an onium salt represented by the following general formula (a),

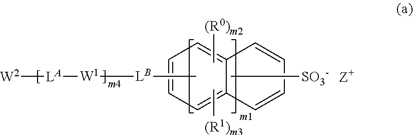

wherein
“m1” represents 0 to 1; “m2” represents an integer of 1 to 4 when “m1” is 0, “m2” represents an integer of 1 to 6 when “m1” is 1, “m3” represents an integer of 0 to 3 when “m1” is 0, “m3” represents an integer of 0 to 5 when “m1” is 1, provided that 2≤m2+m3≤4 when “m1” is 0 and 2≤m2+m3≤6 when “m1” is 1; and “m4” represents an integer of 0 to 2;
R⁰ represents an electron-withdrawing group other than a fluorine atom, and R⁰s may be the same as or different from each other when “m2” is 2 or more;
R¹ represents a fluorine atom, a hydroxy group, or a hydrocarbyl group having 1 to 20 carbon atoms and optionally having a heteroatom, R¹s may be the same as or different from each other when “m3” is 2 or more, and furthermore multiple R¹s may be bonded to each other to form a ring together with the carbon atoms to which they are bonded when “m3” is 2 or more;
L^{A} and L^{B} each independently represent a single bond, an ether bond, an ester bond, an amide bond, a sulfonate ester bond, a sulfonamide bond, a carbonate bond, or a carbamate bond;
W¹ represents a hydrocarbylene group having 1 to 40 carbon atoms, optionally having a heteroatom, and not involving resonance structures; W² represents a hydrocarbyl group having 1 to 40 carbon atoms, optionally having a heteroatom, and not involving resonance structures; and
Z⁺ represents a sulfonium cation represented by the following general formula (Z-1) or an iodonium cation represented by the following general formula (Z-2),

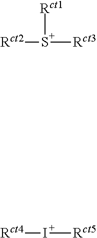

wherein
R^{ct1} to R^{ct5} each independently represent a halogen atom or a hydrocarbyl group having 1 to 30 carbon atoms and optionally having a heteroatom, and R^{ct1} and RCt² may be bonded to each other to form a ring together with the sulfur atom to which they are bonded.

By using a chemically-amplified resist composition containing such an onium salt, it is possible to obtain a resist pattern that exhibits high sensitivity, favorable CDU, favorable MEF, and an excellent pattern profile.

At least one of W¹ and W² in the general formula (a) have preferably an alicyclic structure having 3 to 40 carbon atoms and optionally having a hetero atom.

Such an onium salt can effectively suppress acid diffusion when used as a photoacid generator.

The onium salt of the present invention is preferably represented by the following general formula (a1),

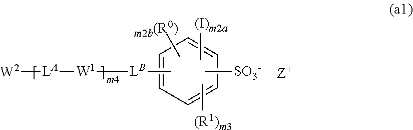

wherein “m3”, “m4”, L^{A}, L^{B}, R⁰, R¹, W¹, W², and Z⁺ represent the same as defined above, “m2a” represents an integer of 1 to 4, and “m2b” represents an integer of 0 to 3.

In such an onium salt, the benzene ring to which the sulfonate anion is bonded is substituted with an iodine atom, and the electron-withdrawing property thereof increases the acidity, and high sensitivity can be expected.

The onium salt of the present invention is preferably represented by the following general formula (a2),

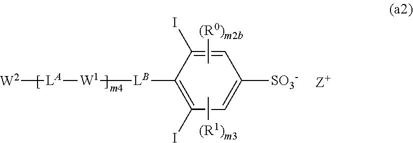

wherein “m4”, L^{A}, L^{B}, R⁰, R¹, W¹, W², and Z⁺ represent the same as defined above, and “m2b” and “m3” represent an integer of 0 to 2.

Such onium salts have a structure in which an iodine atom is bonded to an aromatic ring, and are therefore expected to be a material with lower environmental load than fluoroalkanesulfonic acids.

Furthermore, the present invention provides a photoacid generator comprising the above-mentioned onium salt.

The onium salt of the present invention can be suitably used as a photoacid generator.

Furthermore, the present invention provides a chemically-amplified resist composition comprising the photoacid generator (A) described above.

A chemically-amplified resist composition containing such a photoacid generator exhibits high sensitivity and can form a pattern having improved CDU and improved MEF.

In the present invention, the chemically-amplified resist composition preferably further comprises a base polymer having a repeating unit having an acid-labile group (B).

The chemically-amplified resist composition of the present invention may contain such a component (B).

In the present invention, the repeating unit having an acid-labile group is preferably represented by the following general formula (B1),

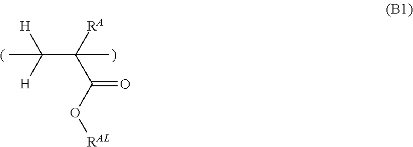

wherein R^{A} represents a hydrogen atom or a methyl group, and R^{AL} represents an acid-labile group.

The chemically-amplified resist composition of the present invention may contain such a component (B1).

In the chemically-amplified resist composition of the present invention, the base polymer has at least one selected from the group consisting of a repeating unit represented by the following general formula (B2), a repeating unit represented by the following general formula (B3), and a repeating unit represented by the following general formula (B4),

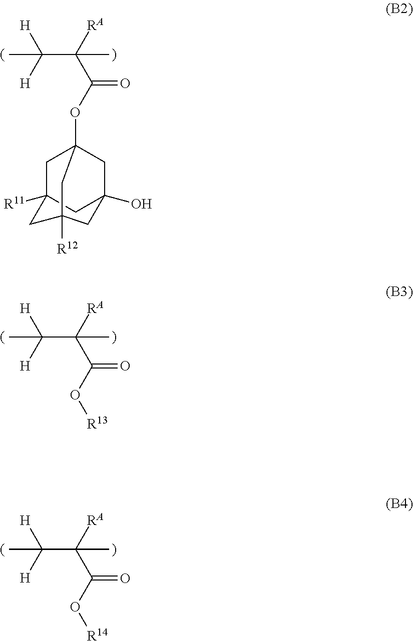

wherein
R^{A} represents a hydrogen atom or a methyl group;
R¹¹ and R¹² each independently represent a hydrogen atom or a hydroxy group;
R¹³ represents a substituent having a lactone structure or a substituent having a sultone structure; and
R¹⁴ represents a hydrogen atom, a fluorinated hydrocarbyl group having 1 to 15 carbon atoms, or a monovalent fluoroalcohol-containing substituent having 1 to 15 carbon atoms.

The chemically-amplified resist composition of the present invention may contain such a base polymer.

The chemically-amplified resist composition of the present invention may further comprise an organic solvent (C).

The chemically-amplified resist composition of the present invention may contain such a component (C).

The chemically-amplified resist composition may further comprise a fluorine-atom-containing polymer (D) having at least one selected from the group consisting of a repeating unit represented by the following general formula (D1), a repeating unit represented by the following general formula (D2), a repeating unit represented by the following general formula (D3), and a repeating unit represented by the following general formula (D4),

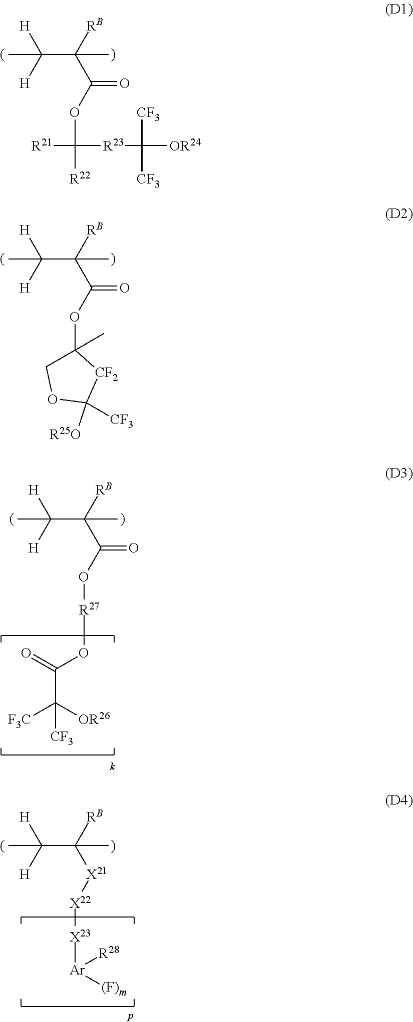

wherein
R^{B} represents a hydrogen atom or a methyl group;
R²¹ and R²² each independently represent a hydrogen atom or a hydrocarbyl group having 1 to 10 carbon atoms;
R²³ represents a single bond or a linear or branched hydrocarbylene group having 1 to 5 carbon atoms;
R²⁴, R²⁵, and R²⁶ each independently represent a hydrogen atom, a hydrocarbyl group having 1 to 15 carbon atoms, a fluorinated hydrocarbyl group having 1 to 15 carbon atoms, an acyl group having 2 to 15 carbon atoms, or an acid-labile group, when R²⁴, R²⁵, and R²⁶ are a hydrocarbyl group or a fluorinated hydrocarbyl group, part of —CH₂— thereof may be substituted with an ether bond or a carbonyl group;
R²⁷ represents a (k+1)-valent hydrocarbon group having 1 to 20 carbon atoms or a (k+1)-valent fluorinated hydrocarbon group having 1 to 20 carbon atoms;
“k” represents an integer of 1 to 3;
X²¹ each independently represents a single bond, an ester bond, an ether bond, an amide bond, or a phenylene bond;
“p” represents an integer of 1 or 2;
X²² each independently represents a single bond or a hydrocarbylene group having 1 to 20 carbon atoms when “p” is 1, X²² each independently represents a trivalent hydrocarbon group having 1 to 20 carbon atoms when “p” is 2, and the hydrocarbylene group and the trivalent hydrocarbon group may have at least one selected from an oxygen atom, a sulfur atom, a nitrogen atom, and a halogen atom;
X²³ each independently represents a single bond, an ester bond, an ether bond, an amide bond, a sulfonic acid ester bond, a urethane bond, a thiourethane bond, or a urea bond; Ar represents a group derived from benzene or naphthalene;
R²⁸ each independently represents a hydrogen atom, a saturated hydrocarbyl group having 1 to 6 carbon atoms, a saturated hydrocarbyloxy group having 1 to 6 carbon atoms, a saturated hydrocarbyloxycarbonyl group having 2 to 6 carbon atoms, a hydroxy group, a carboxy group, a halogen atom, a cyano group, or a nitro group; and
“m” represents an integer of 1 to 5.

The chemically-amplified resist composition of the present invention may contain such a component (D).

The chemically-amplified resist composition of the present invention may further comprise a quencher (E).

By including the quencher (E) in the chemically-amplified resist composition of the present invention, the acid generated from the photoacid generator can be trapped to prevent the acid from diffusing into unexposed areas.

The chemically-amplified resist composition of the present invention may further comprise a surfactant (G).

The chemically-amplified resist composition of the present invention may contain such a component (G).

Furthermore, the present invention provides a patterning process comprising the steps of: forming a resist film on a substrate by using the chemically-amplified resist composition of the present invention; exposing the resist film by using a high-energy radiation; and developing the exposed resist film by using a developer.

By using the patterning process of the present invention, it is possible to obtain a resist pattern that exhibits high sensitivity, favorable CDU, favorable MEF, and an excellent pattern profile.

In the inventive patterning process, KrF excimer laser light, ArF excimer laser light, an electron beam, or an extreme ultraviolet ray having a wavelength of 3 to 15 nm may be used as the high-energy radiation.

Such high-energy radiation may be used in the patterning process of the present invention.

### Advantageous Effects of Invention

As described above, a chemically-amplified resist composition containing the inventive onium salt as a photoacid generator exhibits high sensitivity and can form a pattern with improved CDU and improved MEF. Furthermore, the inventive chemically-amplified resist composition is suitable as a material for ArF immersion lithography and is useful for both positive pattern formation by alkaline development and negative pattern formation by organic solvent development. Furthermore, the inventive patterning process can be applied to mass production of 5-nm node devices, which requires fine patterns that correspond to higher integration and higher processing speed of LSIs, as well as to the mass production of next-generation 3-nm node devices and generation after next-generation 2-nm node devices.

### DESCRIPTION OF EMBODIMENTS

As described above, it has been desired to develop a chemically-amplified resist composition and a patterning process that yield high sensitivity, favorable CDU, favorable MEF, and a favorable pattern profile after exposure to light.

As a result of their diligent study of the above problems, the inventors found that using a chemically-amplified resist composition containing an aromatic sulfonate substituted with an electron-withdrawing group as a photoacid generator makes it possible to obtain a resist pattern that exhibits high sensitivity, favorable CDU, favorable MEF, and an excellent pattern profile, and have completed the present invention.

That is, the present invention is an onium salt represented by the following general formula (a),

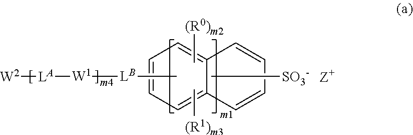

wherein
“m1” represents 0 to 1; “m2” represents an integer of 1 to 4 when “m1” is 0, “m2” represents an integer of 1 to 6 when “m1” is 1, “m3” represents an integer of 0 to 3 when “m1” is 0, “m3” represents an integer of 0 to 5 when “m1” is 1, provided that 2≤m2+m3≤4 when “m1” is 0 and 2≤m2+m3≤6 when “m1” is 1; and “m4” represents an integer of 0 to 2;
R⁰ represents an electron-withdrawing group other than a fluorine atom, and R⁰s may be the same as or different from each other when “m2” is 2 or more;
R¹ represents a fluorine atom, a hydroxy group, or a hydrocarbyl group having 1 to 20 carbon atoms and optionally having a heteroatom, R¹s may be the same as or different from each other when “m3” is 2 or more, and furthermore multiple R¹s may be bonded to each other to form a ring together with the carbon atoms to which they are bonded when “m3” is 2 or more;
L^{A} and L^{B} each independently represent a single bond, an ether bond, an ester bond, an amide bond, a sulfonate ester bond, a sulfonamide bond, a carbonate bond, or a carbamate bond;
W¹ represents a hydrocarbylene group having 1 to 40 carbon atoms, optionally having a heteroatom, and not involving resonance structures; W² represents a hydrocarbyl group having 1 to 40 carbon atoms, optionally having a heteroatom, and not involving resonance structures; and
Z⁺ represents a sulfonium cation represented by the following general formula (Z-1) or an iodonium cation represented by the following general formula (Z-2),

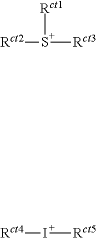

wherein
R^{ct1} to R^{ct5} each independently represent a halogen atom or a hydrocarbyl group having 1 to 30 carbon atoms and optionally having a heteroatom, and R^{ct1} and R^{ct2} may be bonded to each other to form a ring together with the sulfur atom to which they are bonded.

The present invention will be described in detail below, but is not limited thereto. It should be understood that some structures represented by a chemical formula may have an asymmetric carbon, which allows for the existence of enantiomers and diastereomers; in such cases, a single formula represents all of these isomers. One kind of those isomers may be used, or two or more kinds thereof may be used in mixture.

### [Onium Salt]

The onium salt of the present invention is represented by the following general formula (a).

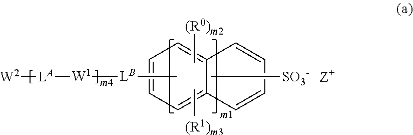

In the general formula,
“m1” represents 0 to 1; “m2” represents an integer of 1 to 4 when “m1” is 0, “m2” represents an integer of 1 to 6 when “m1” is 1, “m3” represents an integer of 0 to 3 when “m1” is 0, “m3” represents an integer of 0 to 5 when “m1” is 1, provided that 2≤m2+m3≤4 when “m1” is 0 and 2≤m2+m3≤6 when “m1” is 1; and “m4” represents an integer of 0 to 2;
R⁰ represents an electron-withdrawing group other than a fluorine atom, and R⁰s may be the same as or different from each other when “m2” is 2 or more;
R¹ represents a fluorine atom, a hydroxy group, or a hydrocarbyl group having 1 to 20 carbon atoms and optionally having a heteroatom, R¹s may be the same as or different from each other when “m3” is 2 or more, and furthermore multiple R¹s may be bonded to each other to form a ring together with the carbon atoms to which they are bonded when “m3” is 2 or more;
L^{A} and L^{B} each independently represent a single bond, an ether bond, an ester bond, an amide bond, a sulfonate ester bond, a sulfonamide bond, a carbonate bond, or a carbamate bond;
W¹ represents a hydrocarbylene group having 1 to 40 carbon atoms, optionally having a heteroatom, and not involving resonance structures; W² represents a hydrocarbyl group having 1 to 40 carbon atoms, optionally having a heteroatom, and not involving resonance structures; and
Z⁺ represents a sulfonium cation represented by the following general formula (Z-1) or an iodonium cation represented by the following general formula (Z-2).

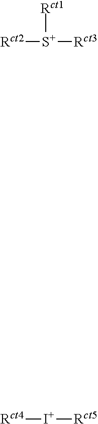

In the formula,
R^{ct1} to R^{ct5} each independently represent a halogen atom or a hydrocarbyl group having 1 to 30 carbon atoms and optionally having a heteroatom, and R^{ct1} and R^{ct2} may be bonded to each other to form a ring together with the sulfur atom to which they are bonded.

In the general formula (a), “m1” represents 0 or 1. When “m1” is 0, it is a benzene ring, and when “m1” is 1, it is a naphthalene ring. In the case of a benzene ring, solvent solubility is ensured, and in the case of a naphthalene ring, the flexibility of the sulfonic acid is controlled, and it is expected that acid diffusion is further suppressed. “m2” represents an integer of 1 to 4 when “m1” is 0, and an integer of 1 to 6 when “m1” is 1. “m3” represents an integer of 0 to 3 when “m1” is 0, and an integer of 0 to 5 when “m1” is 1. However, it is provided that 2≤m2+m3≤4 when “m1” is 0 and 2≤m2+m3≤6 when “m1” is 1. “m4” represents an integer of 0 to 2. When “m4” is 1 or 2, it is preferable because further suppression of acid diffusion is expected.

In the general formula (a), R⁰ is an electron-withdrawing group other than a fluorine atom. When “m2” is 2 or greater, R⁰s may be the same as or different from each other. The electron-withdrawing group represented by R⁰ preferably has a m-position substituent constant om, defined in the Hammett rule described in Chem. Rev. 1991, 91, 165-169, of greater than 0, more preferably greater than 0.35, and is further preferably an iodine atom, a nitro group, a cyano group, an acetyl group, an ester group, or a carbonyl group. A halogen atom or an electron-withdrawing group can enhance the acid strength of the sulfonic acid by an inductive effect caused by their bonding to a carbon atom on the aromatic ring of a benzenesulfonic acid. Although a fluorine atom is the most electronegative, it has a stronger ability to activate the benzene ring through the resonance effects than other electron-withdrawing groups, resulting in a lower acid strength than when substituted with other halogen atoms, such as a chlorine atom, a bromine atom, and an iodine atom. Furthermore, any of the aforementioned electron-withdrawing groups have a higher atomic weight than a fluorine atom, making the benzenesulfonic acid structure rigid and thus useful for suppressing acid diffusion. In particular, when R¹ has a hydrogen bond donor, such as a carboxylic acid, alcohol, or aldehyde, it is preferable that R¹ is present at the m-position or the p-position relative to the sulfonate anion. If the hydrogen bond donor is present at the o-position relative to the sulfonate anion, the generated acid is unevenly distributed due to intramolecular hydrogen bonding, resulting in reduced sensitivity. The Hammett's substituent constant om for each group in this specification is taken from the description in Chem. Rev. 1991, 91, 165-169. But when om is not listed, the om can be determined by calculating the difference between the pKa of benzoic acid and the pKa of a benzoic acid derivative having a substituent at the m-position, by using the software ACD/ChemSketch (ACD/Labs 8.00 Release Product Version: 8.08).

In the above general formula (a), R¹ represents a fluorine atom, a hydroxy group, or a hydrocarbyl group having 1 to 20 carbon atoms and optionally having a heteroatom, R¹s may be the same as or different from each other when “m3” is 2 or more, and multiple R¹s may be bonded to each other to form a ring together with the carbon atoms to which they are bonded when “m3” is 2 or more. Specific examples include a fluorine atom; an alkyl group having 1 to 20 carbon atoms, such as a methyl group, an ethyl group, an n-propyl group, an isopropyl group, an n-butyl group, an isobutyl group, a sec-butyl group, a tert-butyl group, an n-pentyl group, an n-hexyl group, an n-octyl group, an n-nonyl group, an n-decyl group, an undecyl group, a dodecyl group, a tridecyl group, a tetradecyl group, a pentadecyl group, a heptadecyl group, an octadecyl group, a nonadecyl group, and an icosyl group; saturated cyclic hydrocarbyl groups having 3 to 20 carbon atoms, such as a cyclopropyl group, a cyclopentyl group, a cyclohexyl group, a cyclopropylmethyl group, a 4-methylcyclohexyl group, a cyclohexylmethyl group, a norbornyl group, and an adamantyl groups; alkenyl groups having 2 to 20 carbon atoms, such as a vinyl group, a 1-propenyl group, a 2-propenyl group, a butenyl group, and a hexenyl group; unsaturated cyclic hydrocarbyl groups having 3 to 20 carbon atoms, such as a cyclohexenyl group; aryl groups having 6 to 20 carbon atoms, such as a phenyl group and a naphthyl group; aralkyl groups having 7 to 20 carbon atoms, such as a benzyl group, a 1-phenylethyl group, and a 2-phenylethyl group; and groups obtained by combining these groups. Furthermore, part or all of the hydrogen atoms of the hydrocarbyl group may be substituted with a group having a heteroatom, such as an oxygen atom, a sulfur atom, a nitrogen atom, or a halogen atom, and part of the —CH₂— of the hydrocarbyl group may be substituted with a group having a heteroatom, such as an oxygen atom, a sulfur atom, or a nitrogen atom. As a result, the hydrocarbyl group may have a hydroxy group, a fluorine atom, a chlorine atom, a bromine atom, an iodine atom, a cyano group, a carbonyl group, an ether bond, an ester bond, a sulfonate ester bond, a carbonate bond, a lactone ring, a sultone ring, a carboxylic anhydride (—C(═O)—O—C(═O)—), a haloalkyl group, etc.

In the general formula (a), L^{A} and L^{B} represent each independently a single bond, an ether bond, an ester bond, an amide bond, a sulfonate ester bond, a sulfonamide bond, a carbonate bond, or a carbamate bond. Among these, L^{A} is preferably a single bond, an ether bond, or an ester bond, and L^{B} is preferably an ester bond or a sulfonate ester bond, and more preferably *—C(═O)—O—** (“*” represents an appointment point to W¹ or W², and “**” represents an appointment point to benzenesulfonic acid). However, L^{B} is more preferably located at the p-position relative to the sulfonate anion. If the sterically bulky W²-L^{A}-W¹-L^{B}- or W²-L^{B}- is located at the o-position or m-position relative to the sulfonate anion, effective acid generation from the sulfonate anion moiety is inhibited.

In the general formula (a), W¹ represents a hydrocarbylene group having 1 to 40 carbon atoms and not involving resonance structures, and may have a heteroatom. W¹ is preferably a divalent group having 3 to 40 carbon atoms that has a lactone ring or a sultone ring structure, and more preferably a divalent group having 6 to 12 carbon atoms that contains a lactone ring or a sultone ring structure. By locating the lactone ring or sultone ring in a position close to the sulfonic acid group during acid generation after exposure, acid diffusion can be further suppressed.

Specific examples of the group represented by W¹ include, but are not limited to, the following.

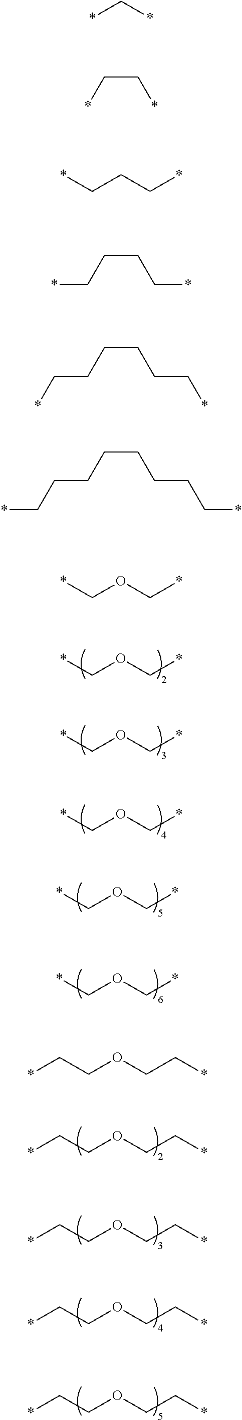

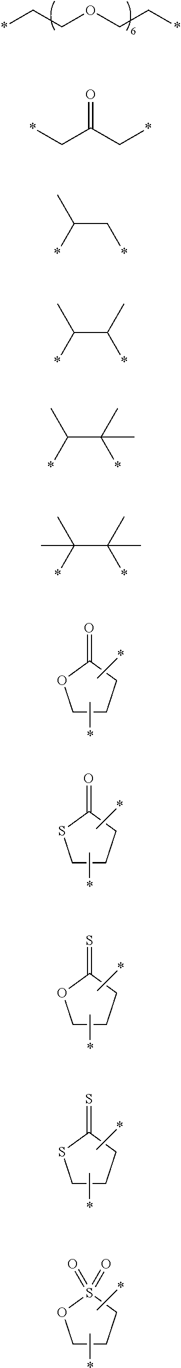

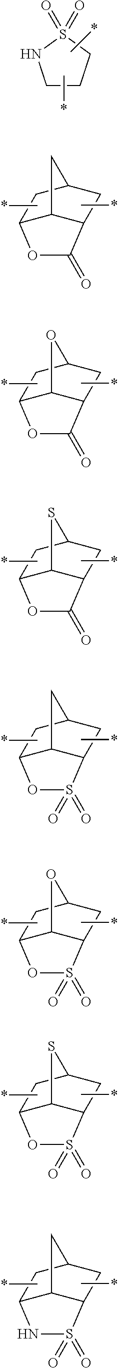

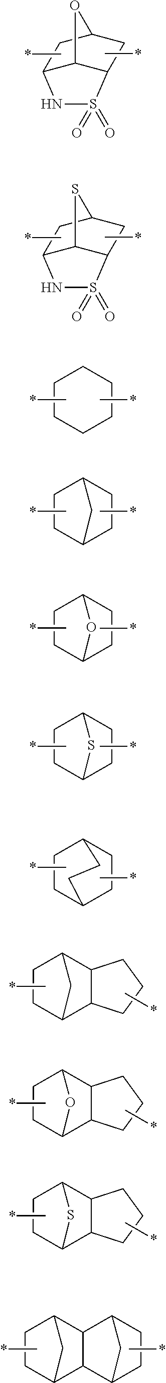

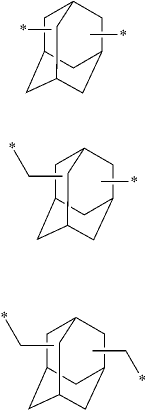

“*” represents an attachment point.

Among these, W¹ is preferably X^{L}-0 to X^{L}-22, X^{L}-29 to X^{L}-34, and X^{L}-47 to X^{L}-49.

In the general formula (a), W² represents a hydrocarbylene group having 1 to 40 carbon atoms and not involving resonance structures, and may have a heteroatom. W² is preferably a monovalent group having 7 to 14 carbon atoms containing a polycyclic alicyclic structure having 7 to 14 carbon atoms.

Specific examples of the group represented by W² include, but are not limited to, the following.

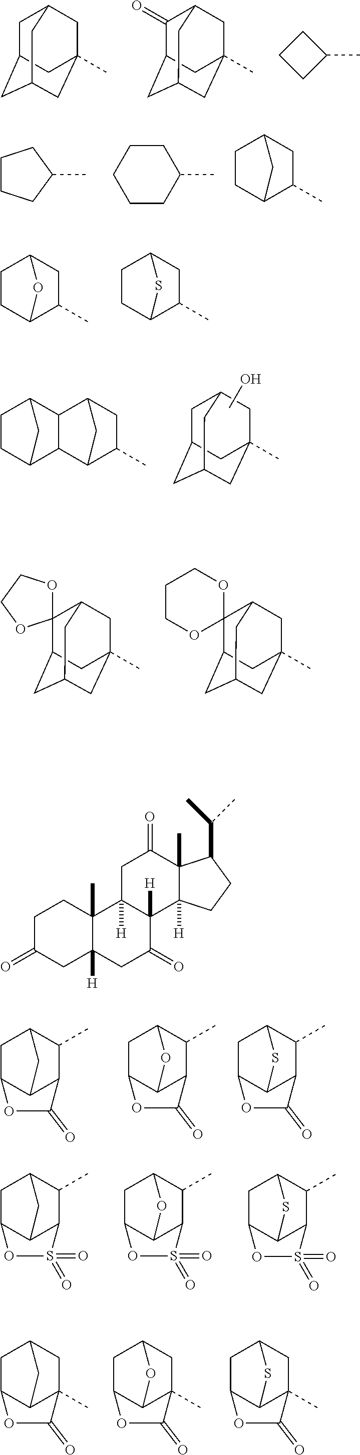

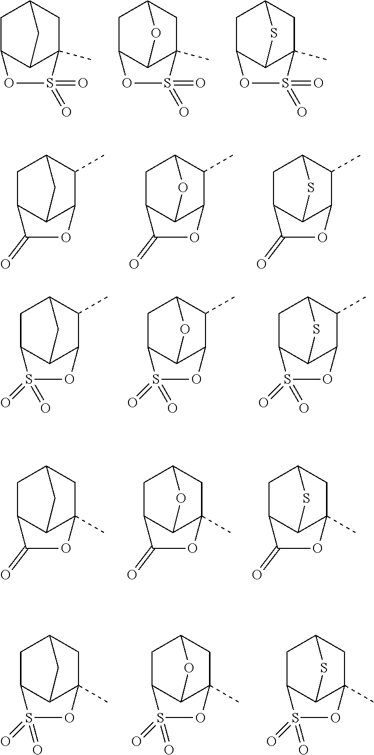

The broken line represents an attachment point.

At least one of W¹ and W² preferably have an alicyclic structure having 3 to 40 carbon atoms and optionally having a hetero atom. Both of W¹ and W² more preferably have an alicyclic structure having 3 to 40 carbon atoms and optionally having a hetero atom. Such a photoacid generator can effectively suppress acid diffusion.

The onium salt represented by the above general formula (a) is preferably one represented by the following general formula (a1). In such onium salts, the benzene ring to which the sulfonate anion is bonded is substituted with an iodine atom, and its electron-withdrawing properties increase the acidity, which is expected to result in high sensitivity. Furthermore, a structure in which an iodine atom is bonded to an aromatic ring is expected to be a material with lower environmental load than fluoroalkanesulfonic acid. “m2a” represents an integer of 1 to 4, but is preferably 2 or greater. Such an onium salt has high acid strength.

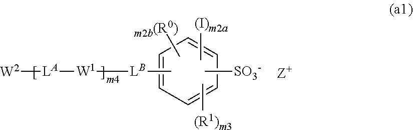

In the formula, “m3”, “m4”, L^{A}, L^{B}, R⁰, R¹, W¹, W², and Z⁺ represent the same as defined above, “m2a” represents an integer of 1 to 4, and “m2b” represents an integer of 0 to 3.

The onium salt represented by the general formula (a) is preferably represented by the following general formula (a2).

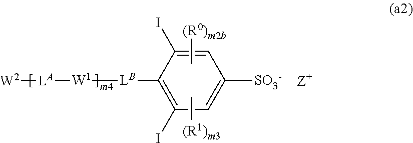

In the formula, “m4”, L^{A}, L^{B}, R⁰, R¹, W¹, W², and Z⁺ represent the same as defined above, and “m2b” and “m3” represent an integer of 0 to 2.

Specific examples of the anion of the onium salt represented by the above general formula (a) are shown below, but are not limited thereto.

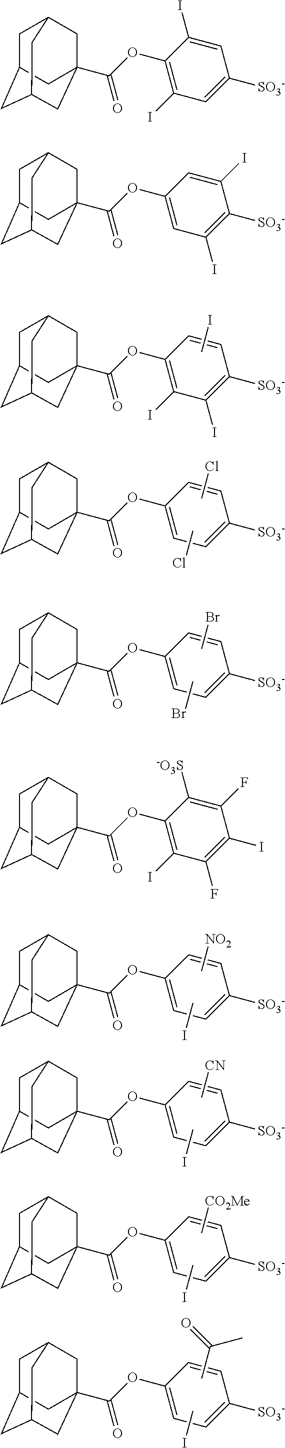

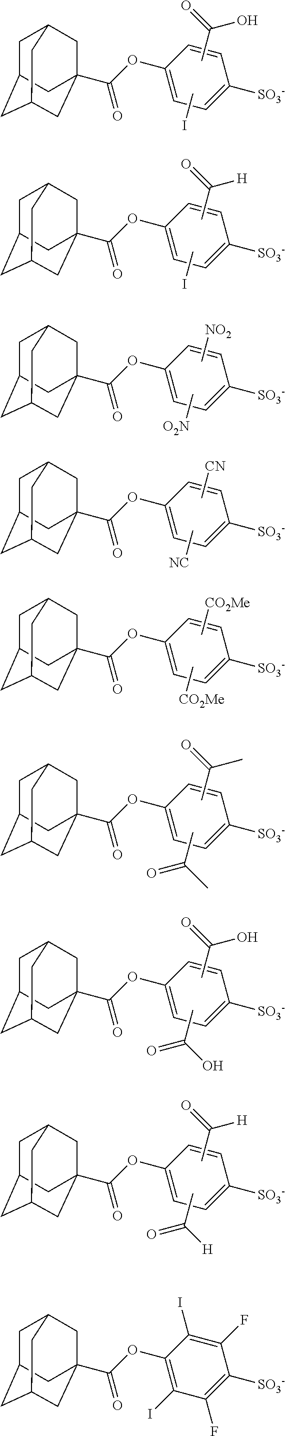

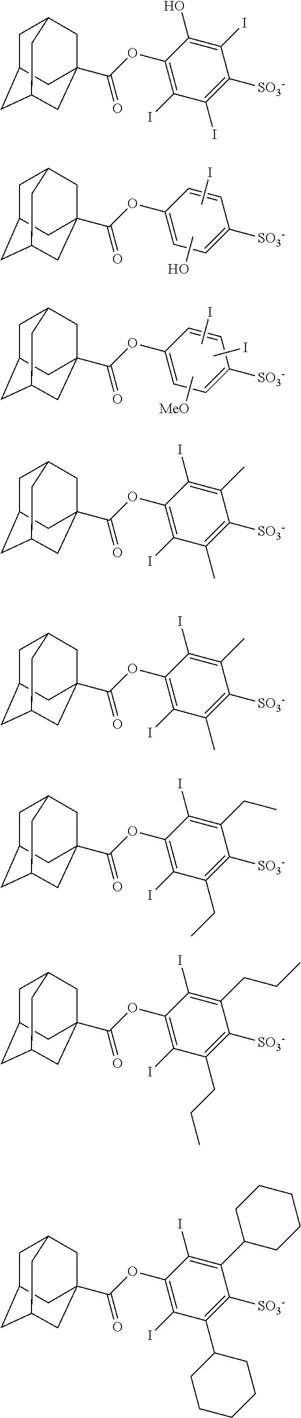

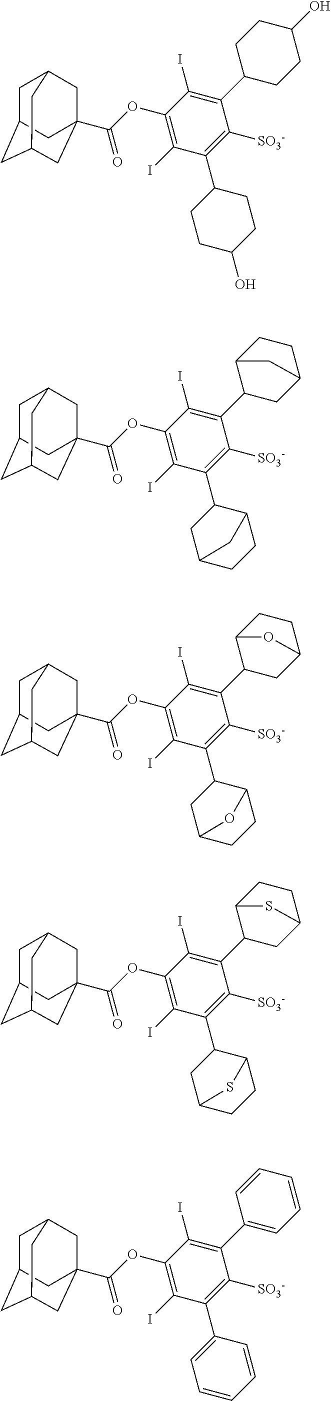

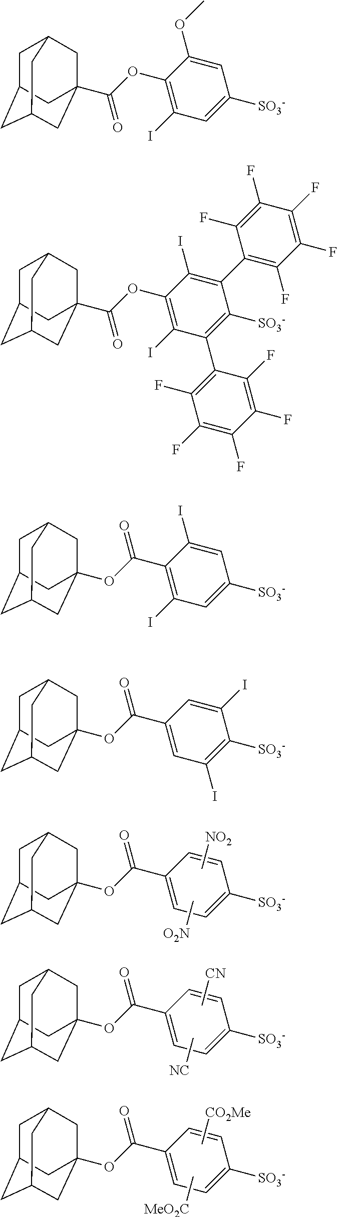

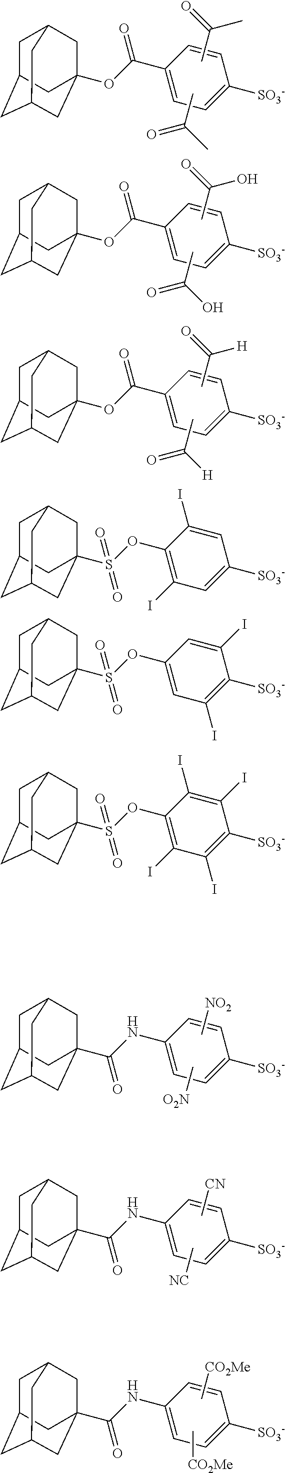

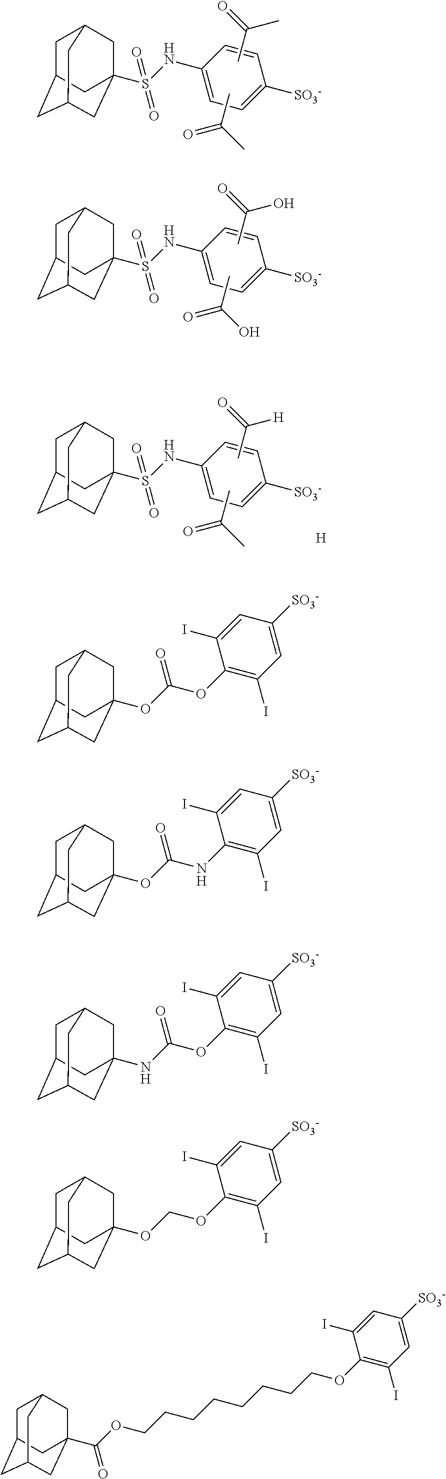

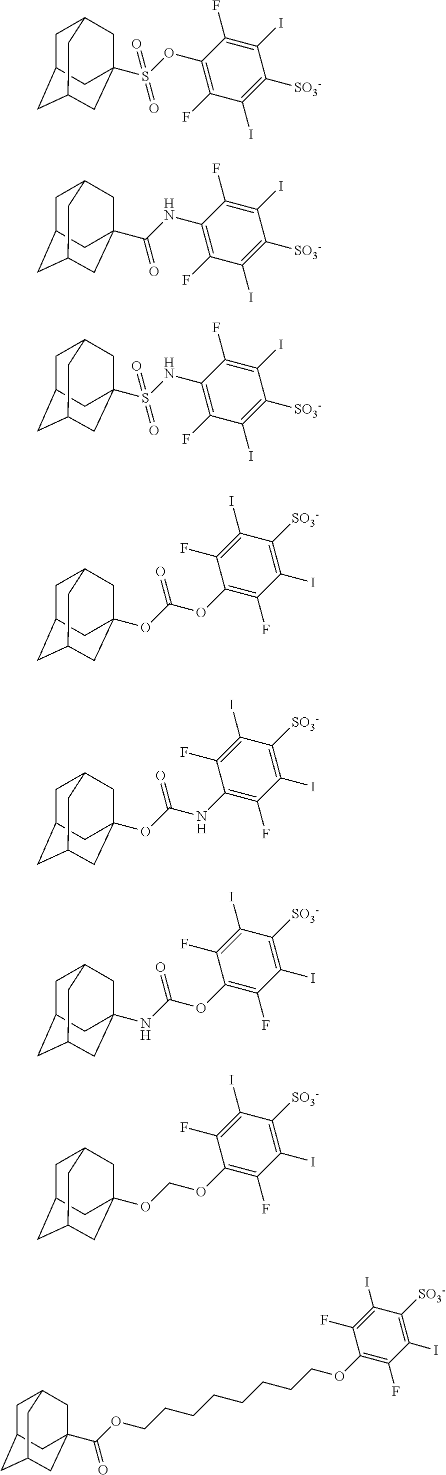

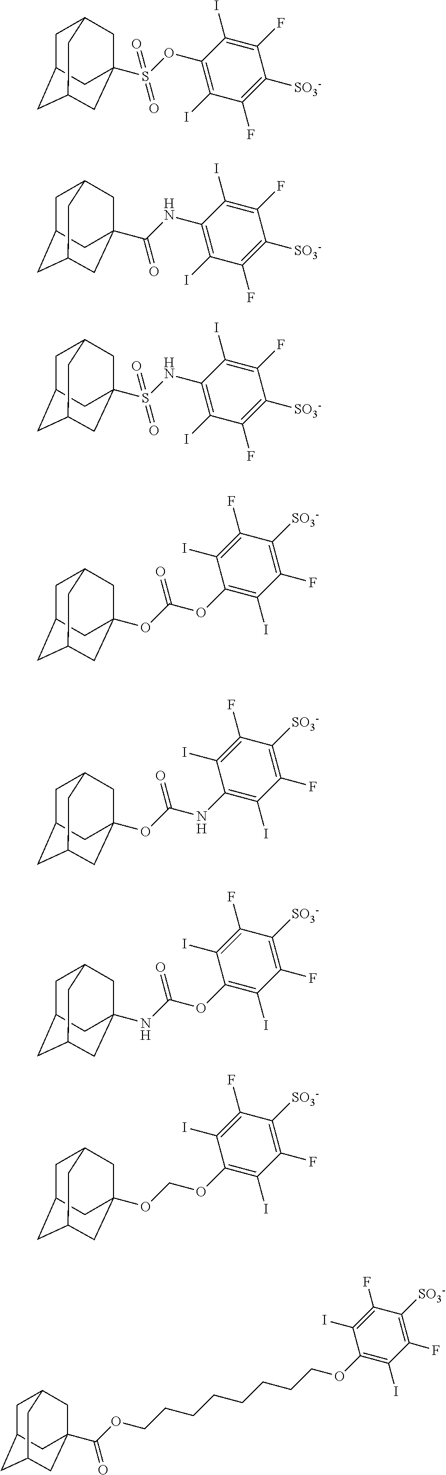

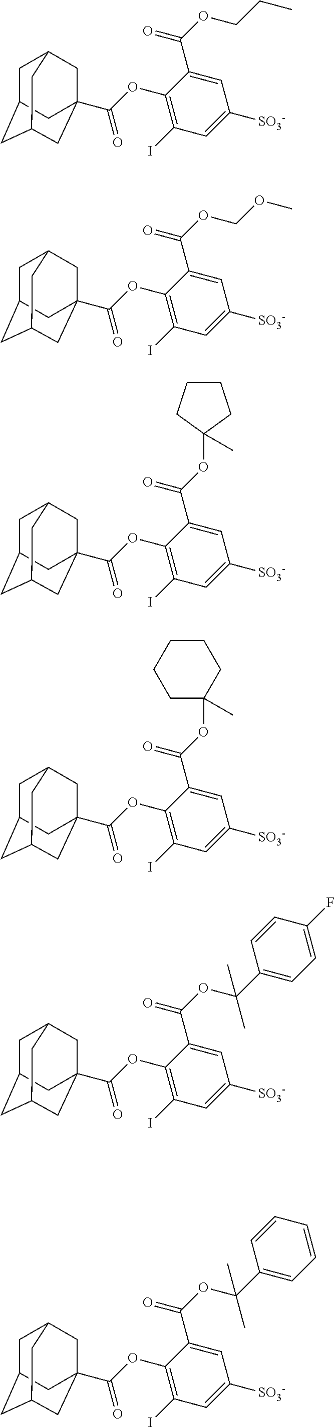

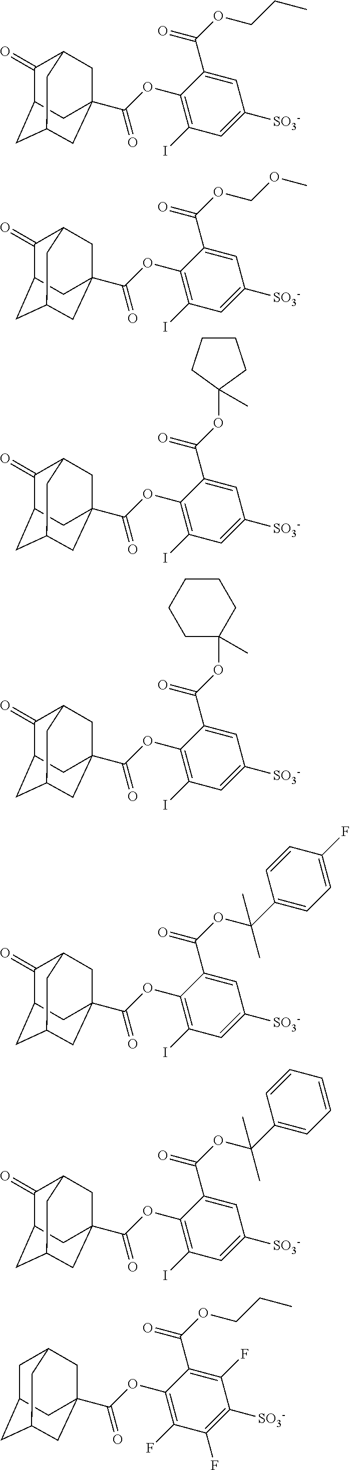

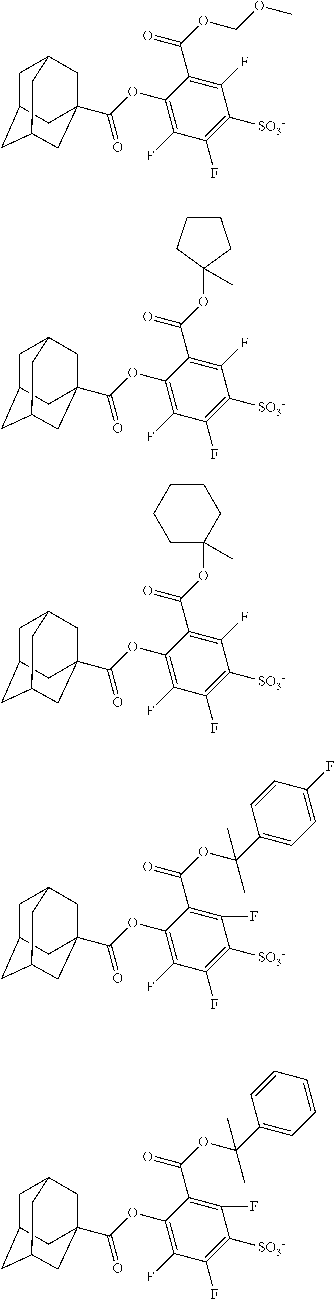

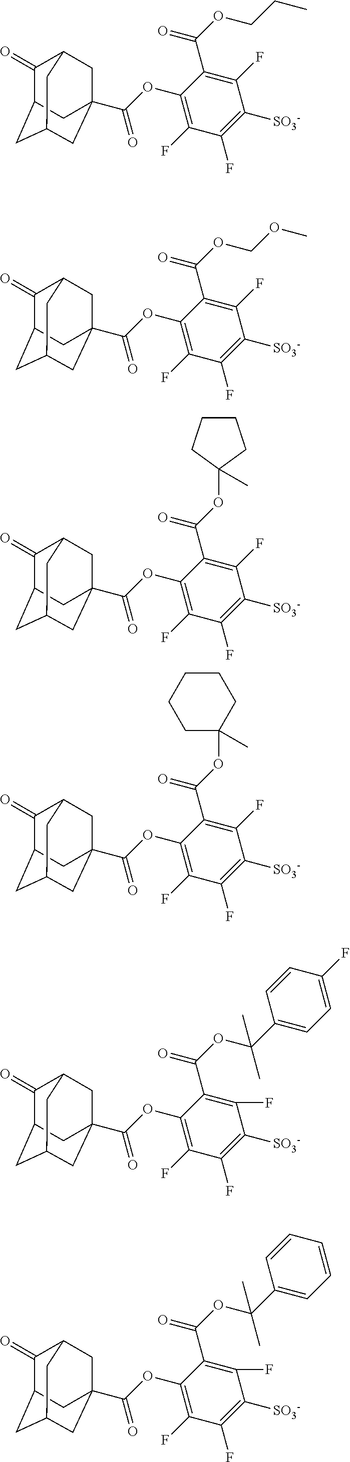

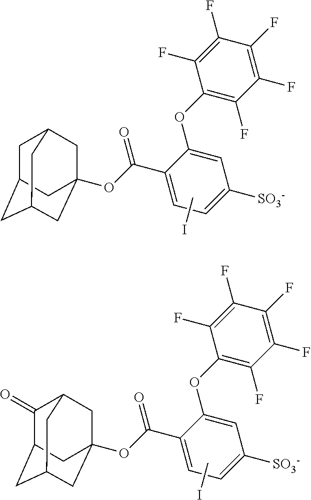

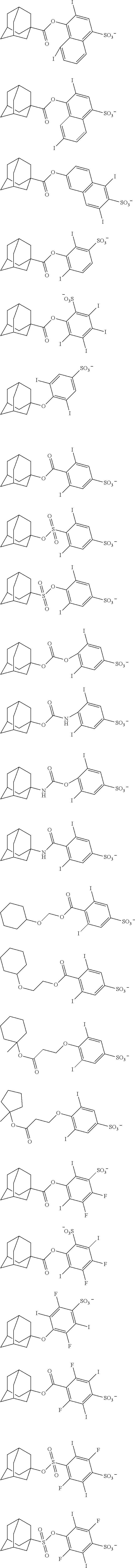

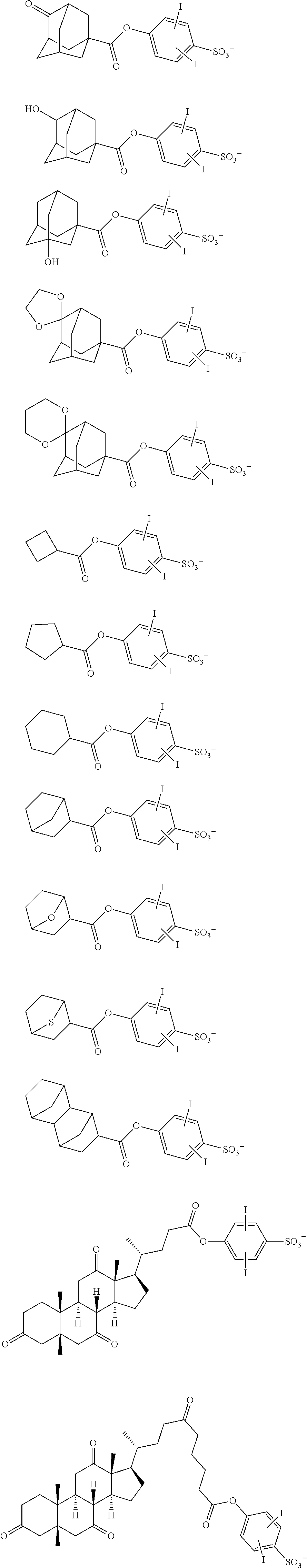

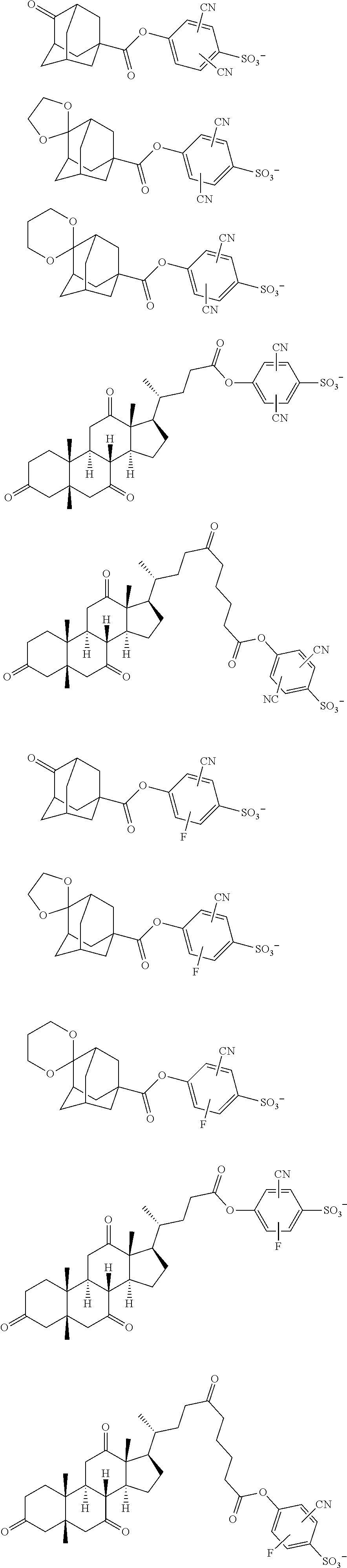

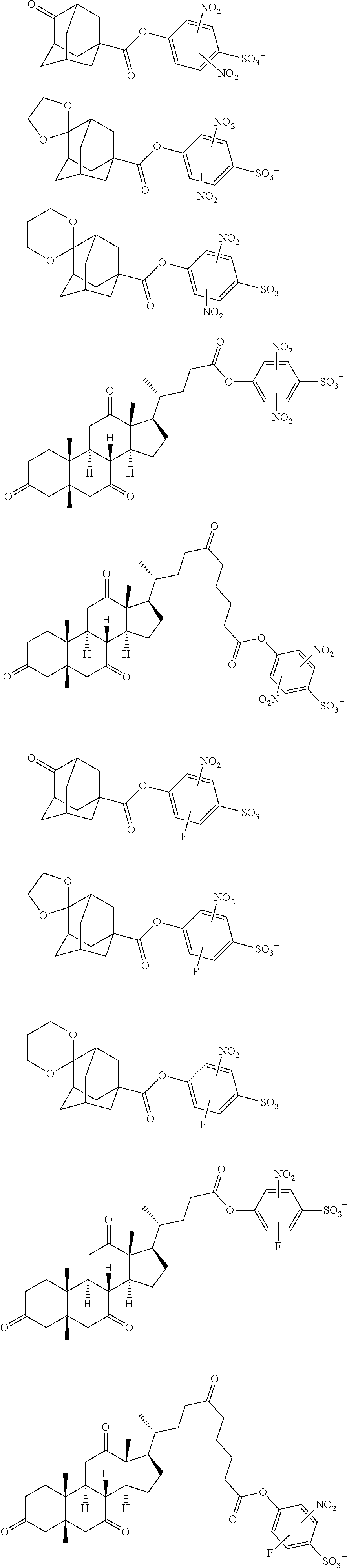

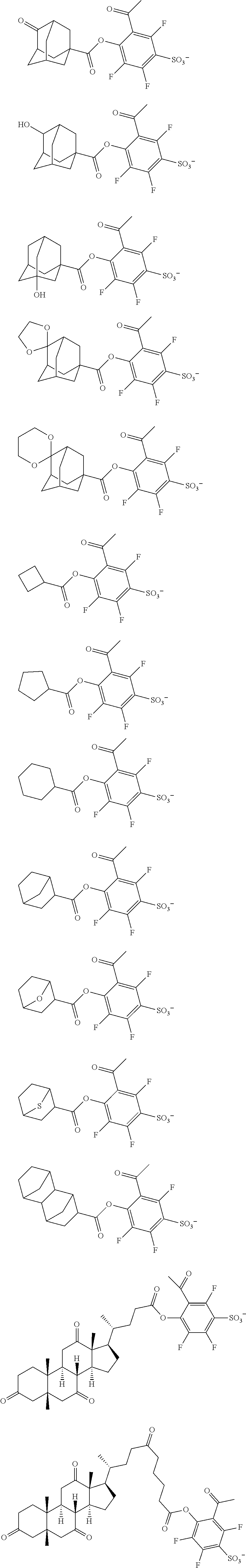

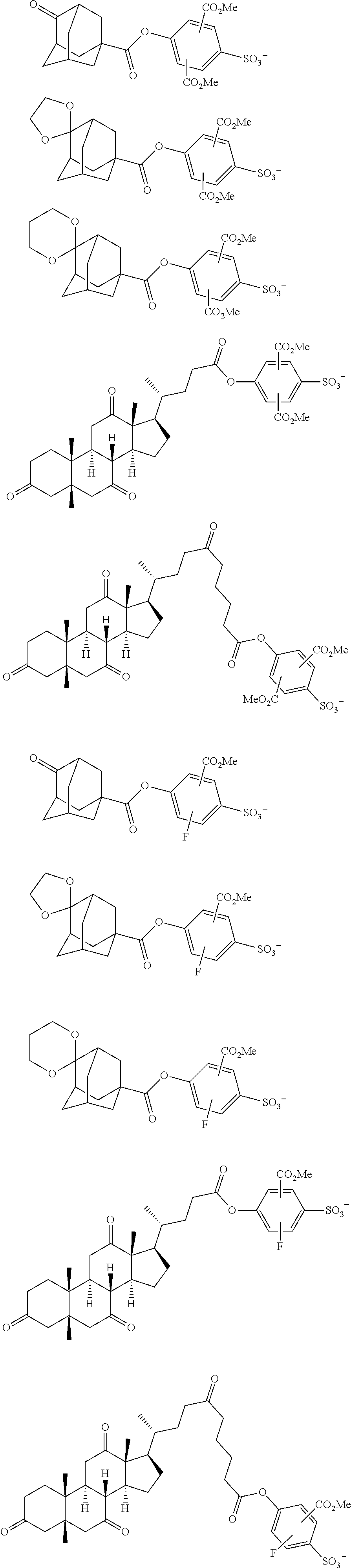

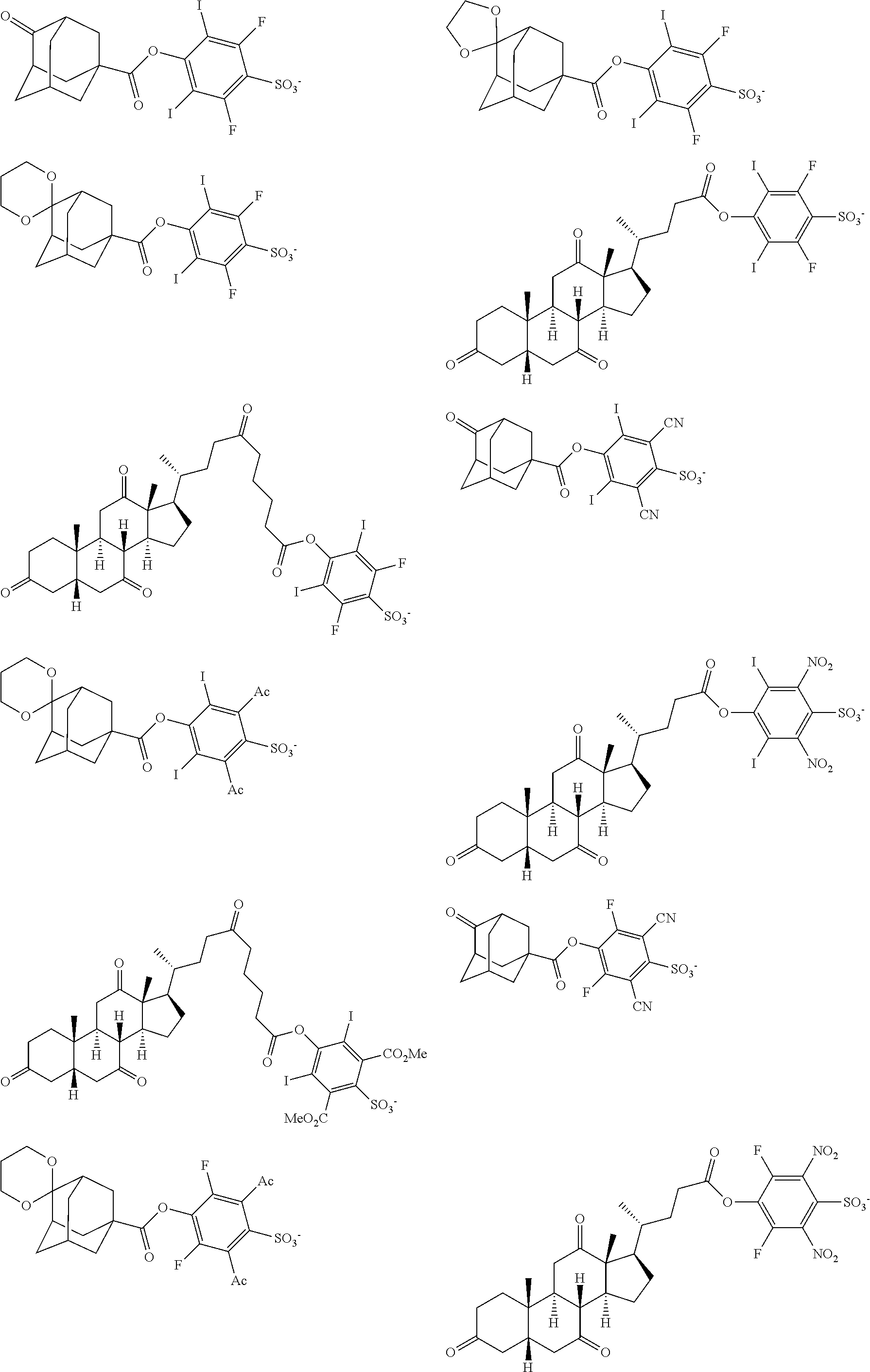

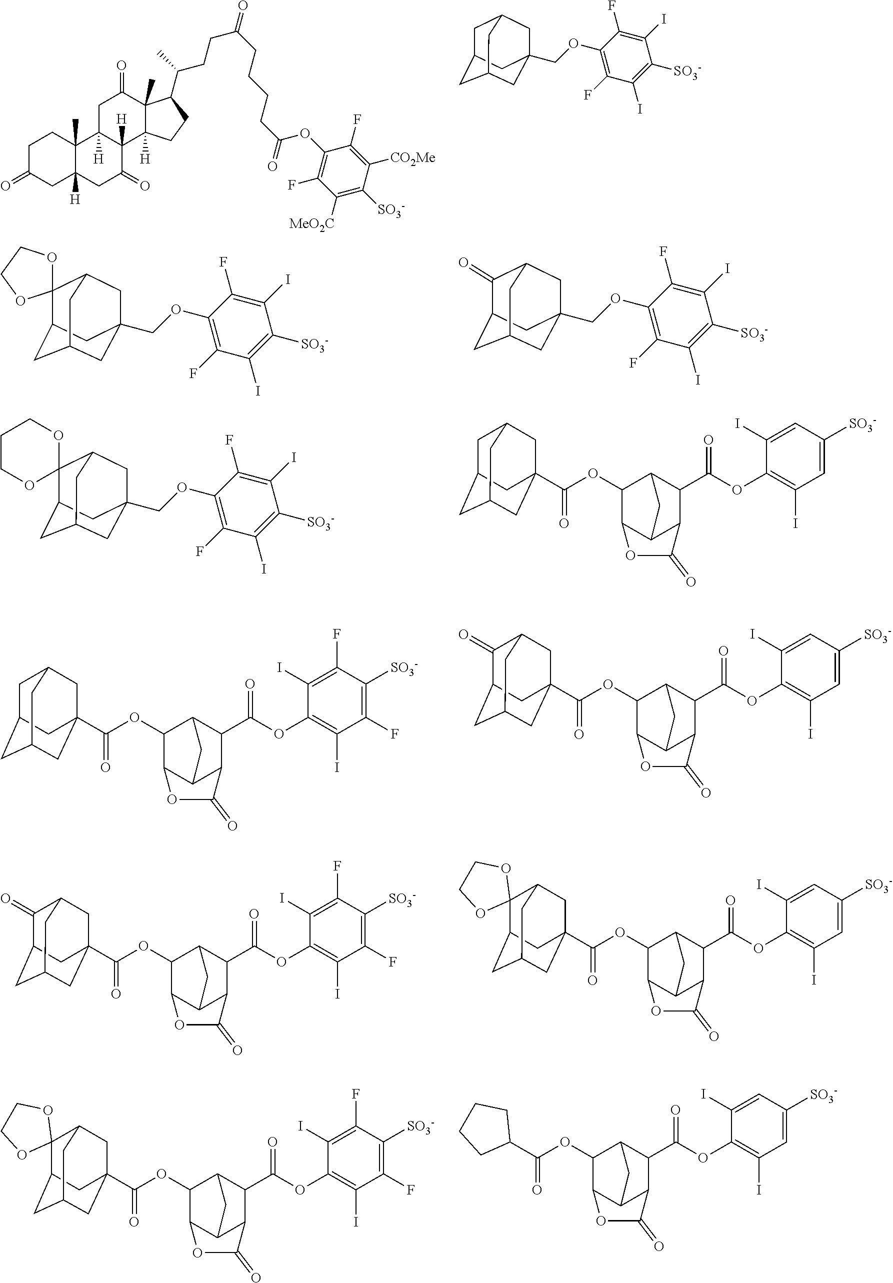

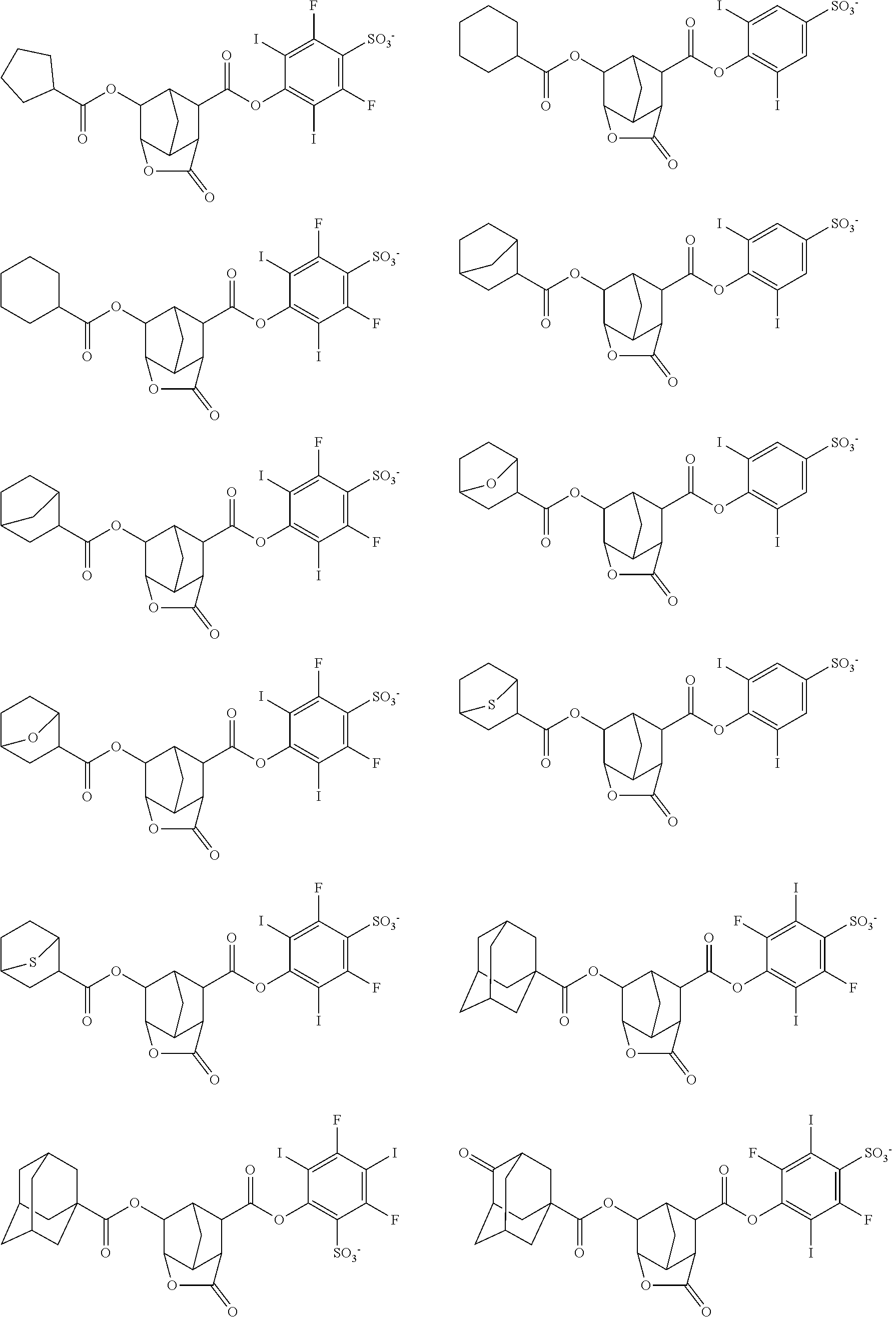

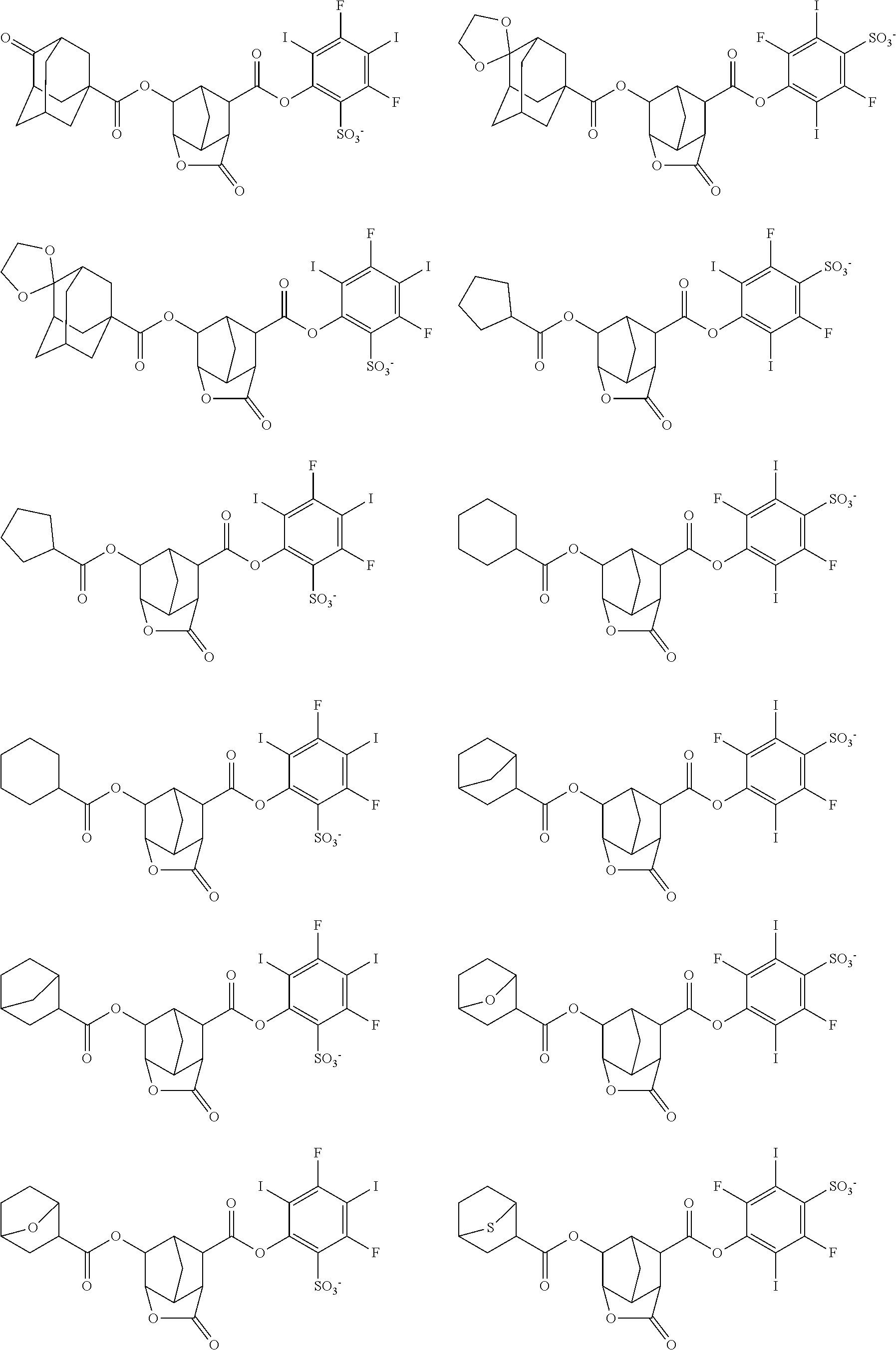

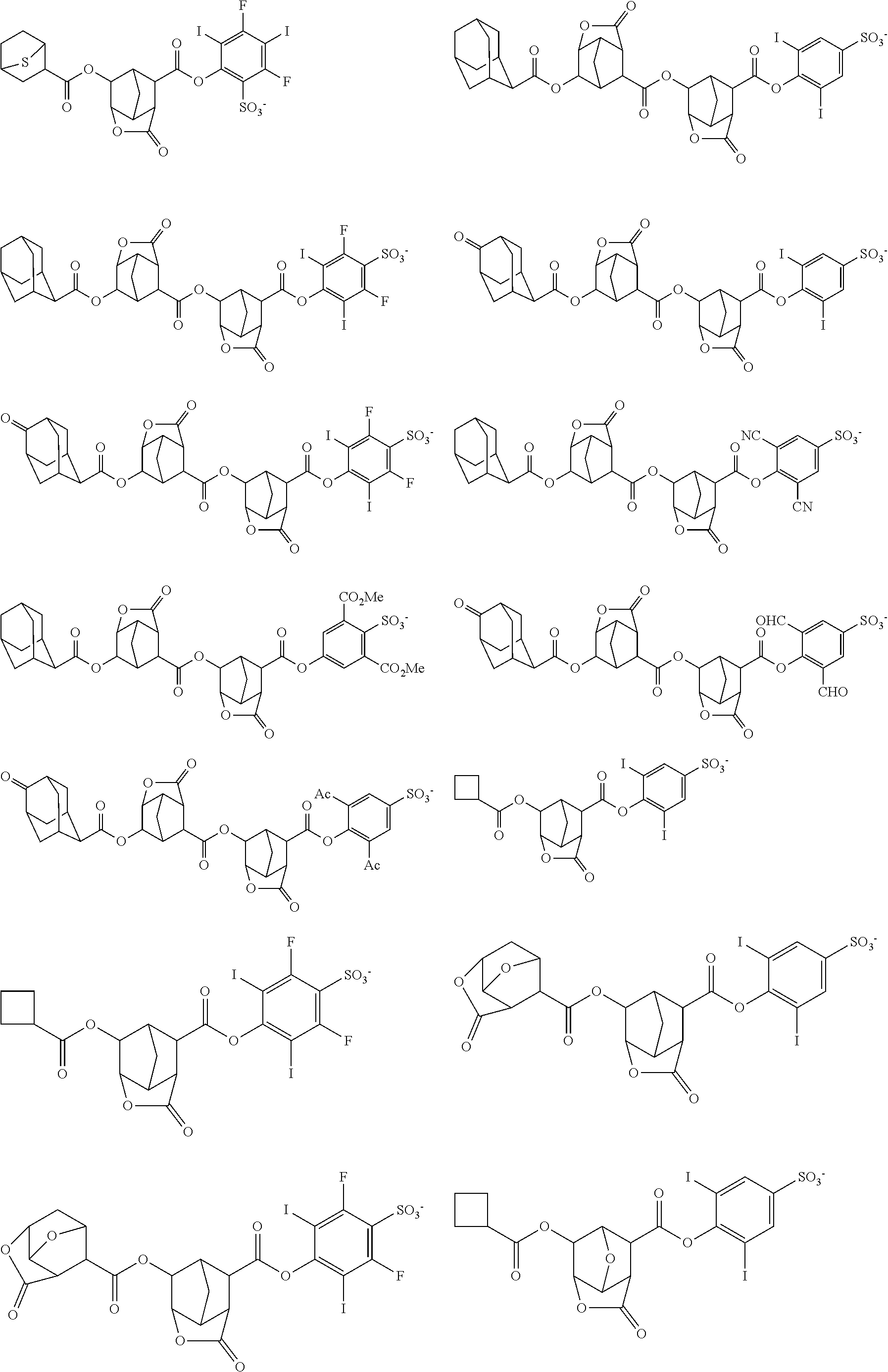

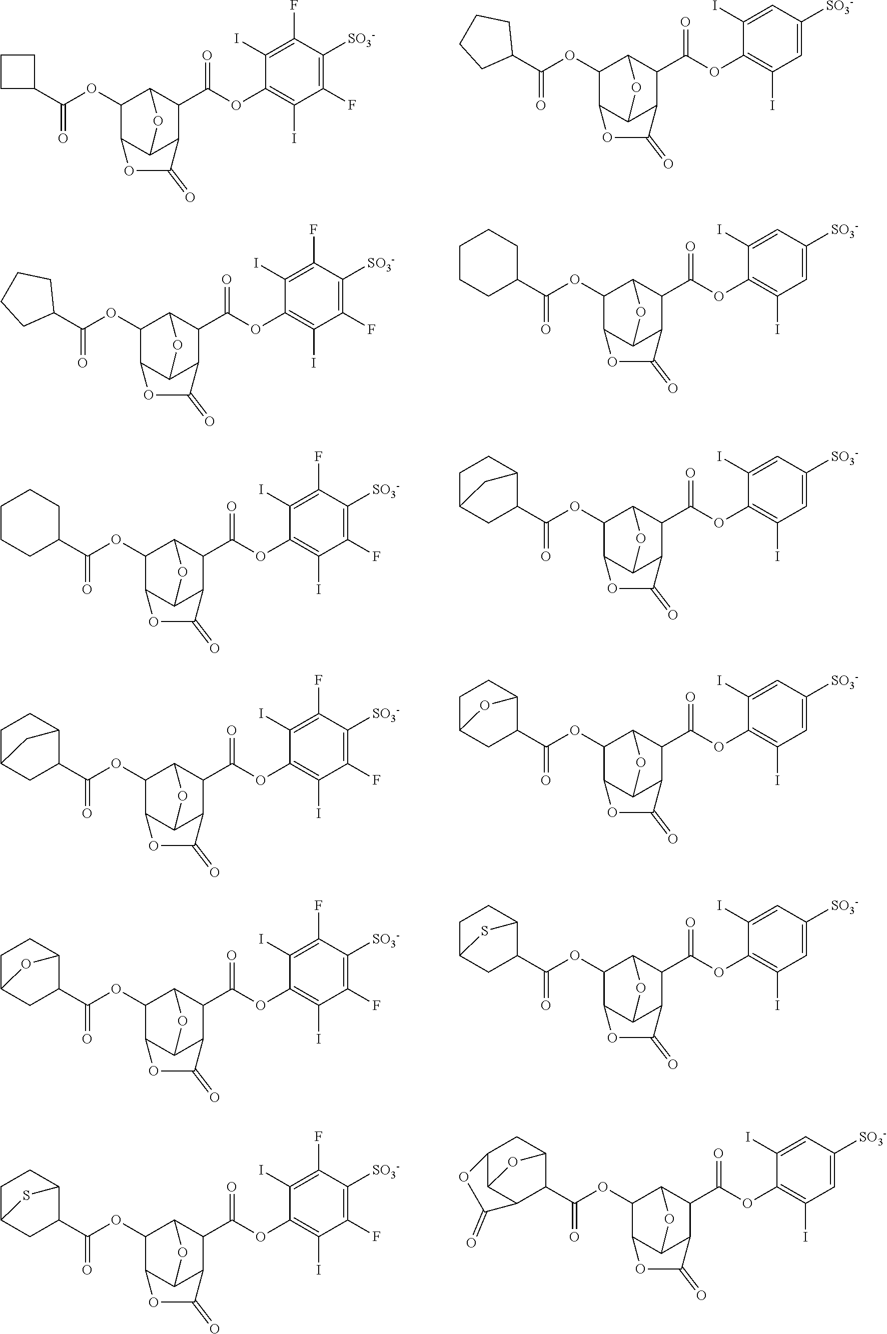

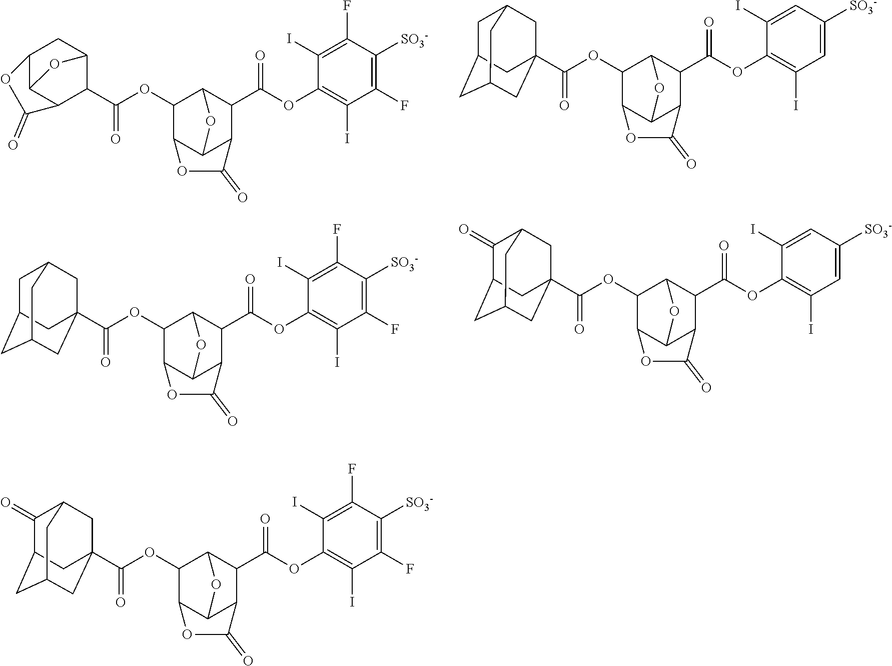

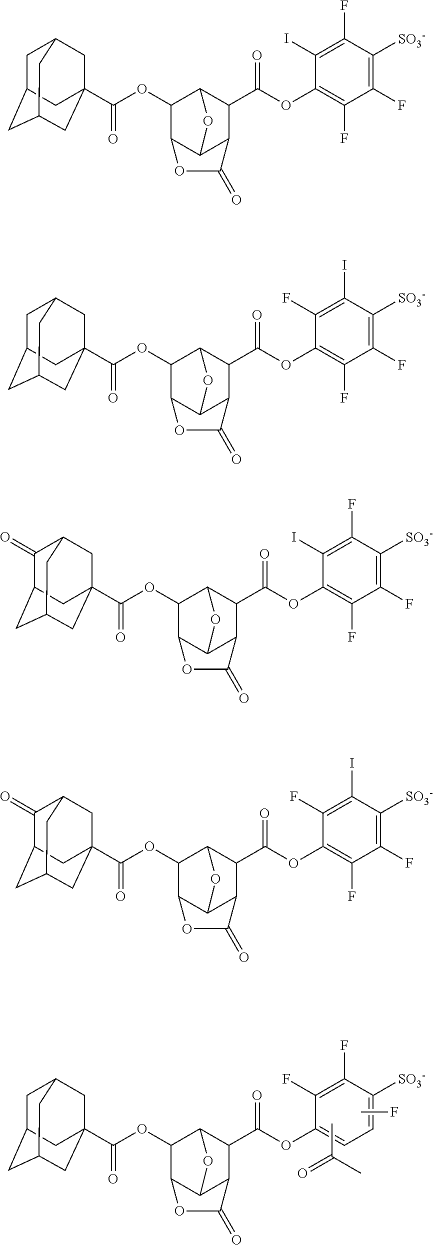

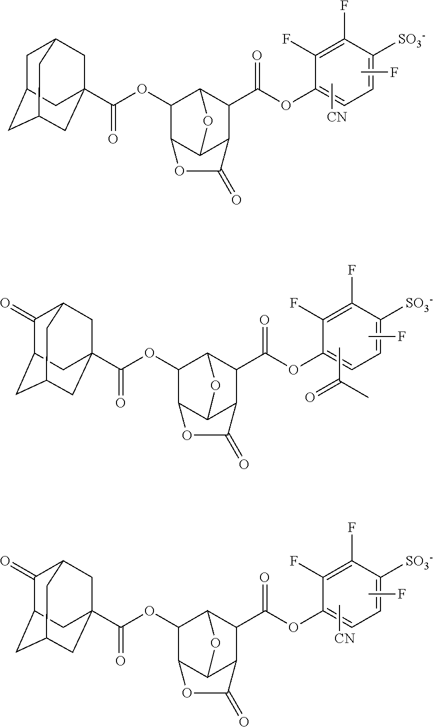

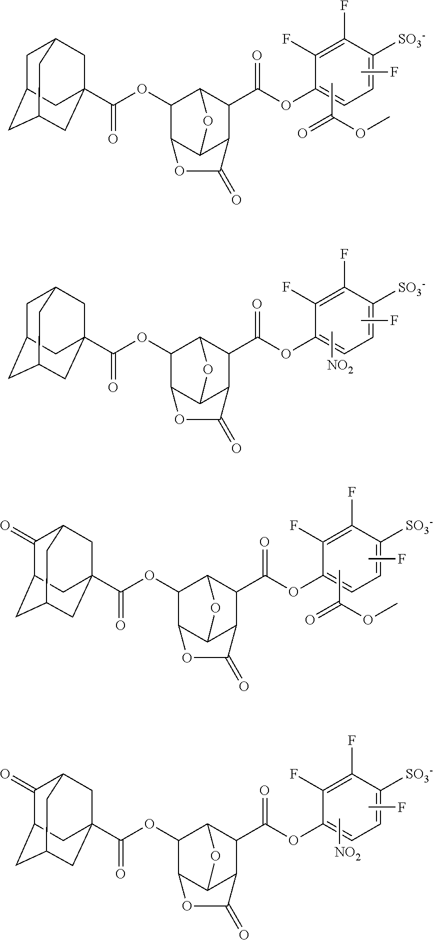

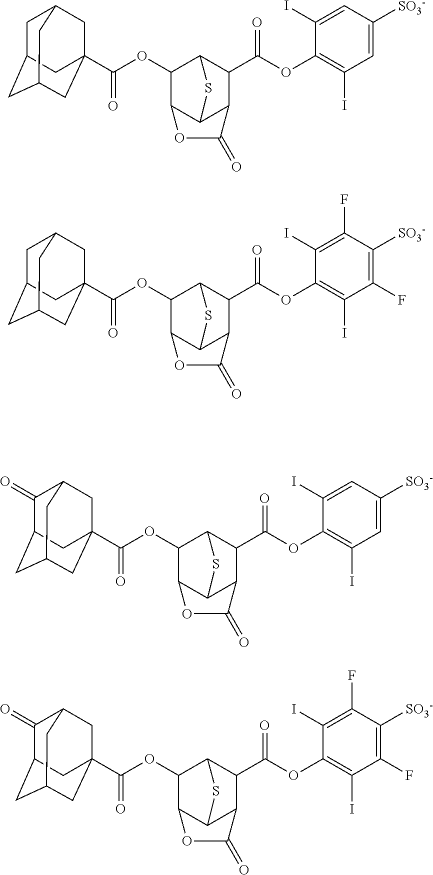

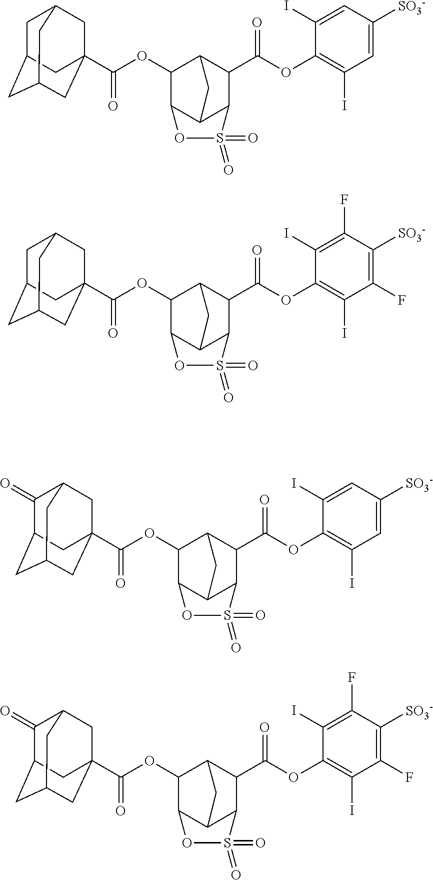

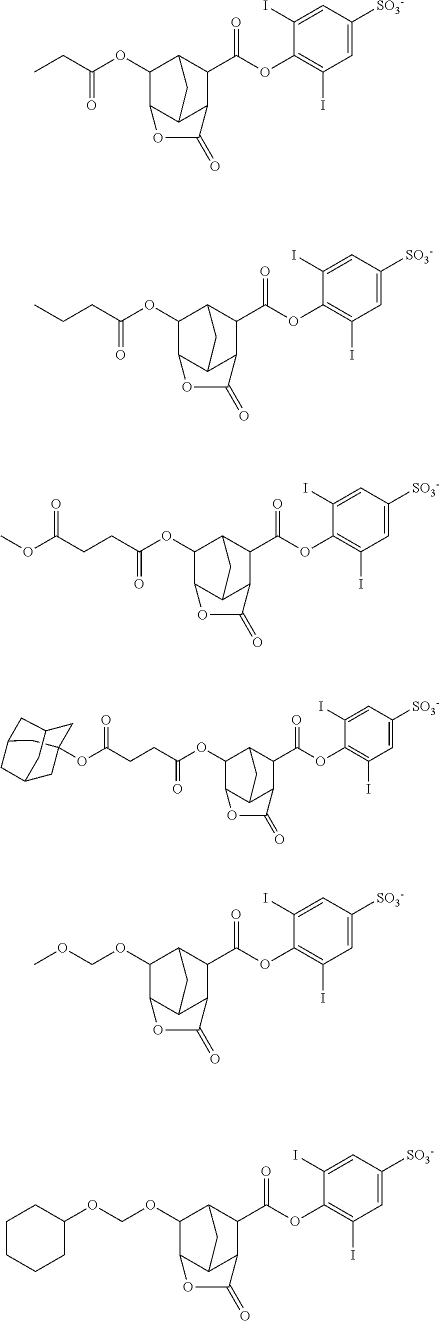

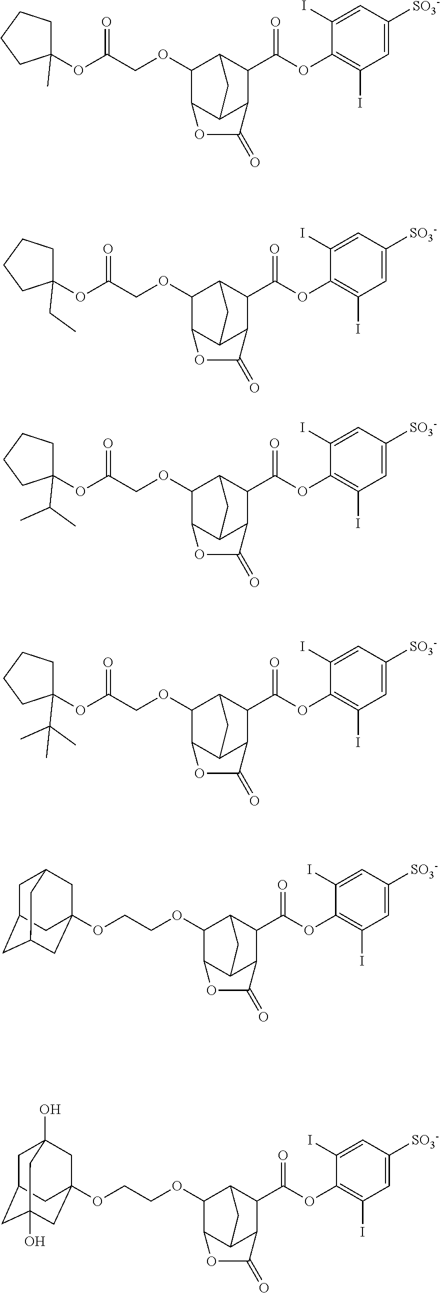

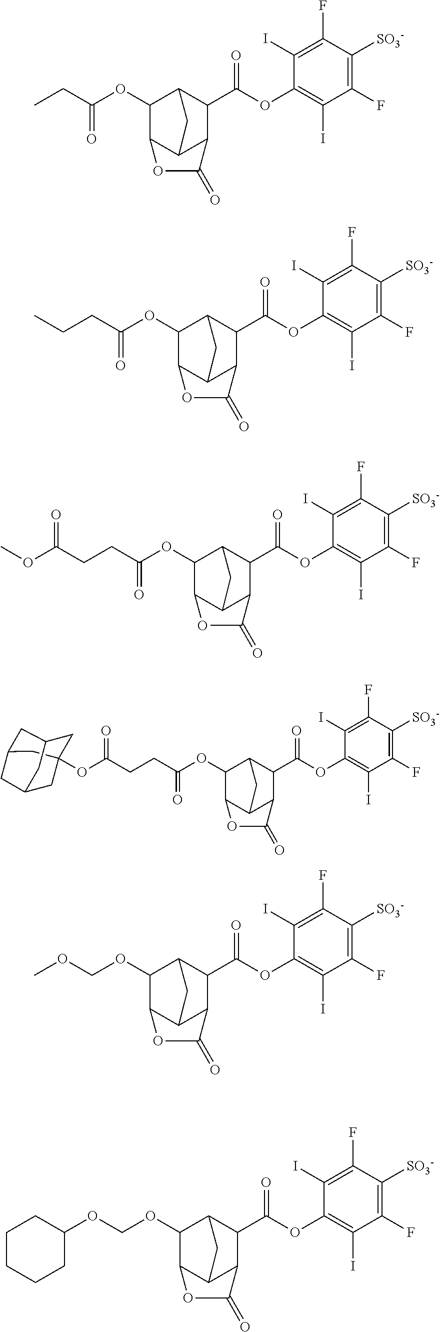

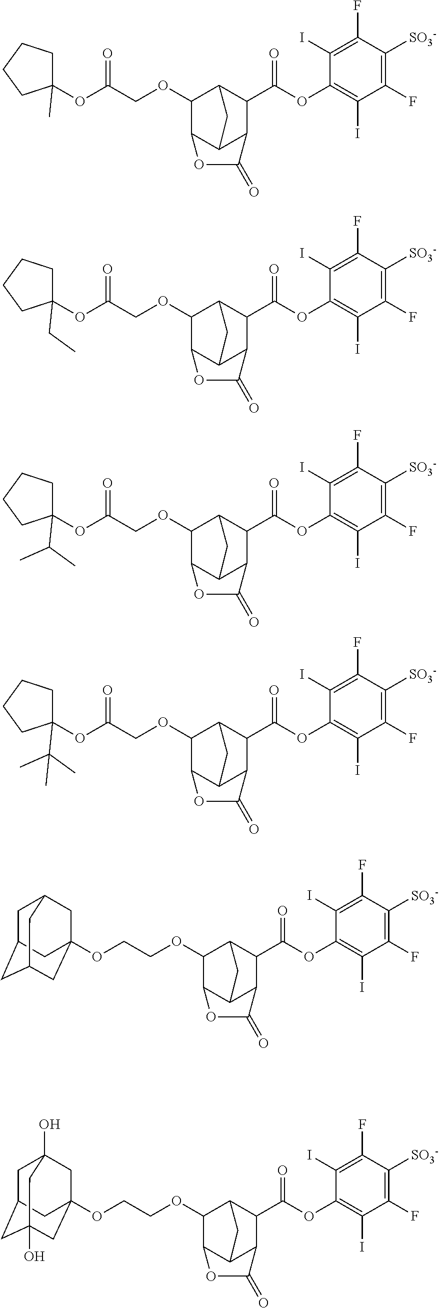

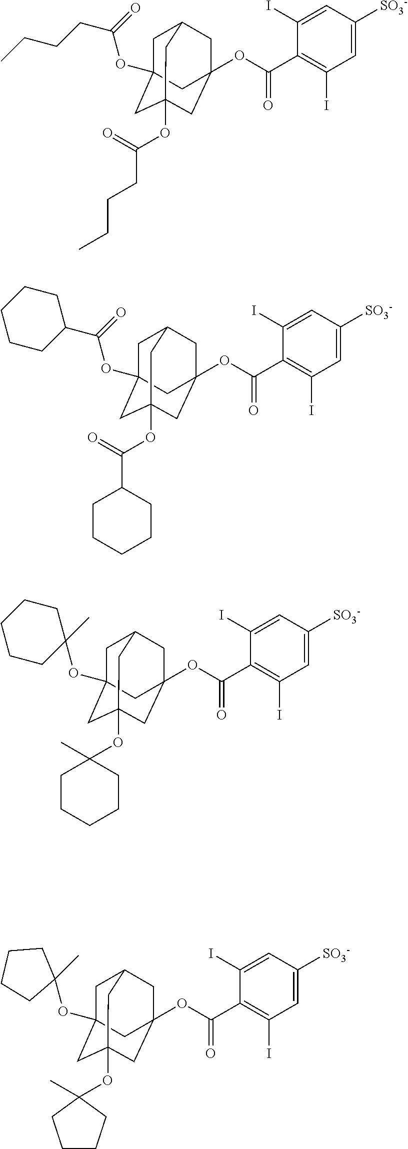

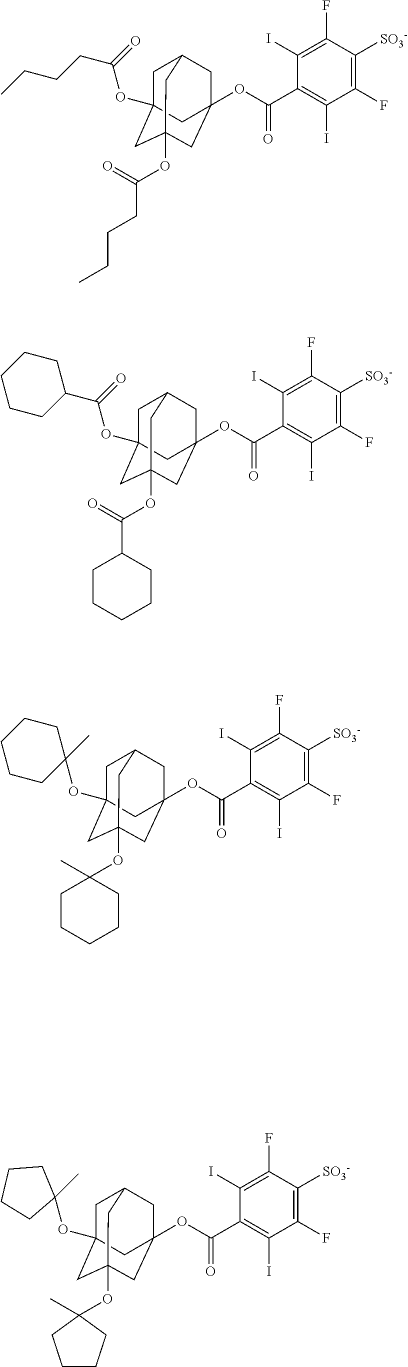

In the general formula (a), Z⁺ represents a sulfonium cation represented by the following general formula (Z-1) or an iodonium cation represented by the following general formula (Z-2).

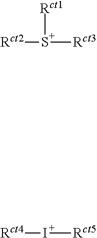

In the formula,
R^{ct1} to R^{ct5} each independently represent a halogen atom or a hydrocarbyl group having 1 to 30 carbon atoms and optionally having a heteroatom, and R^{ct1} and R^{ct2} may be bonded to each other to form a ring together with the sulfur atom to which they are bonded.

In the general formulae (Z-1) and (Z-2), R^{ct1} to R^{ct5} each independently represent a halogen atom or a hydrocarbyl group having 1 to 30 carbon atoms and optionally having a heteroatom.

Specific examples of the halogen atom represented by R^{ct1} to R^{ct5} include a fluorine atom, a chlorine atom, a bromine atom, and an iodine atom.

The hydrocarbyl group represented by R^{ct1} to R^{ct5} may be saturated or unsaturated, and may be linear, branched, or cyclic. Specific examples thereof include: alkyl groups having 1 to 30 carbon atoms, such as a methyl group, an ethyl group, an n-propyl group, an isopropyl group, an n-butyl group, an isobutyl group, a sec-butyl group, and a tert-butyl group; saturated cyclic hydrocarbyl groups having 3 to 30 carbon atoms, such as a cyclopropyl group, a cyclopentyl group, a cyclohexyl group, a cyclopropylmethyl group, a 4-methylcyclohexyl group, a cyclohexylmethyl group, a norbornyl group, and an adamantyl group; alkenyl groups having 2 to 30 carbon atoms, such as a vinyl group, an allyl group, a propenyl group, a butenyl group, and a hexenyl group; unsaturated cyclic hydrocarbyl groups having 3 to 30 carbon atoms, such as a cyclohexenyl group; aryl groups having 6 to 30 carbon atoms, such as a phenyl group, a naphthyl group, and a thienyl group; aralkyl groups having 7 to 30 carbon atoms, such as a benzyl group, a 1-phenylethyl group, and a 2-phenylethyl group; and groups obtained by combinations of these groups. Aryl groups are preferable. Furthermore, part or all of the hydrogen atoms of the hydrocarbyl group may be substituted with a group having a heteroatom, such as an oxygen atom, a sulfur atom, a nitrogen atom, or a halogen atom, and part of the —CH₂— of the hydrocarbyl group may be substituted with a group having a heteroatom, such as an oxygen atom, a sulfur atom, or a nitrogen atom. As a result, the hydrocarbyl group may have a hydroxy group, a fluorine atom, a chlorine atom, a bromine atom, an iodine atom, a cyano group, a nitro group, a carbonyl group, an ether bond, an ester bond, a sulfonic acid ester bond, a carbonate bond, a lactone ring, a sultone ring, a carboxylic anhydride (—C(═O)—O—C(═O)—), a haloalkyl group, etc.

Furthermore, R^{ct1} and R^{ct2} may be bonded to each other to form a ring together with the sulfur atom to which they are bonded. In this case, specific examples of the structure of the ring include those represented by the following formulae.

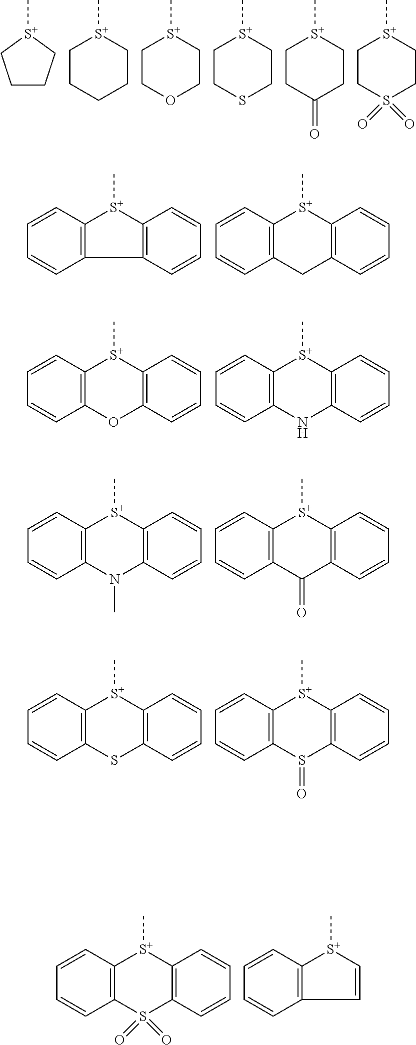

In the formulae, the broken line represents an attachment point to R^{ct3}.

Specific examples of the sulfonium cation represented by the general formula (Z-1) include those shown below, but are not limited thereto.

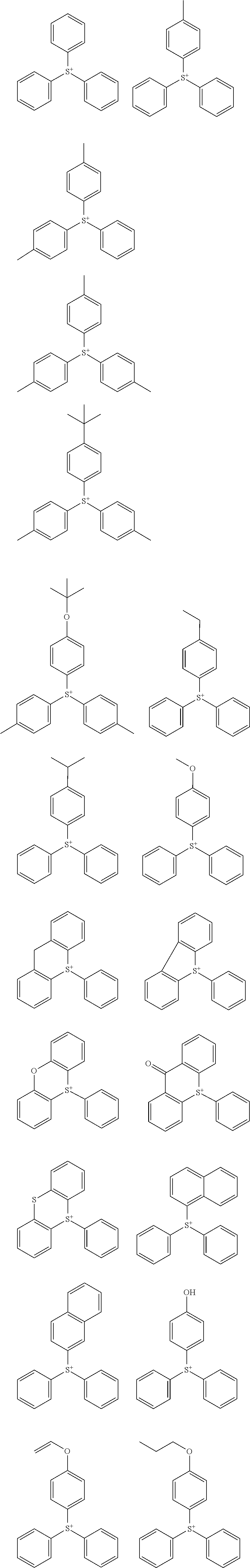

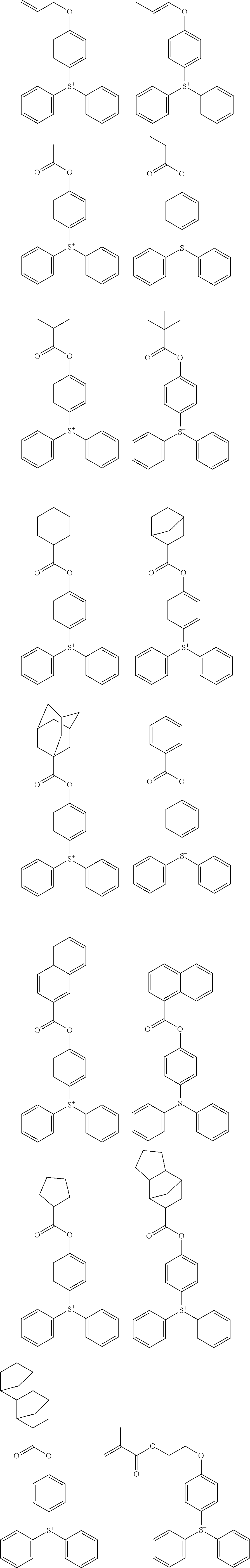

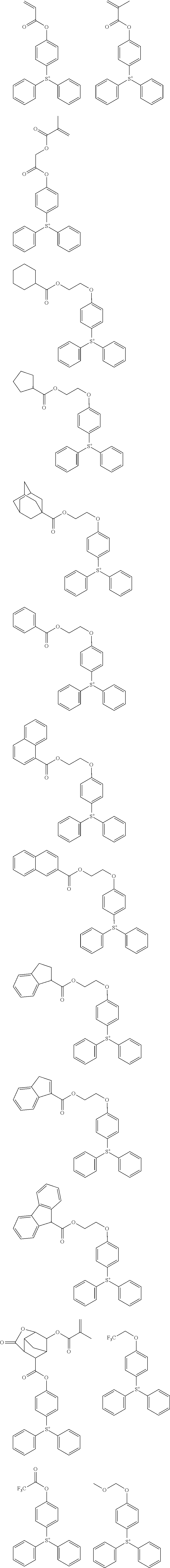

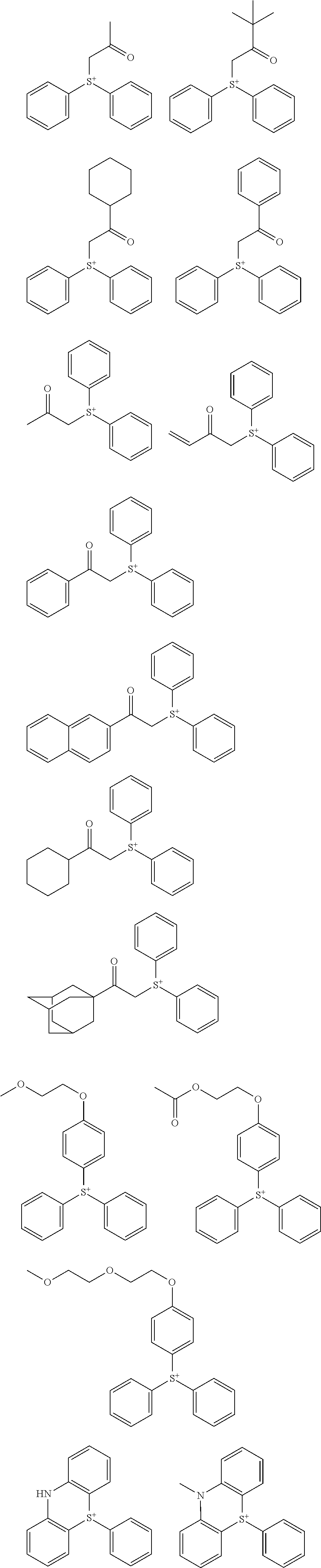

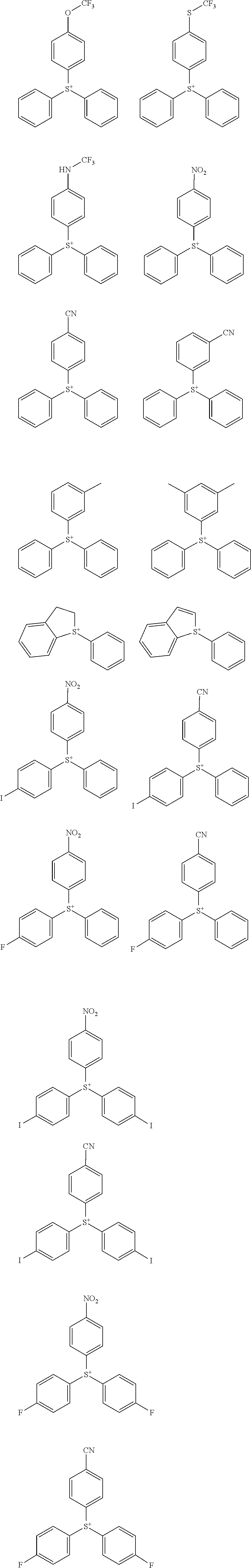

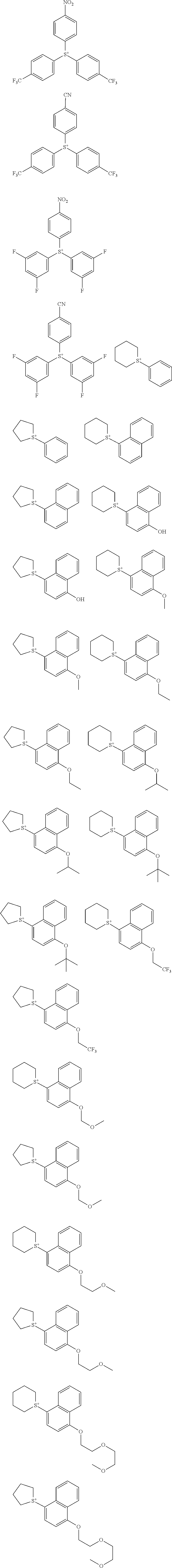

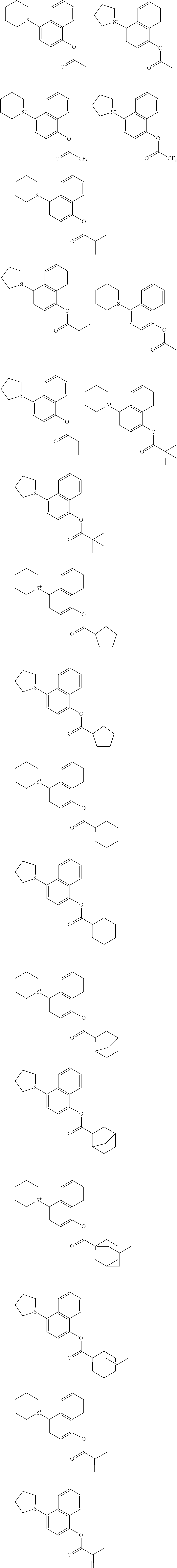

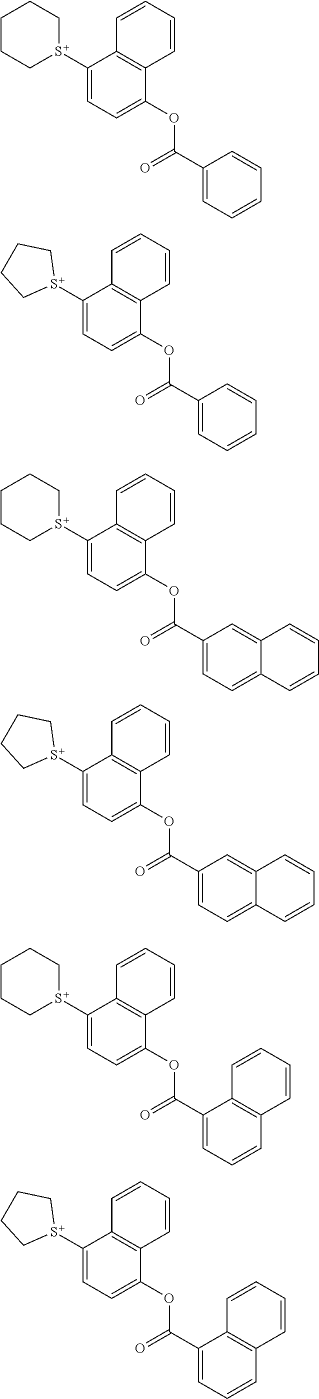

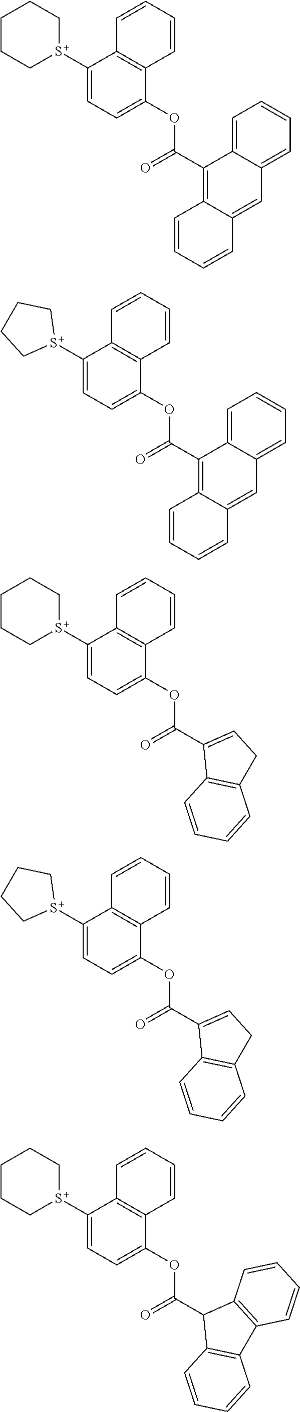

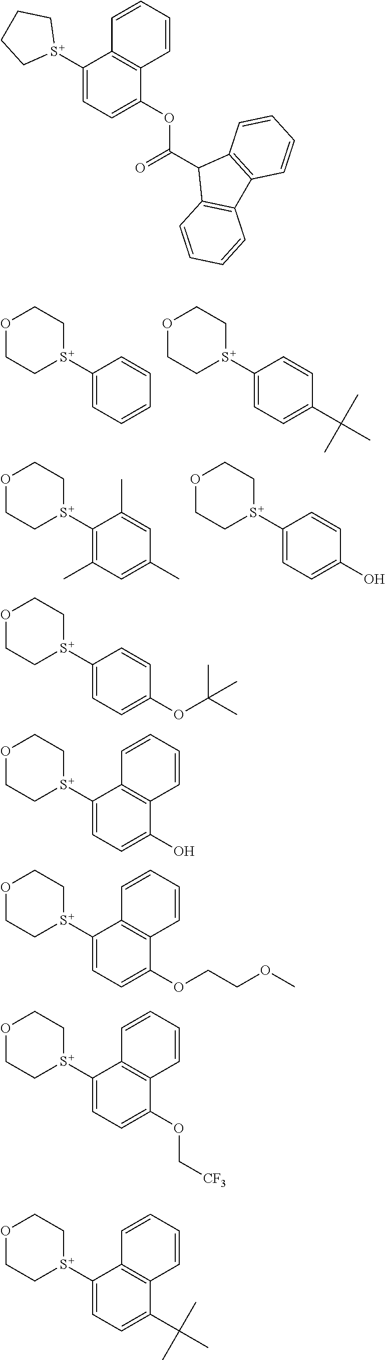

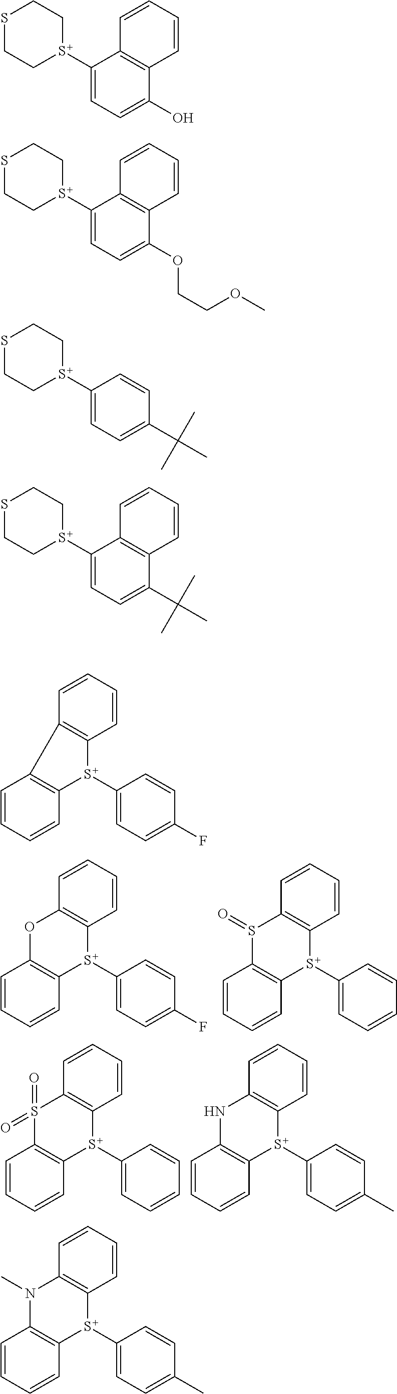

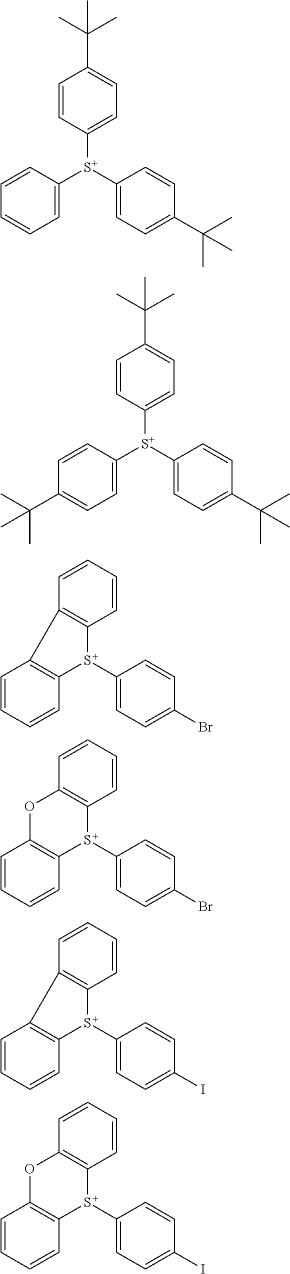

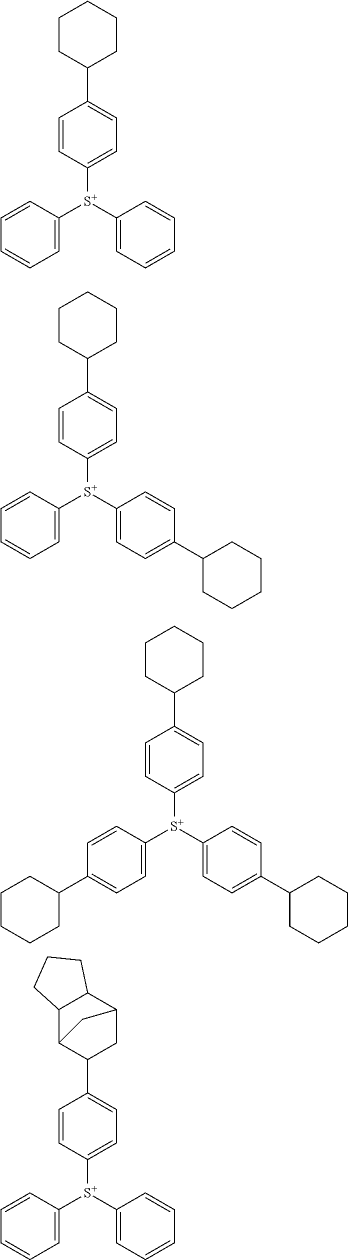

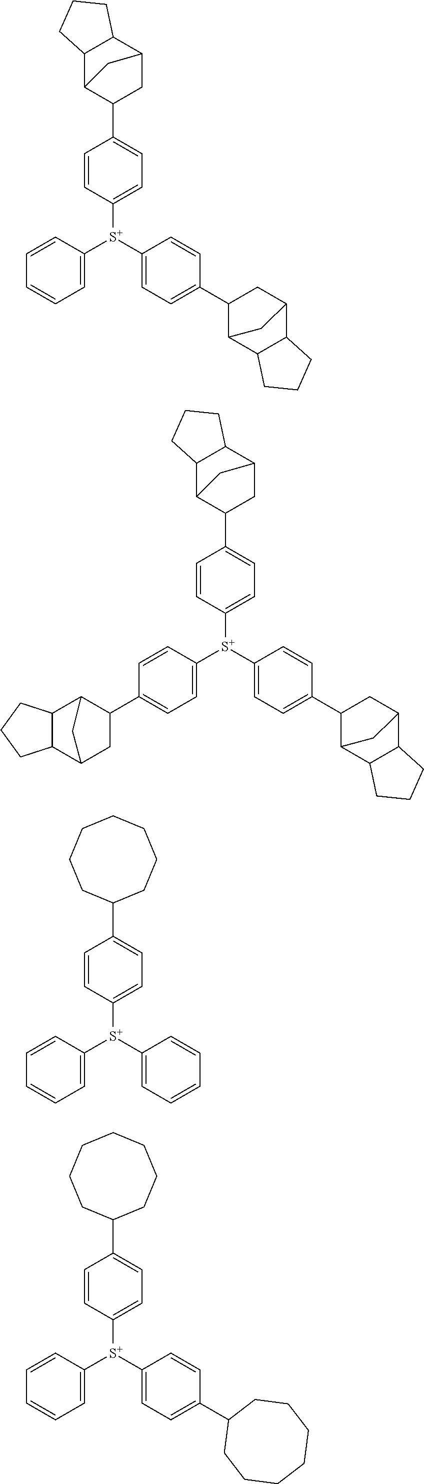

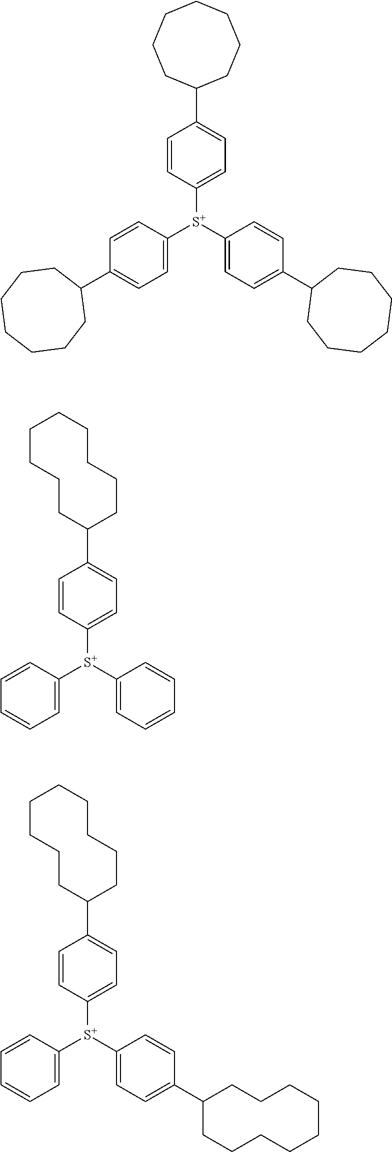

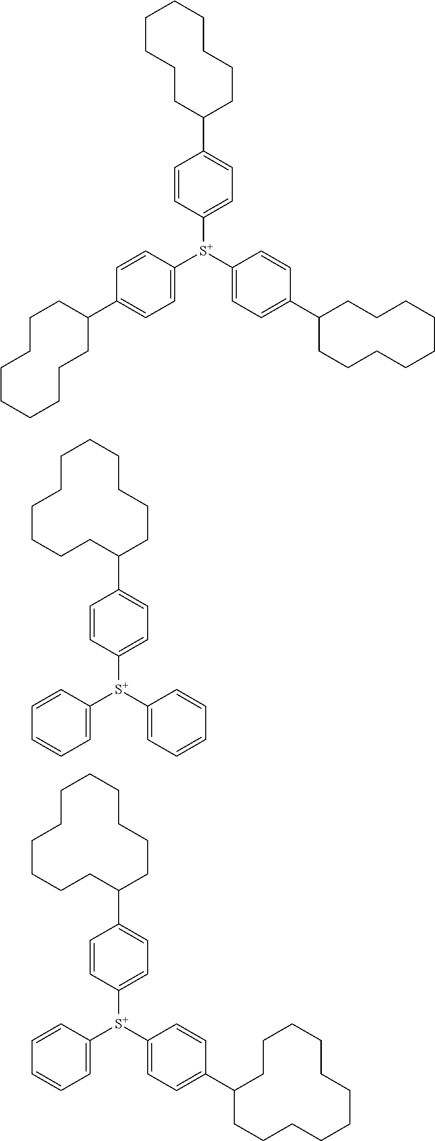

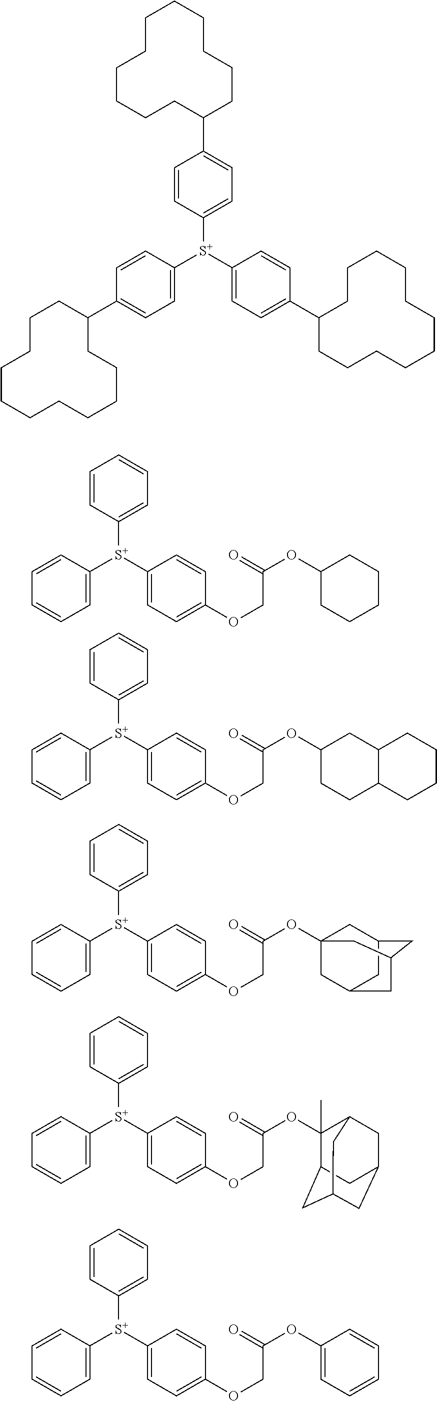

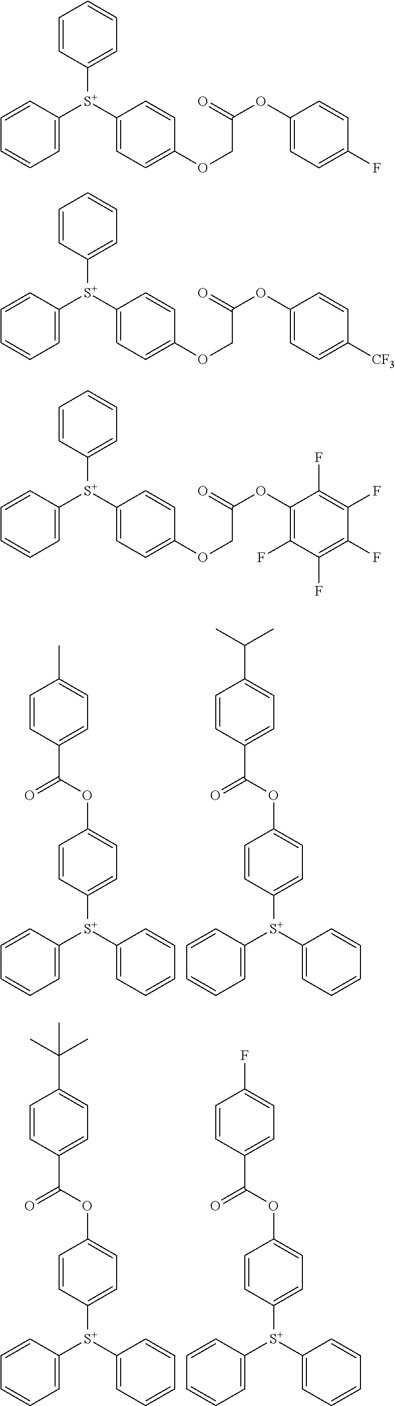

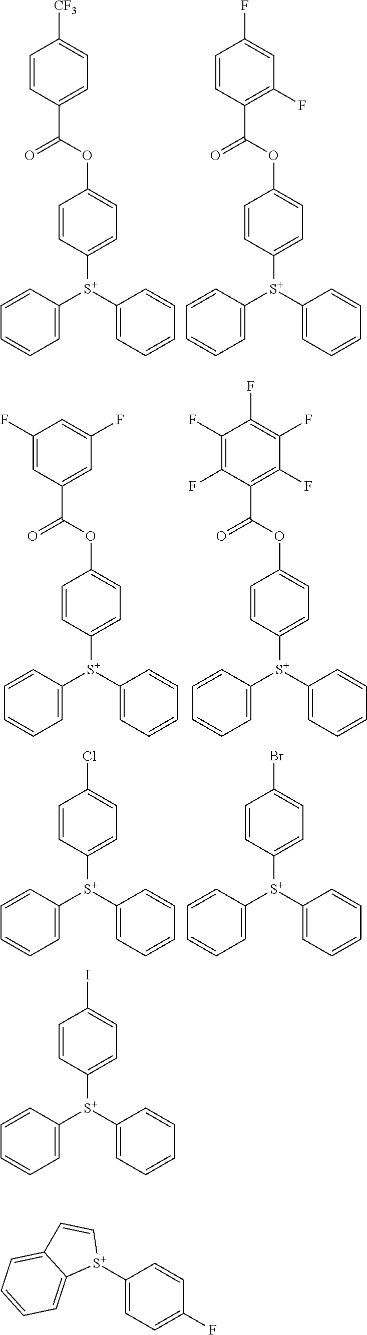

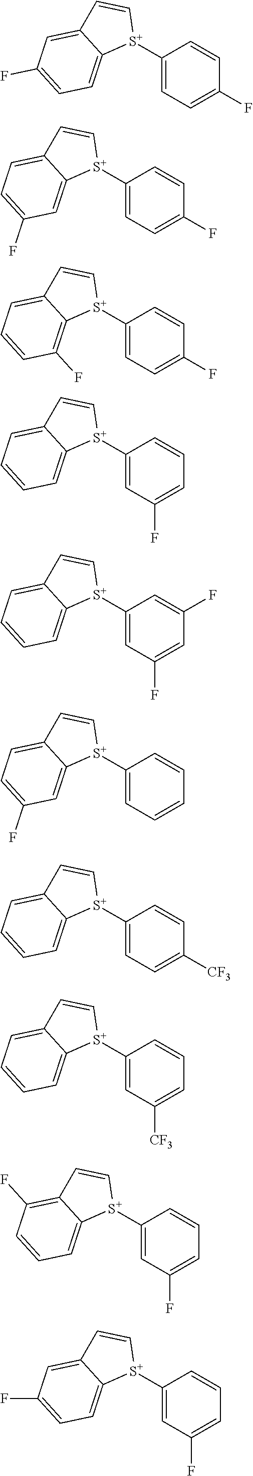

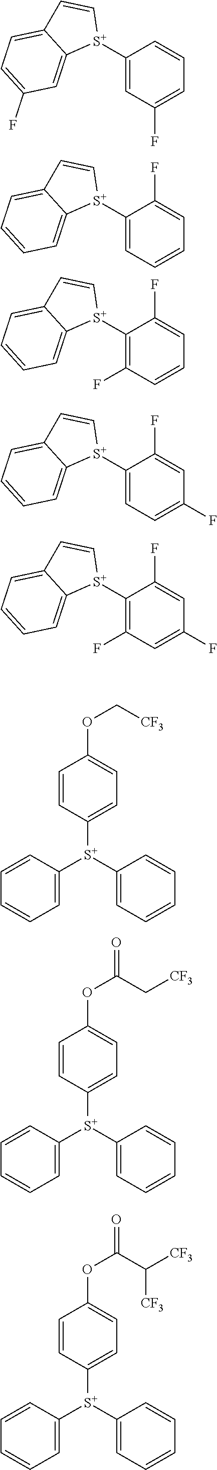

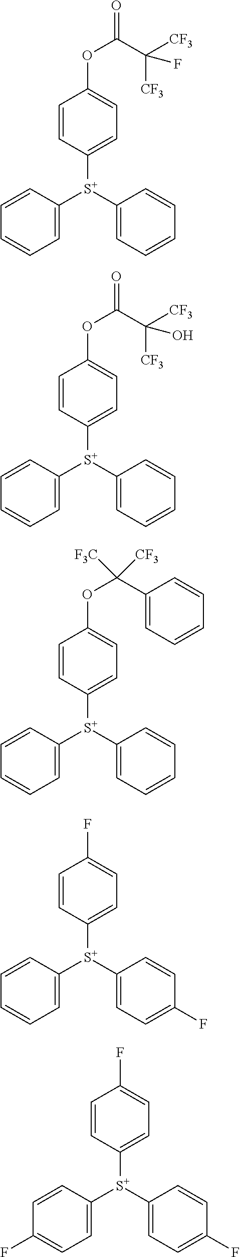

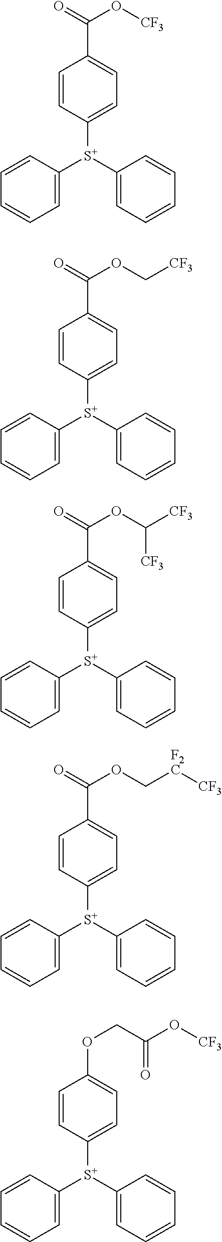

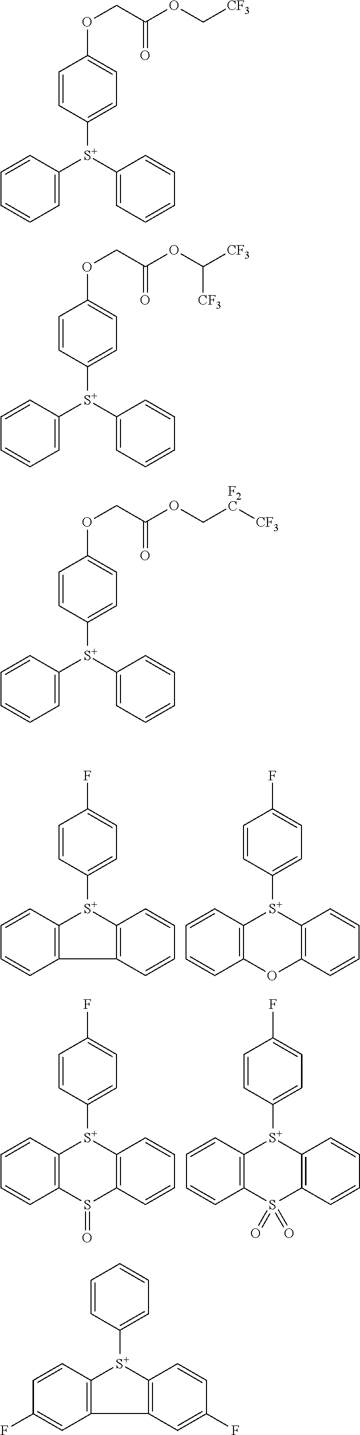

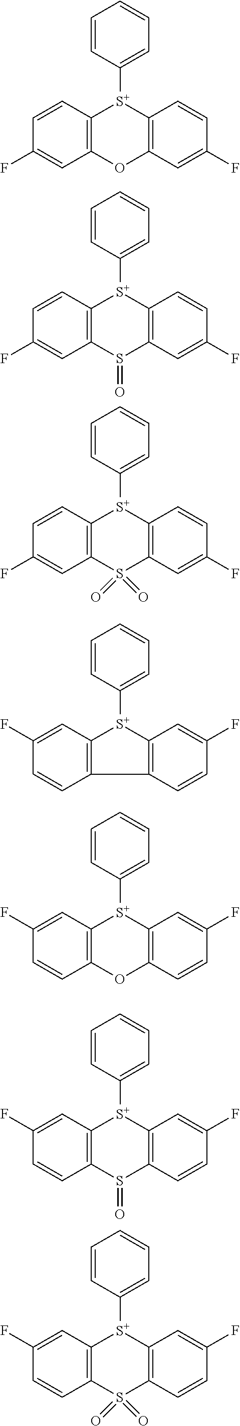

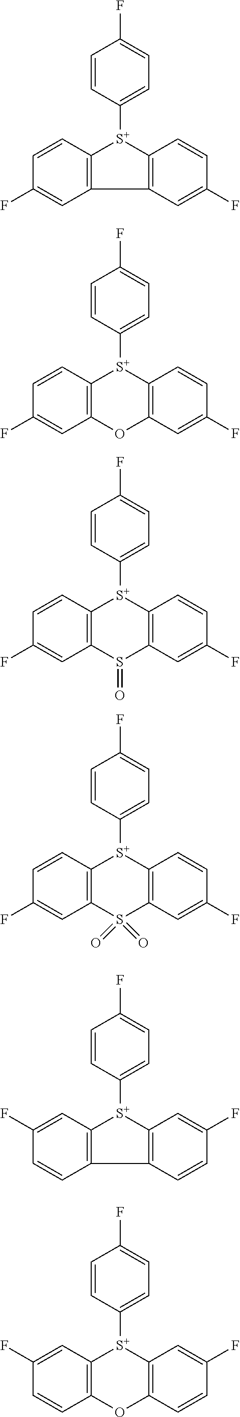

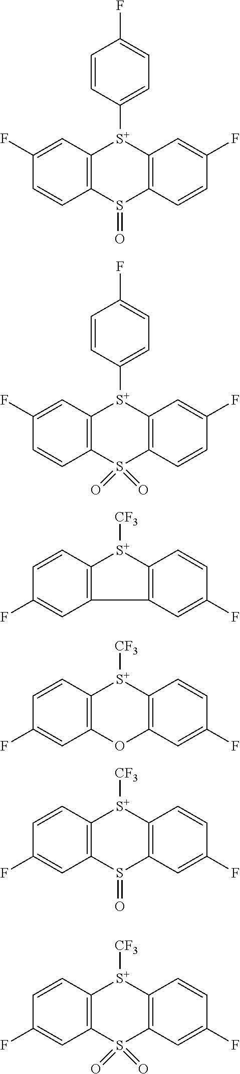

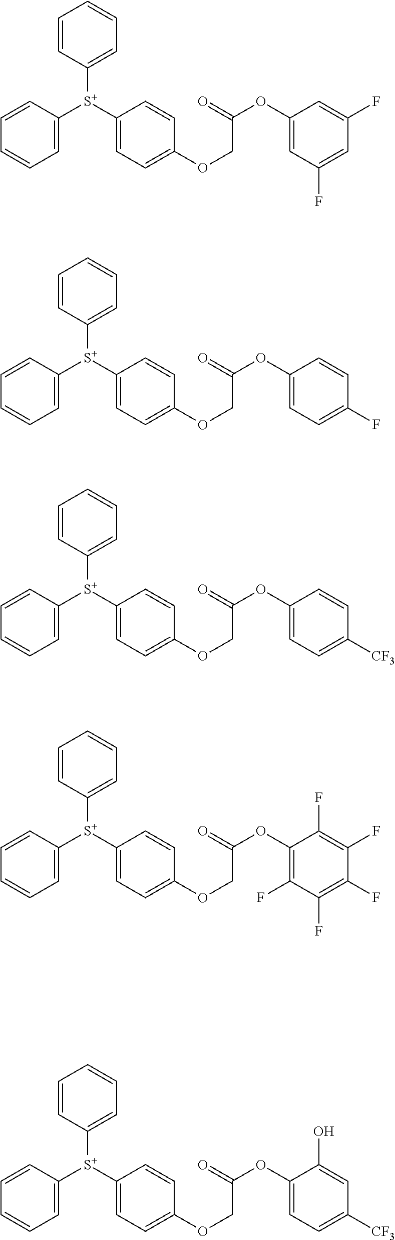

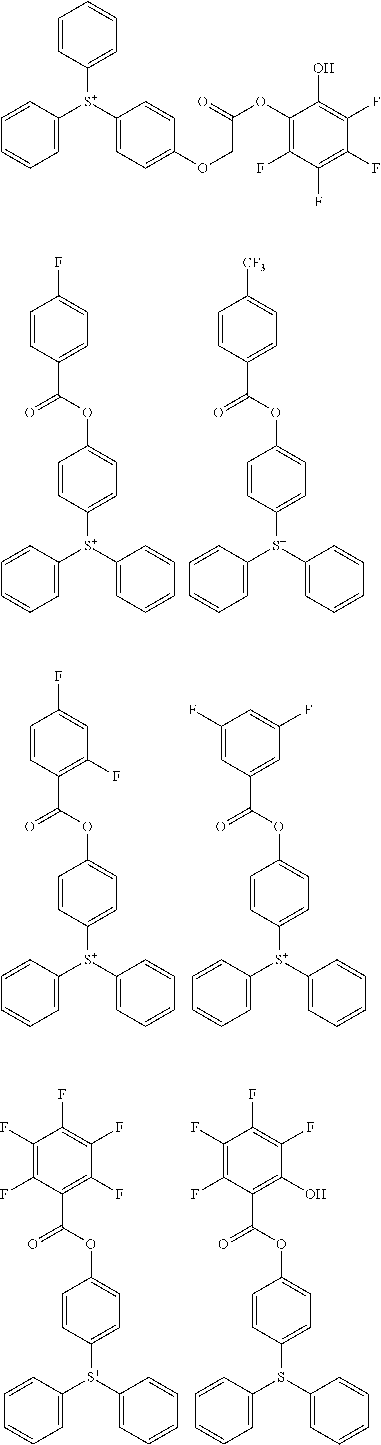

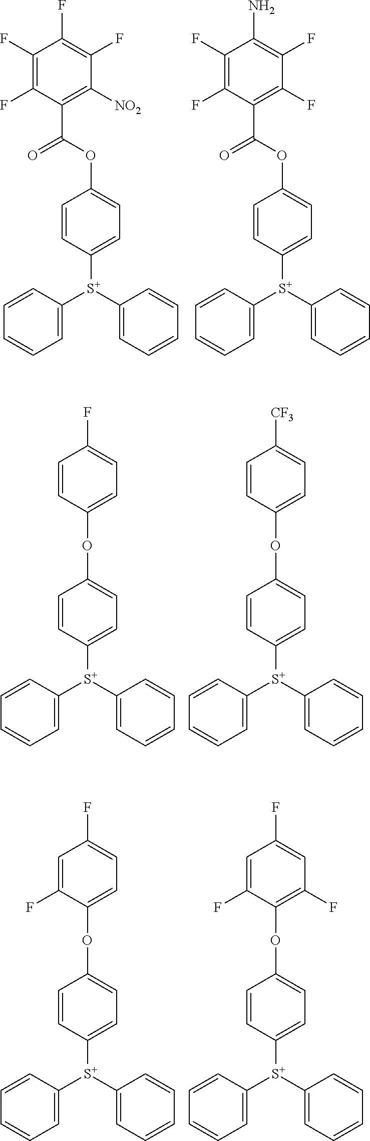

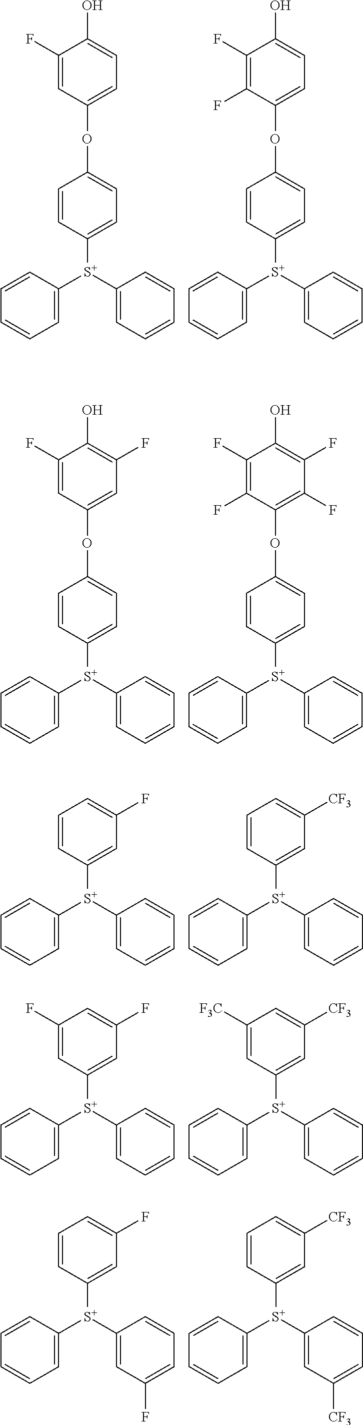

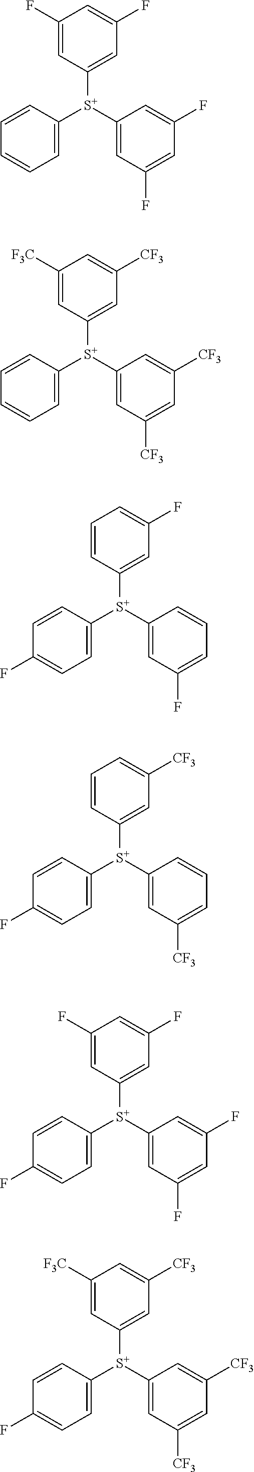

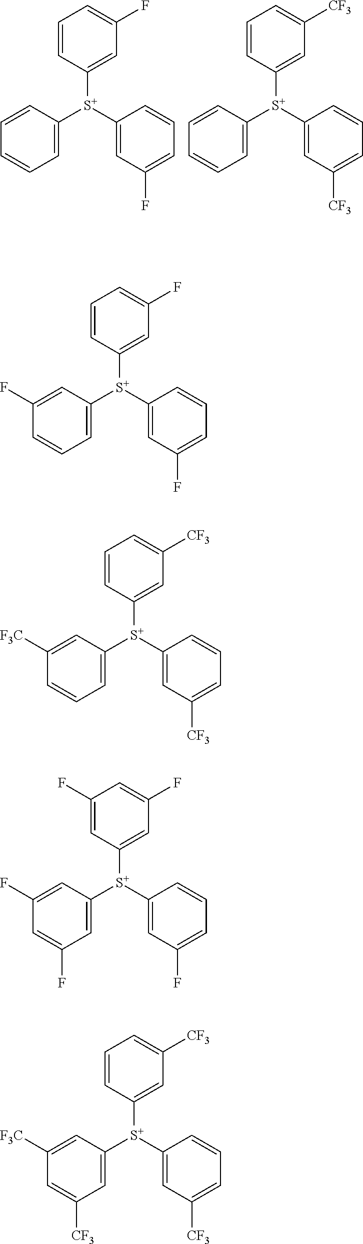

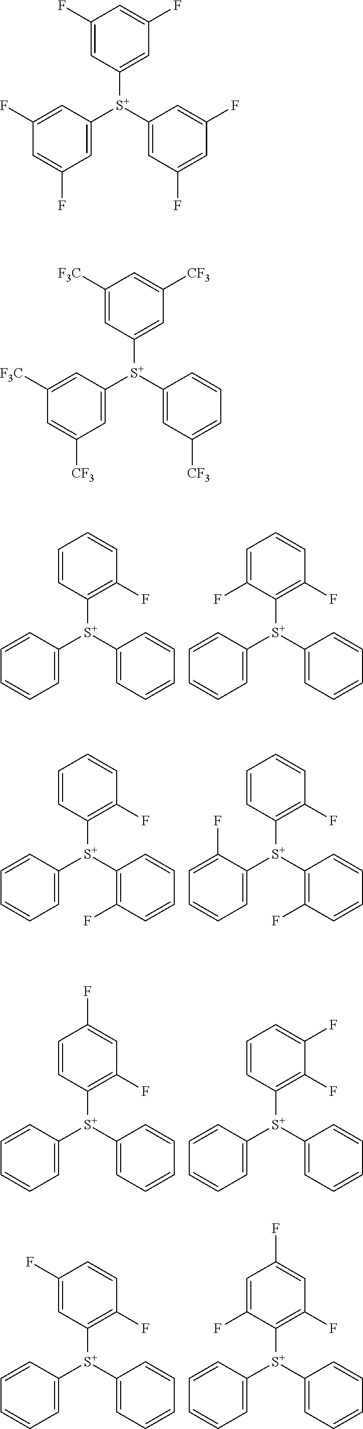

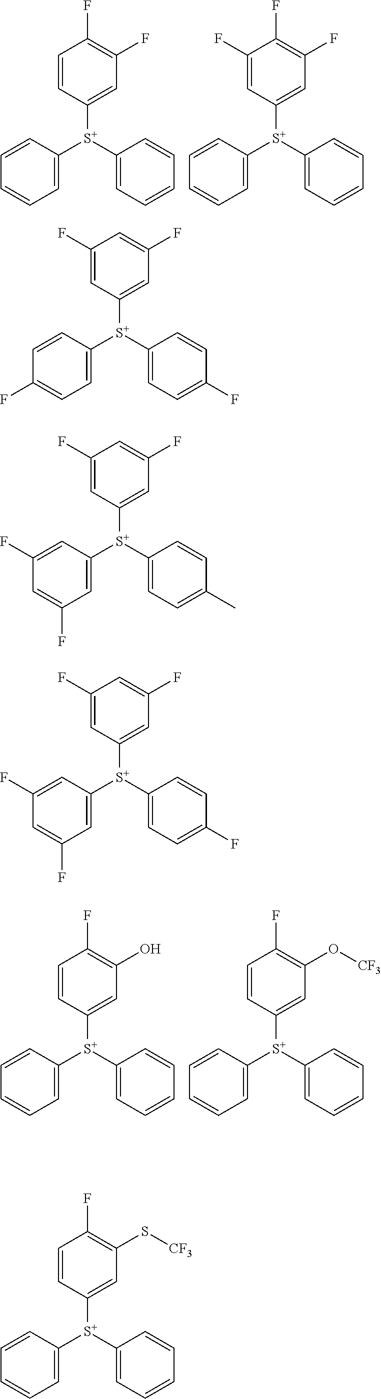

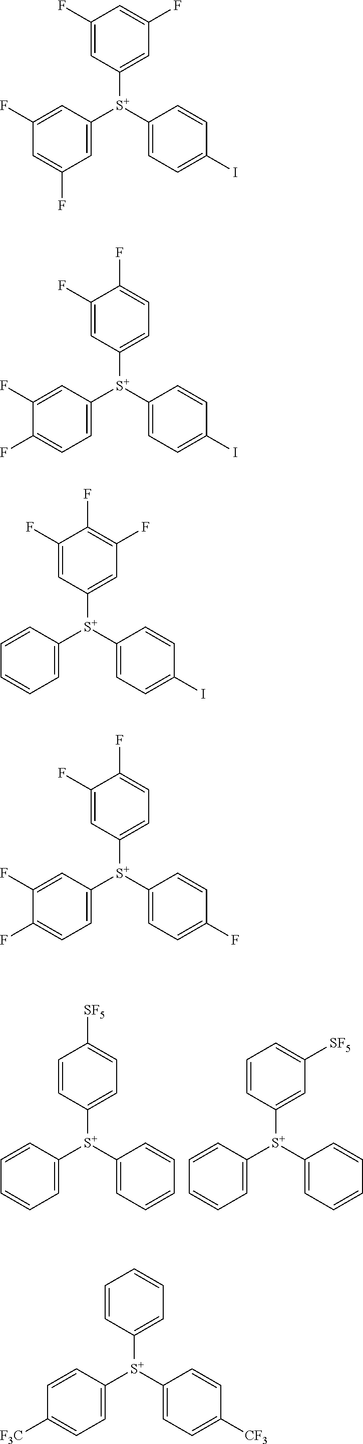

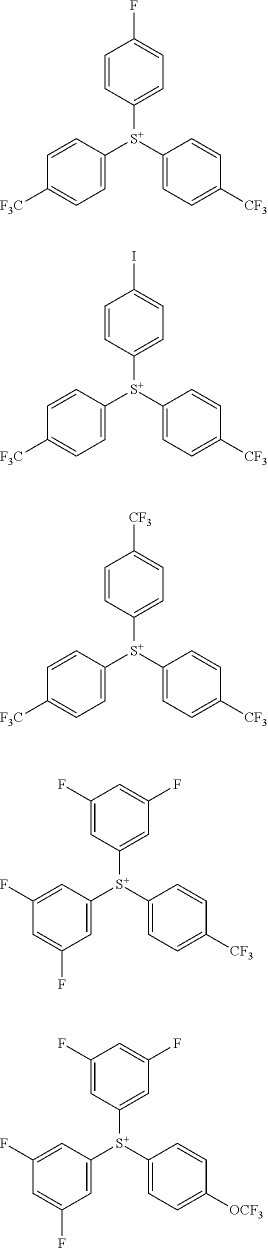

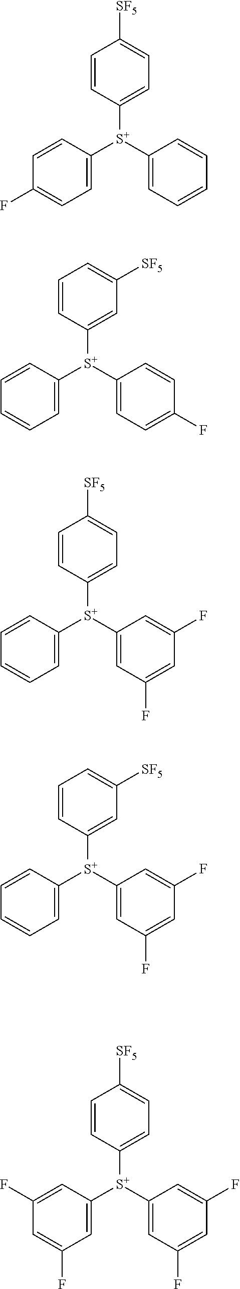

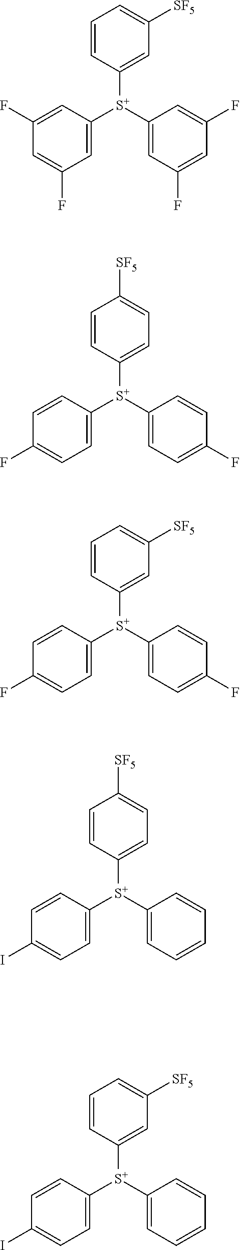

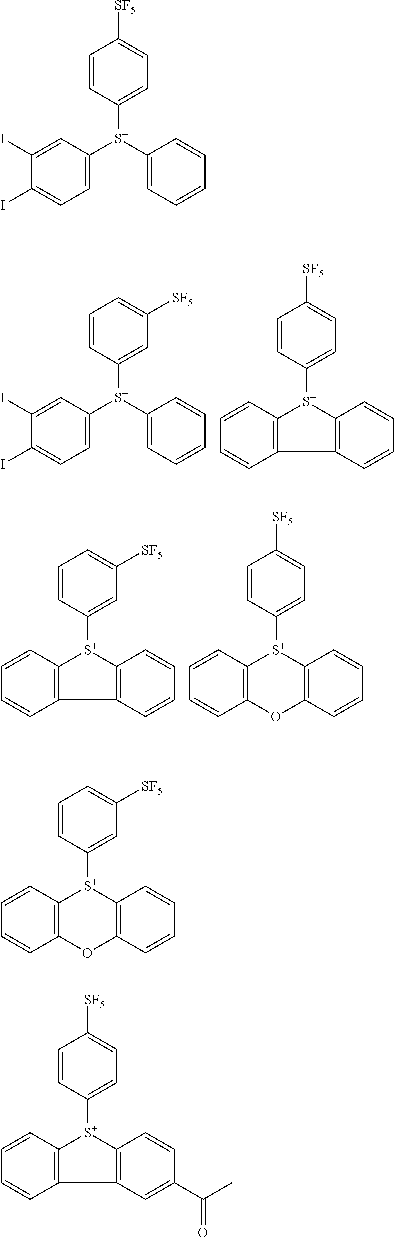

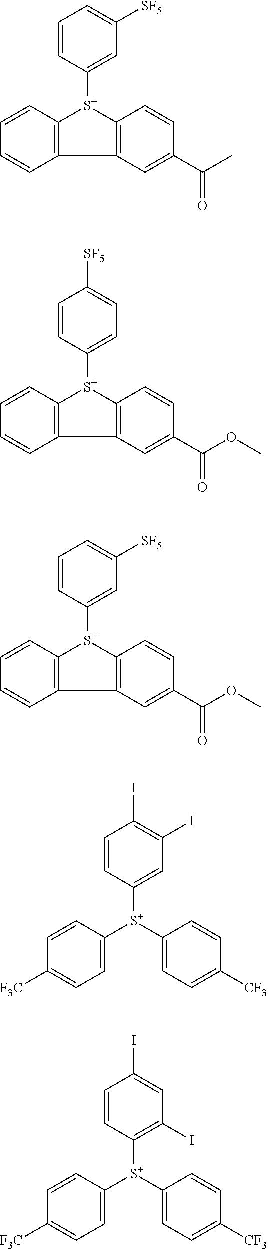

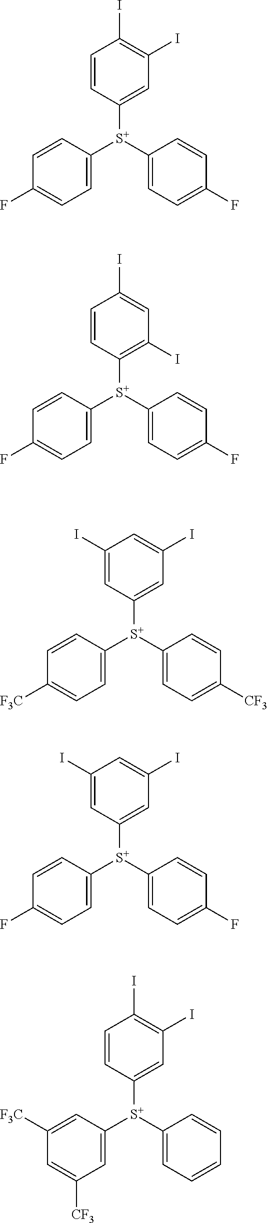

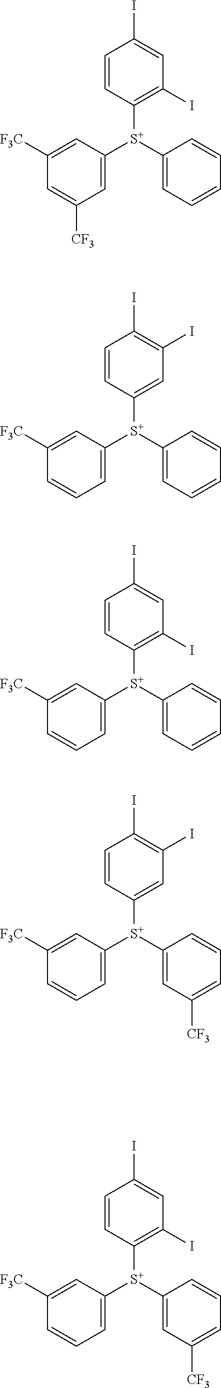

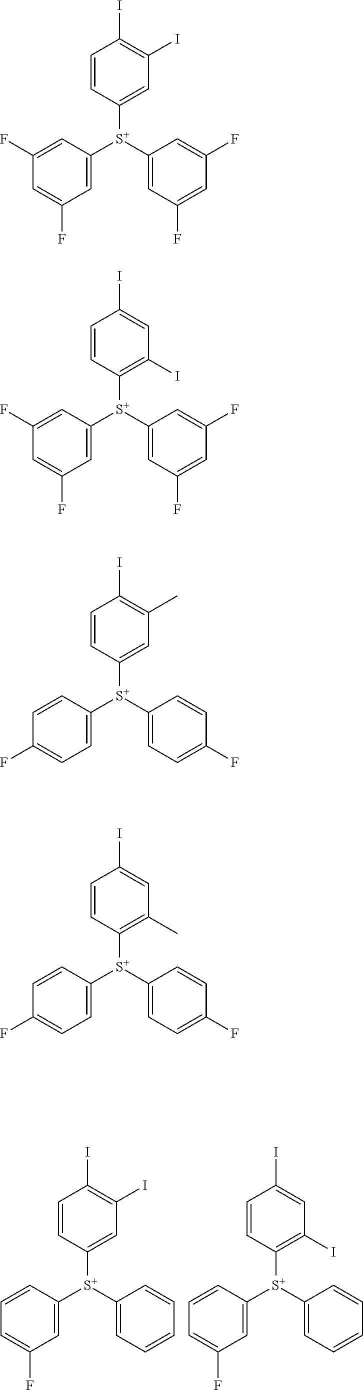

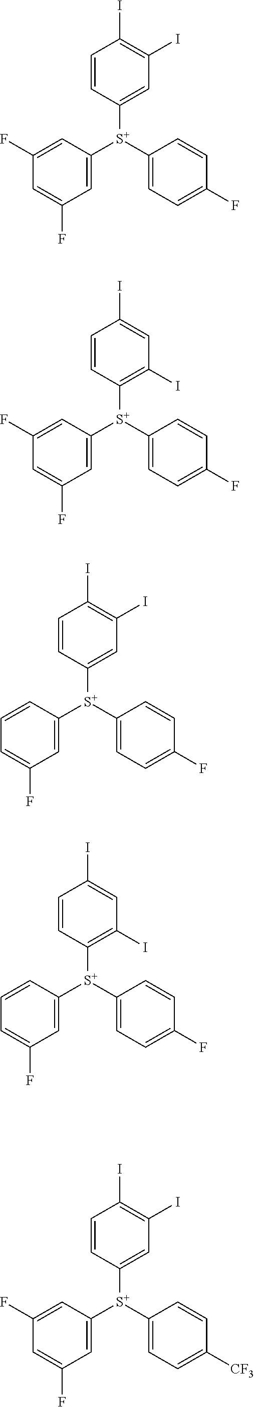

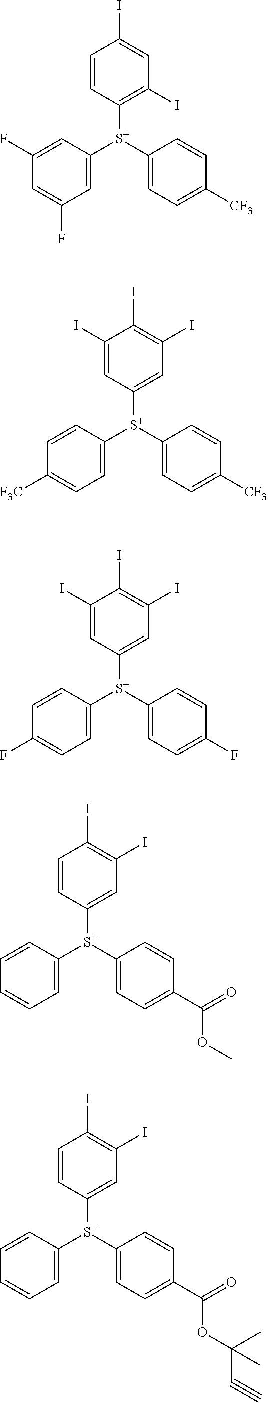

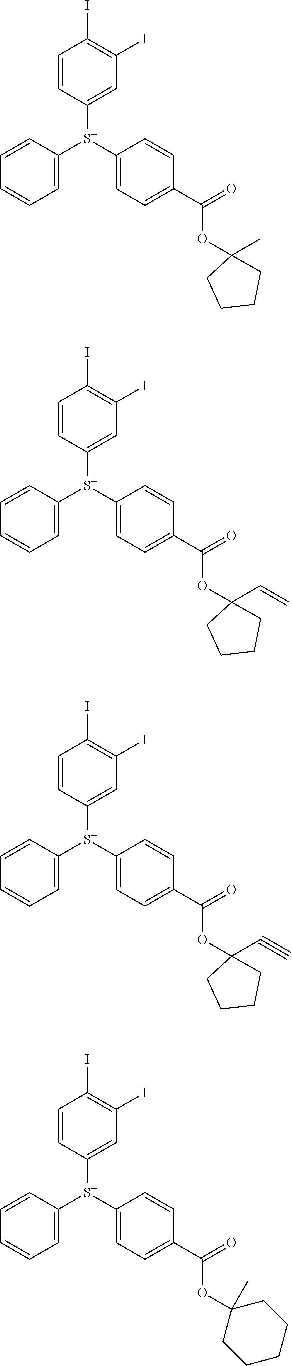

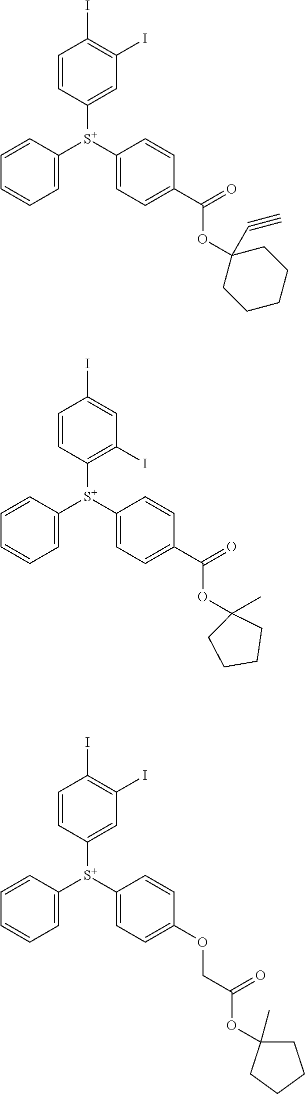

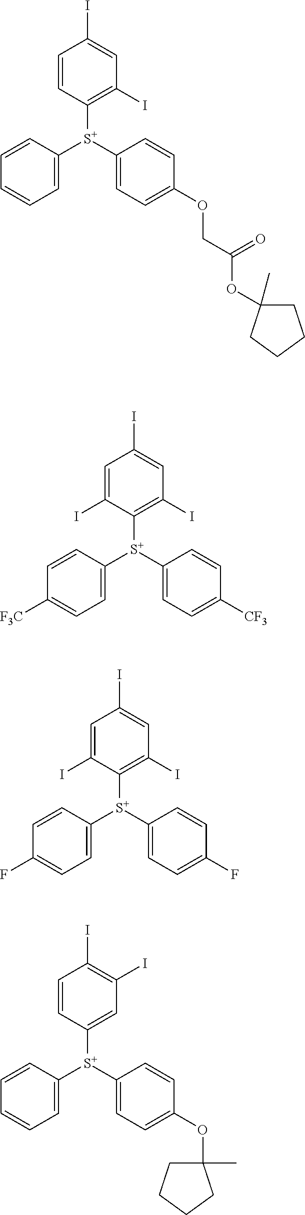

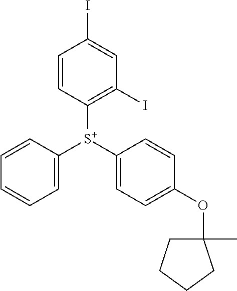

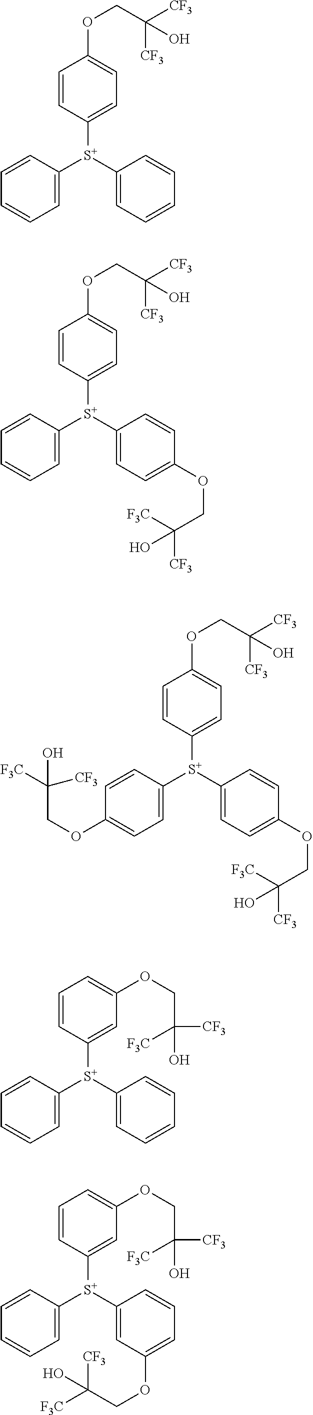

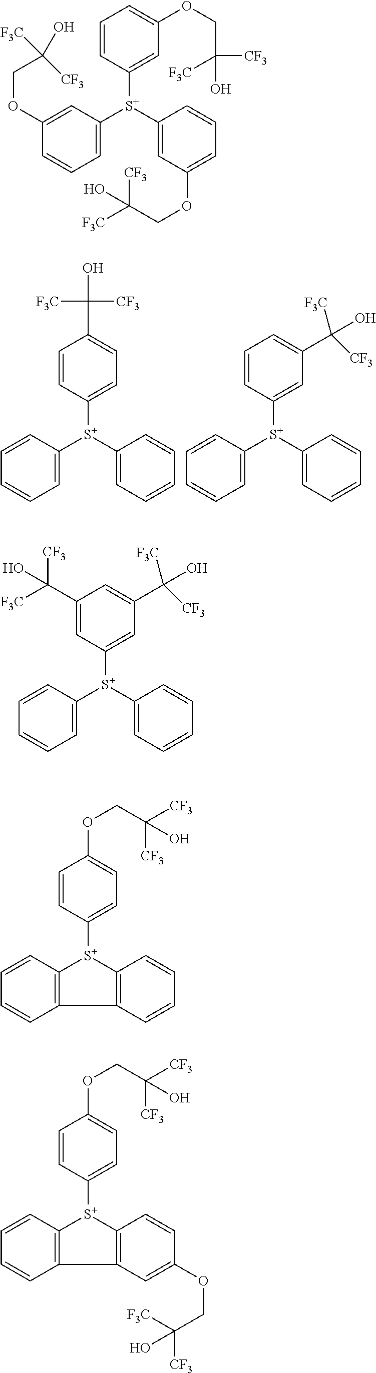

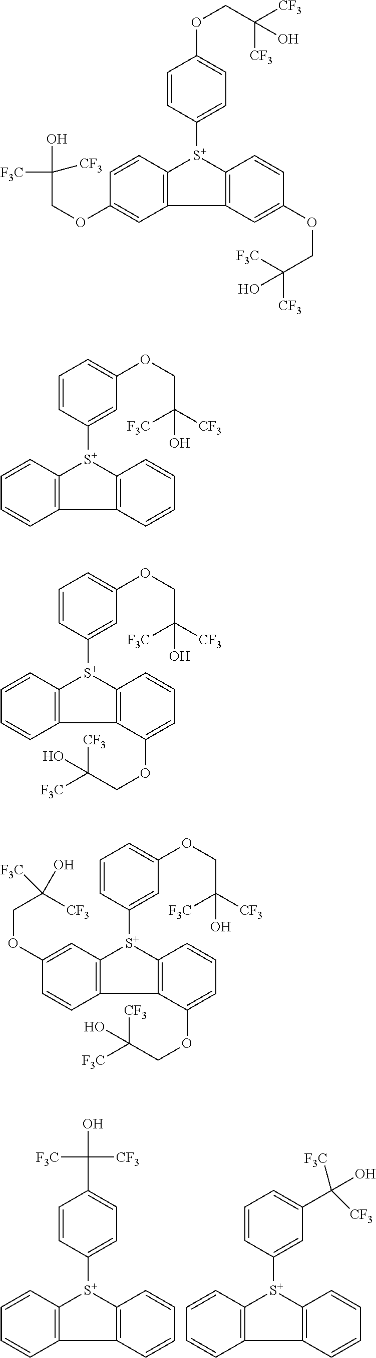

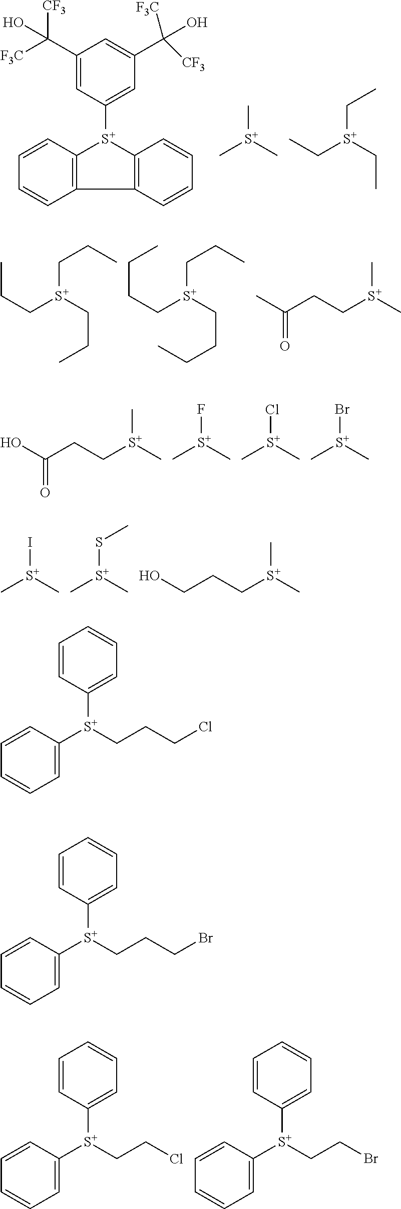

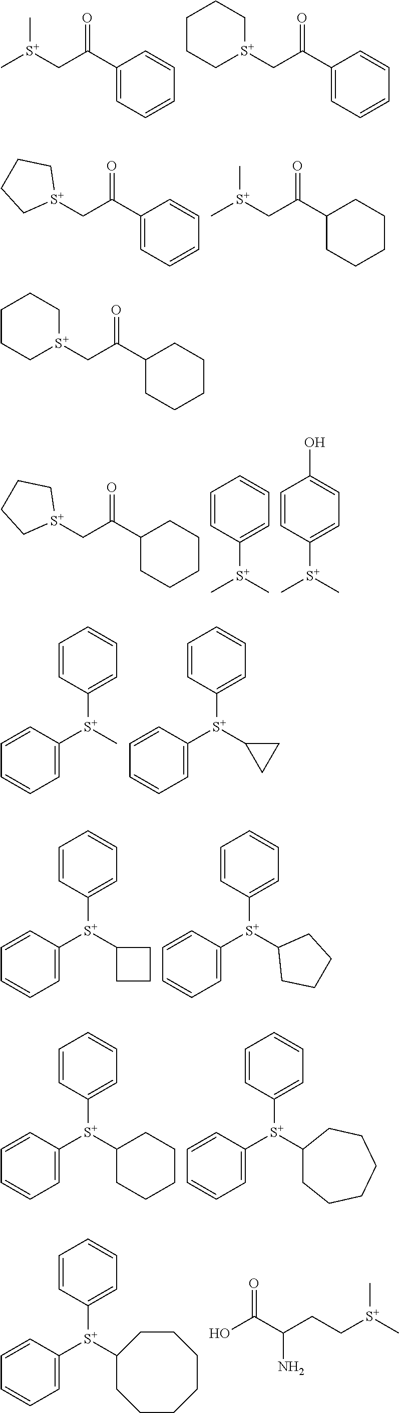

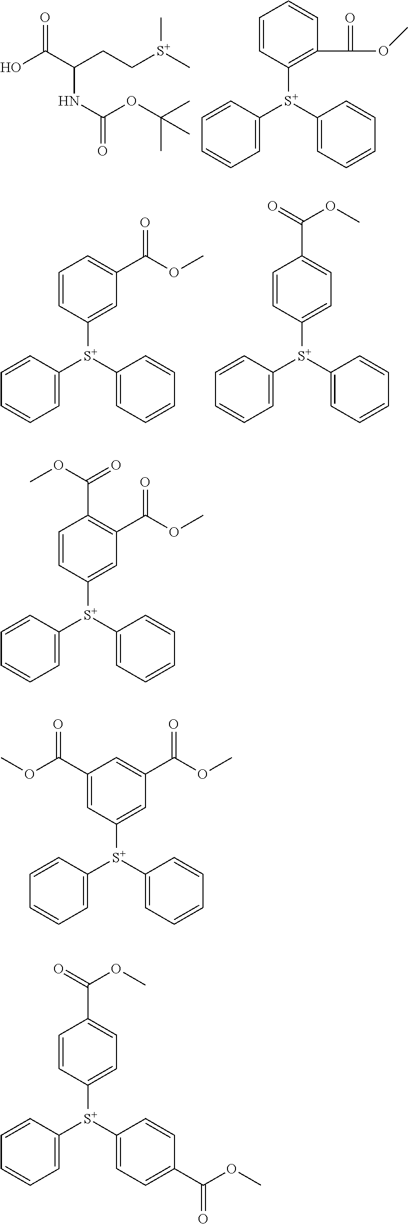

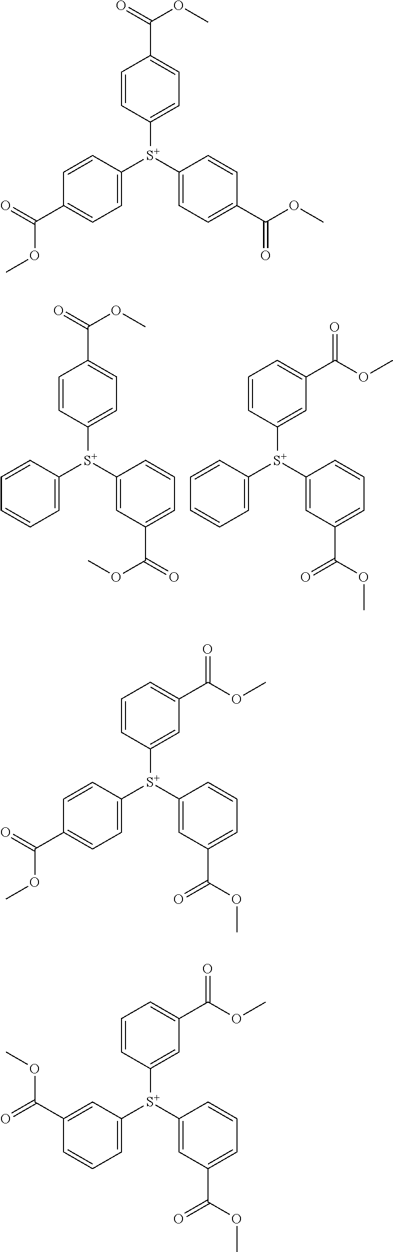

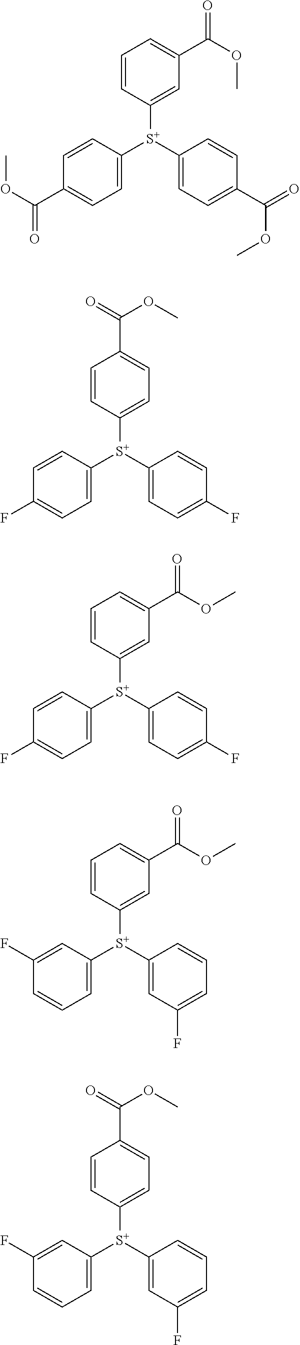

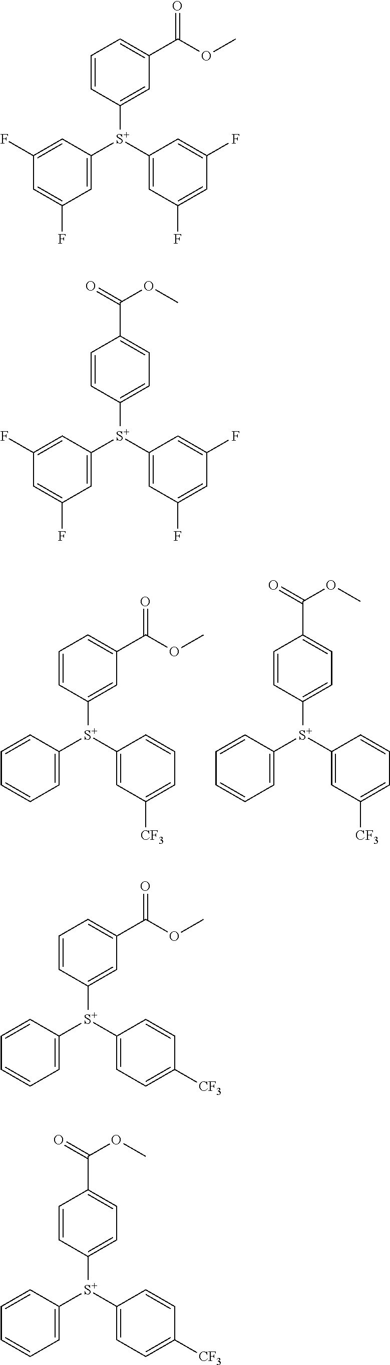

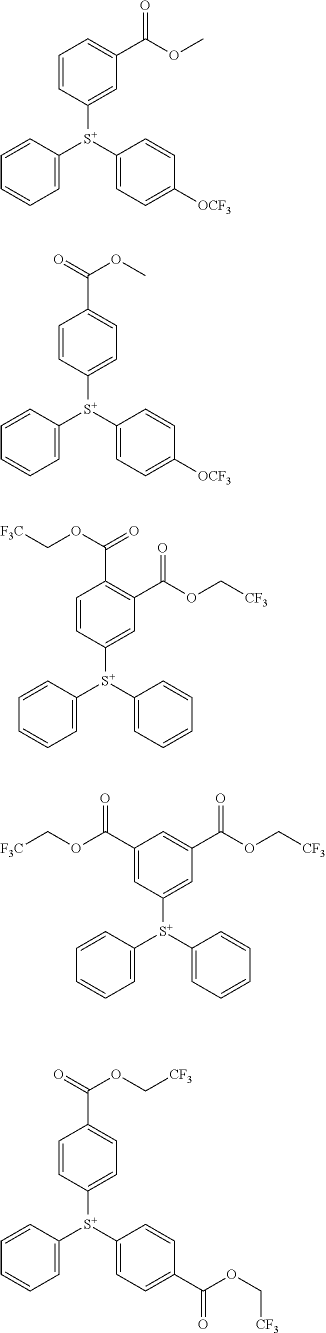

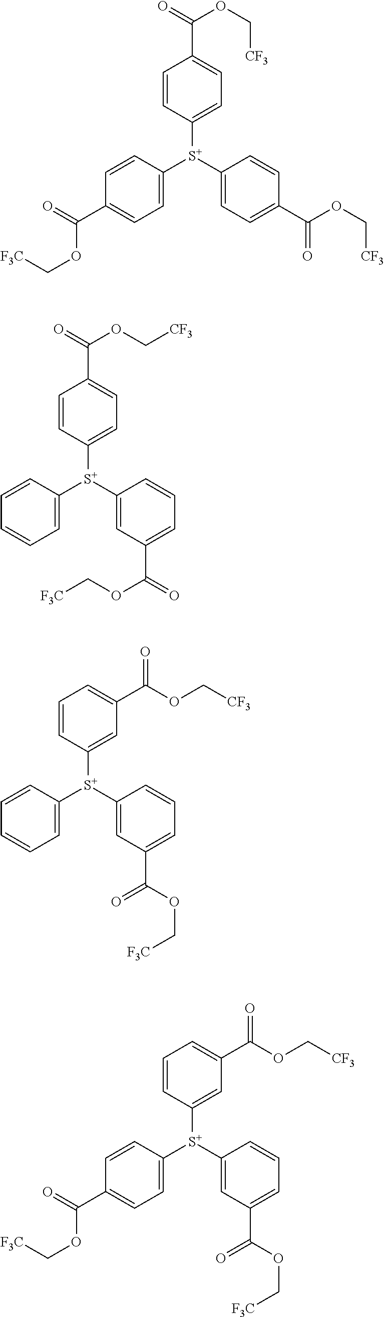

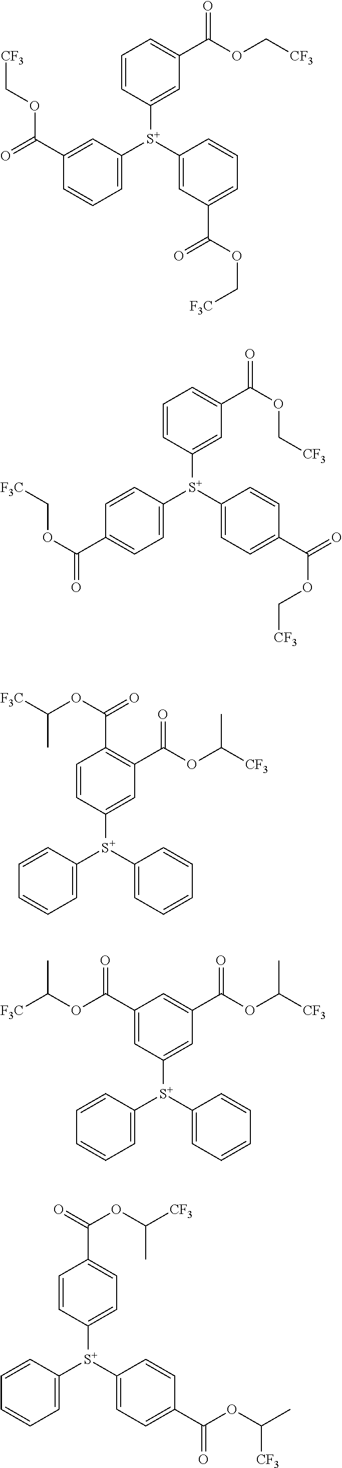

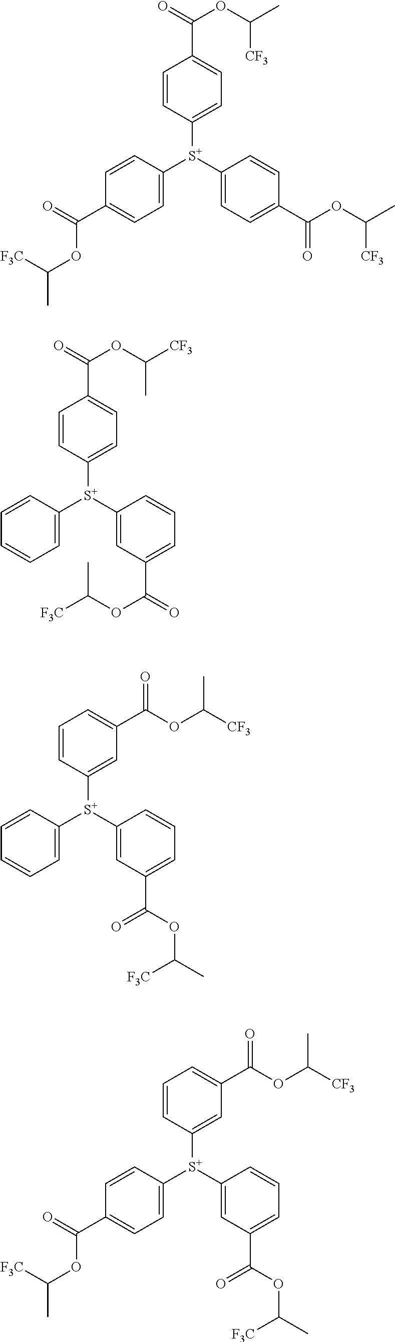

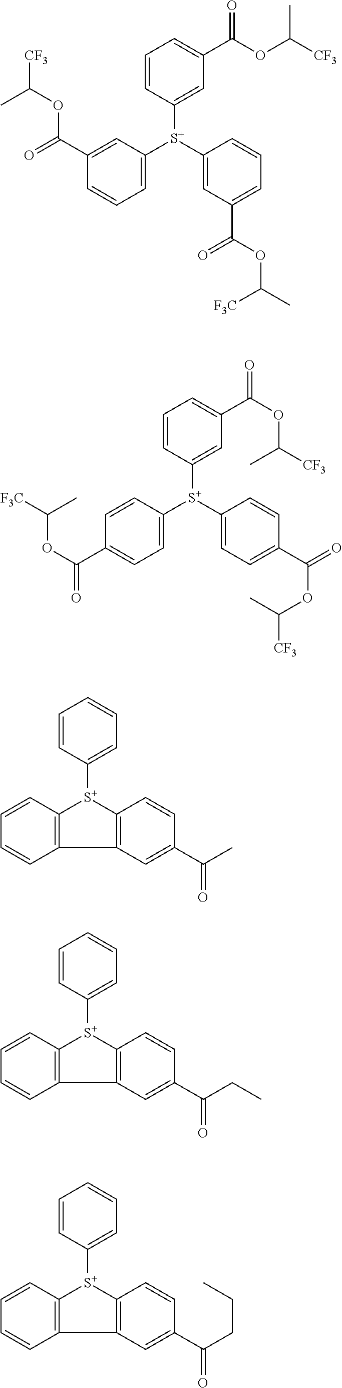

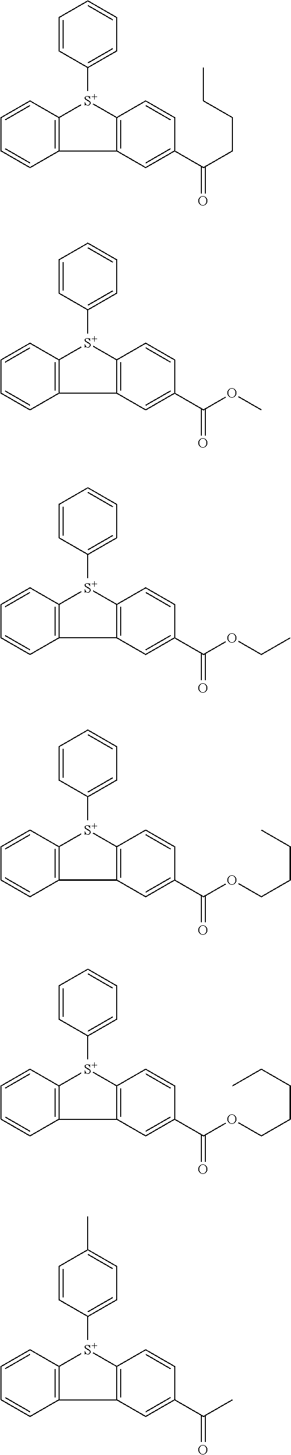

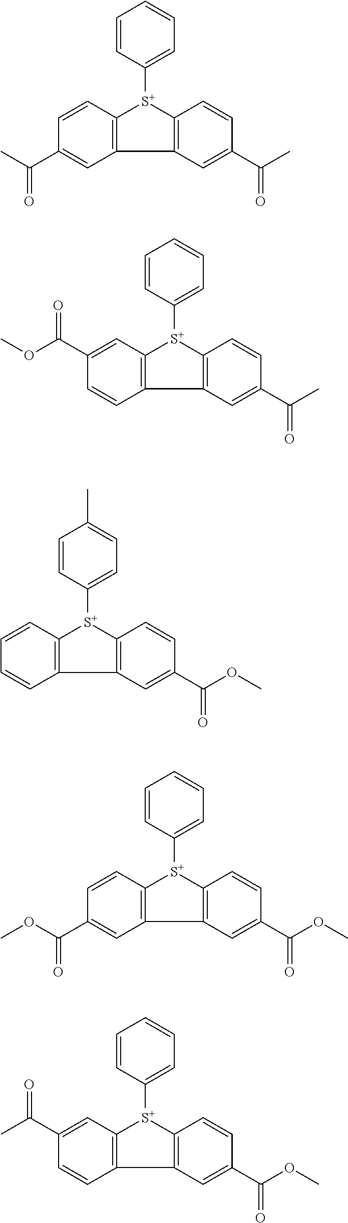

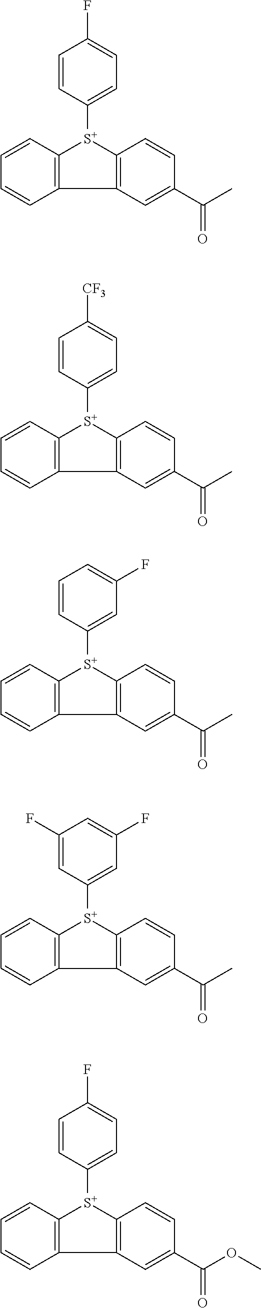

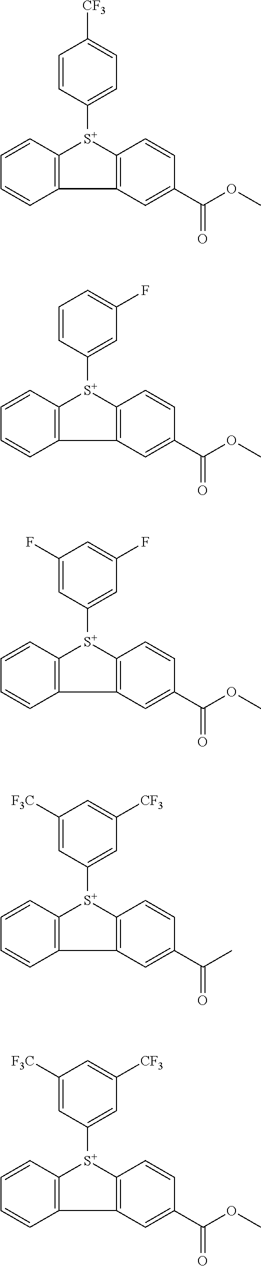

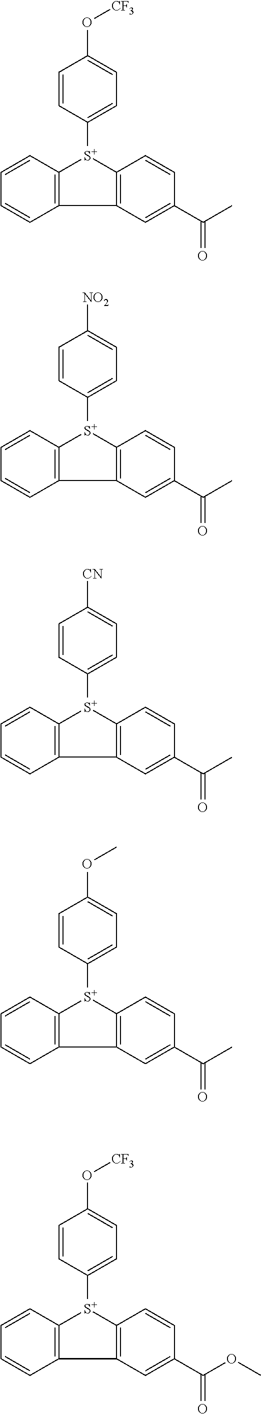

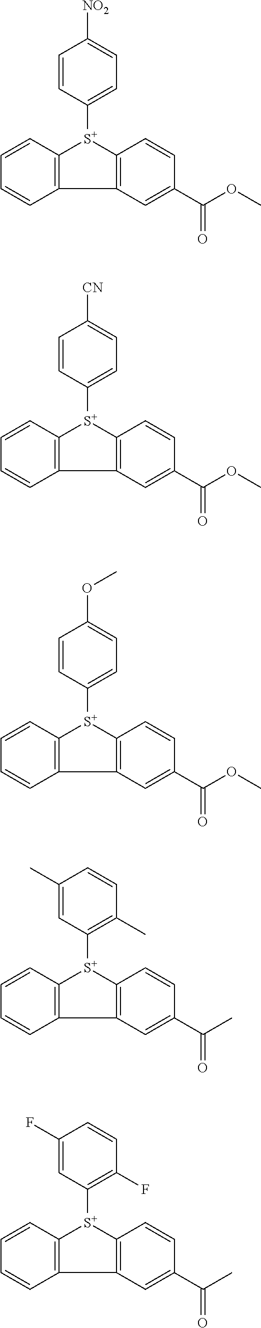

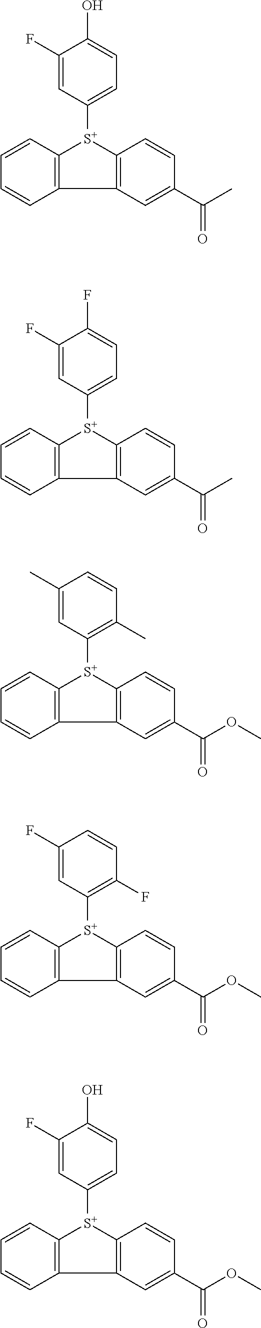

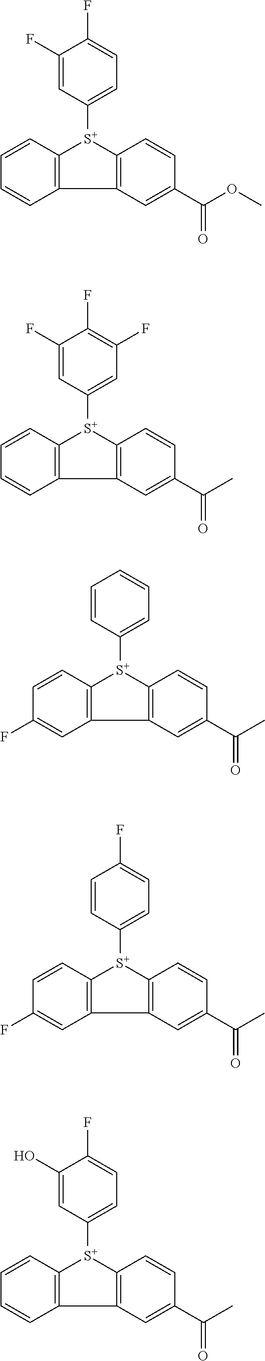

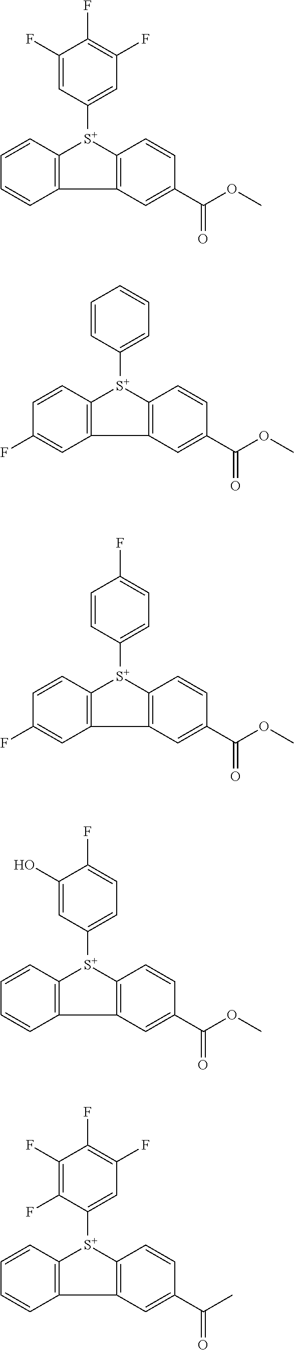

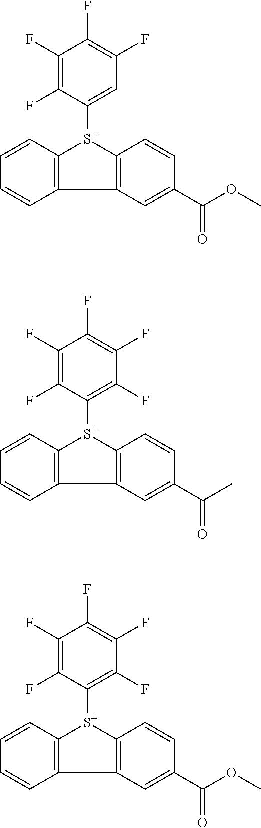

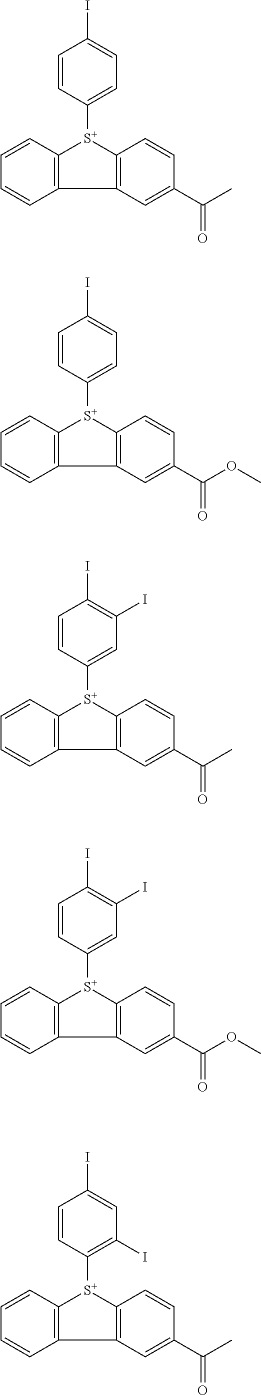

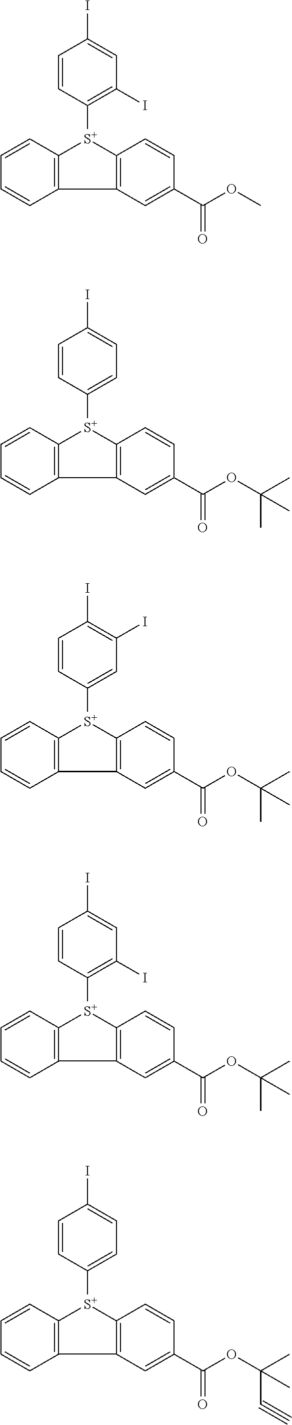

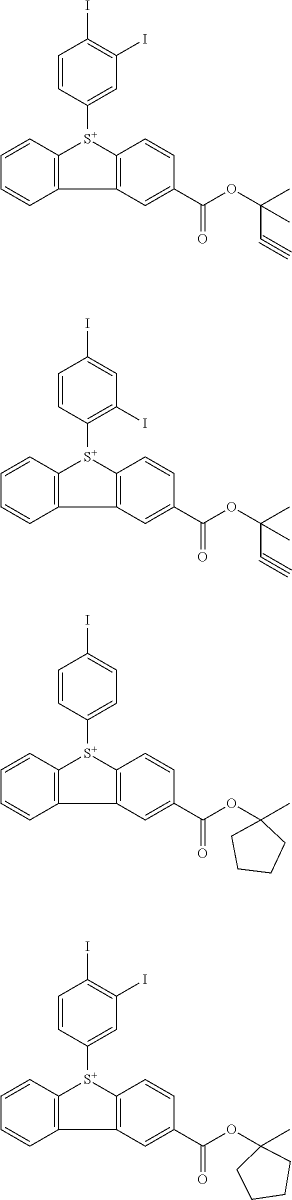

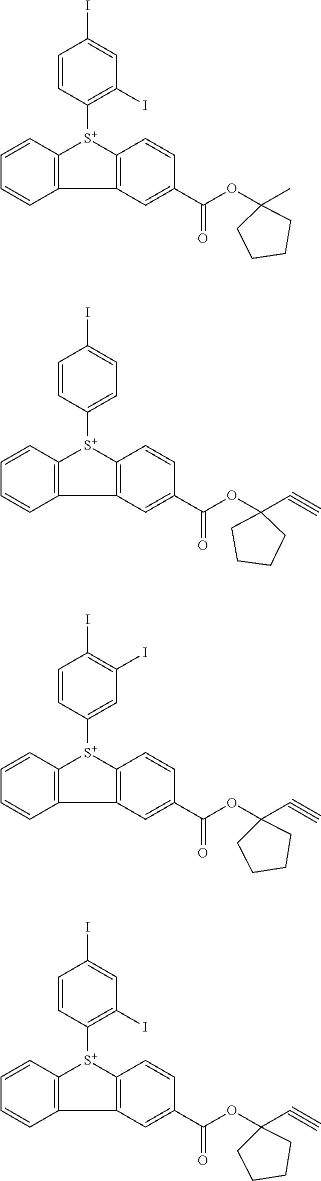

Specific examples of the iodonium cation represented by the general formula (Z-2) include those shown below, but are not limited thereto.

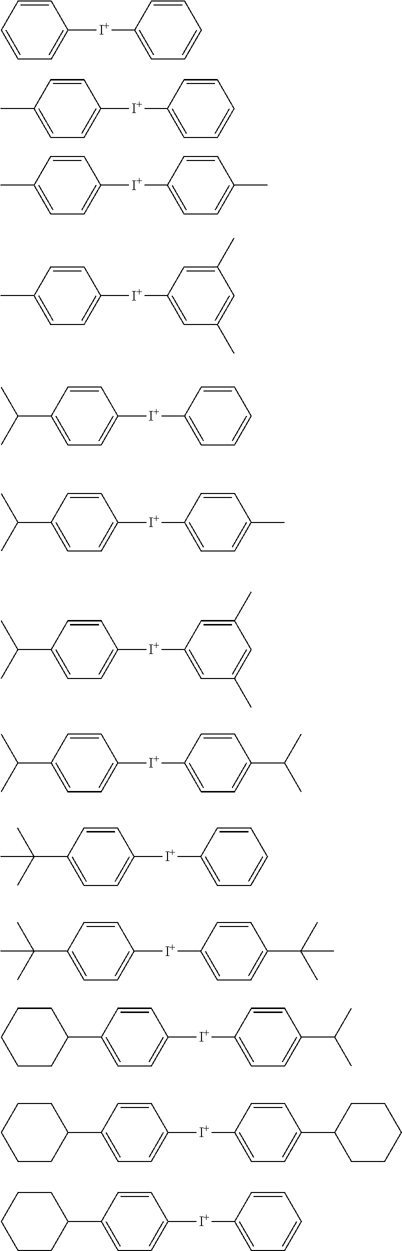

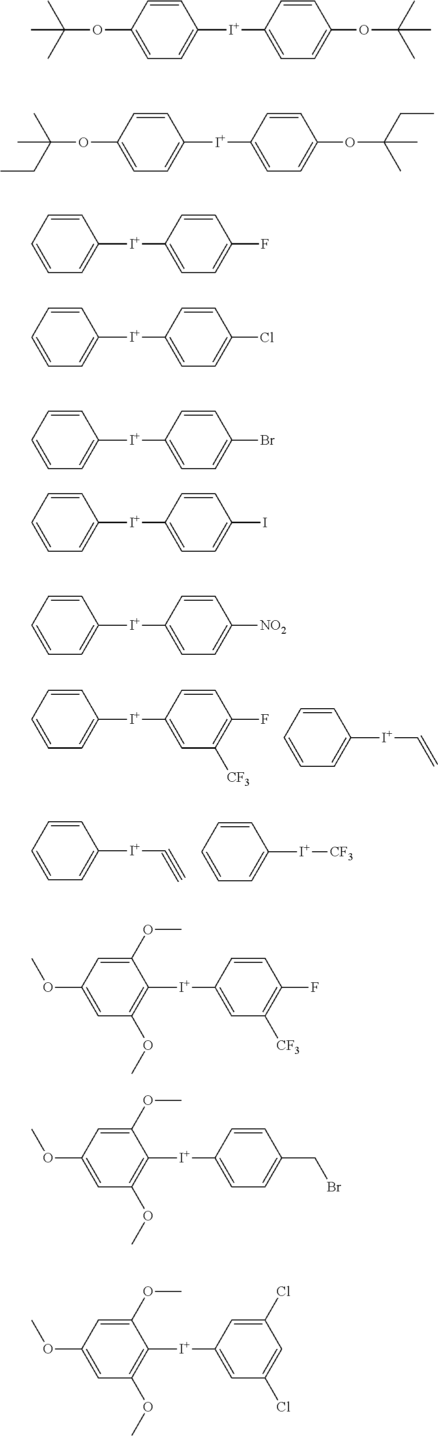

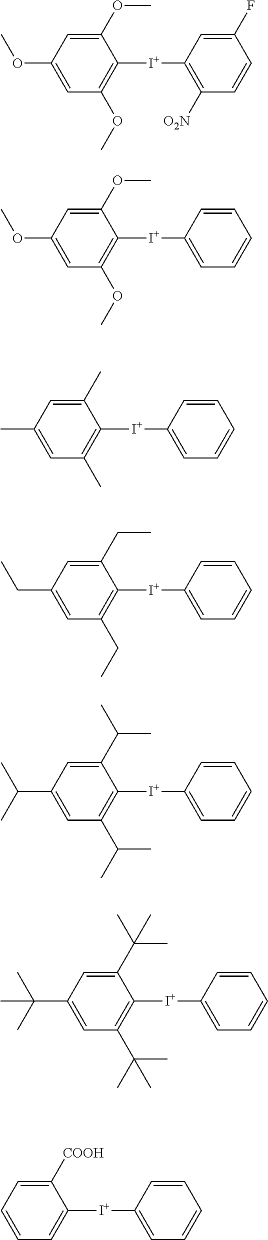

Specific examples of the inventive onium salt include any combination of the above-described anions and the above-described cations.

The inventive onium salt can be synthesized by the publicly known methods. Specifically, the onium salt can be synthesized by the method disclosed in JP7067271B2, but the method for synthesizing the onium salt of the present invention is not limited thereto.

### [Chemically-Amplified Resist Composition]

### [(A) Photoacid Generator]

The inventive chemically-amplified resist composition can contain, as the component (A), a photoacid generator including an onium salt represented by the above general formula (a).

In the inventive chemically-amplified resist composition, the contained amount of the photoacid generator, including the component (a) an onium salt, is preferably 0.1 to 40 parts by mass, more preferably 0.5 to 30 parts by mass, relative to 80 parts by mass of the base polymer that will be described later. When the component (A) content is within this range, it is preferable because the composition exhibits favorable sensitivity and favorable resolution and does not pose a risk of causing a problem of foreign matter after development or during stripping of the resist film. One kind of the photoacid generator may be used, or two or more kinds thereof may be used in combination. Furthermore, an onium salt other than the onium salts represented by the above formula (A) may be used as a photoacid generator in combination.

### [(B) Base Polymer]

The inventive chemically-amplified resist composition preferably further contains, as the component (B), a base polymer having a repeating unit having an acid-labile group, and more preferably contains a base polymer containing a polymer having a repeating unit having an acid-labile group but not having a repeating unit having an aromatic group.

The repeating unit having the acid-labile group is preferably represented by the following formula (B1).

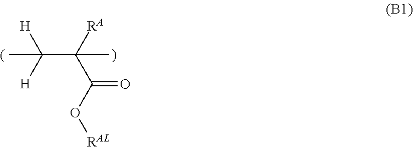

In the formula, R^{A} represents a hydrogen atom or a methyl group, and R^{AL} represents an acid-labile group.

In the general formula (B1), R^{A} represents a hydrogen atom or a methyl group.

In the general formula (B1), R^{AL} represents an acid-labile group. Various types of acid-labile groups can be used. Specific examples thereof include groups represented by the following general formulae (L1) to (L9), tertiary hydrocarbyl groups having 4 to 20, preferably 4 to 15, carbon atoms, trihydrocarbylsilyl groups in which each hydrocarbyl group has 1 to 6 carbon atoms, and hydrocarbyl groups having 4 to 20 carbon atoms and having a carbonyl group, an ether bond, or an ester bond.

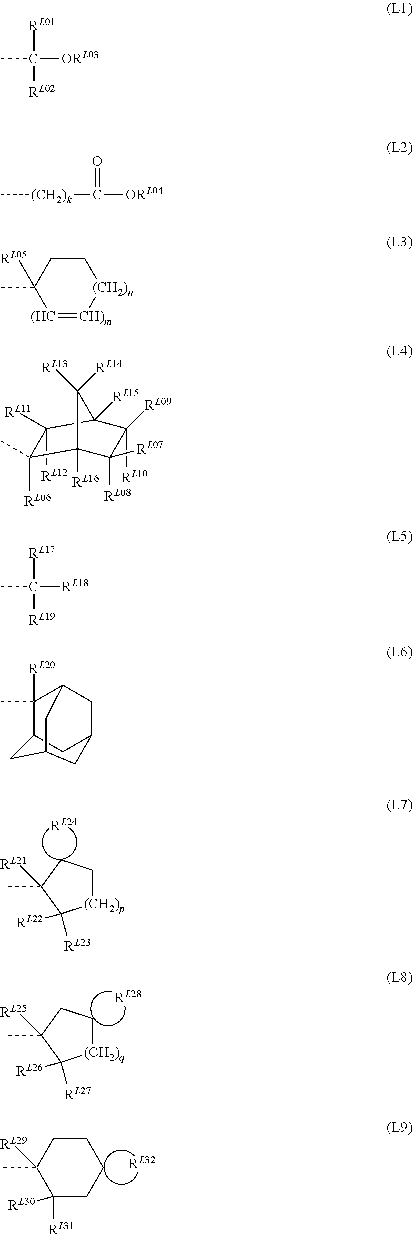

In the formulae, the broken line represents an attachment point.

In the general formula (L1), R^{L01} and R^{L02} each independently represent a hydrogen atom or a saturated hydrocarbyl group having 1 to 18 carbon atoms, preferably 1 to 10 carbon atoms. The saturated hydrocarbyl group may be linear, branched, or cyclic, and specific examples thereof include alkyl groups having 1 to 18 carbon atoms, such as a methyl group, ethyl group, n-propyl group, isopropyl group, n-butyl group, isobutyl group, sec-butyl group, tert-butyl group, 2-ethylhexyl group, and n-octyl group; and saturated cyclic hydrocarbyl groups having 3 to 18 carbon atoms, such as a cyclopentyl group, cyclohexyl group, norbornyl group, tricyclodecanyl group, tetracyclododecanyl group, and adamantyl group.

In the general formula (L1), R^{L03} represents a hydrocarbyl group having 1 to 18 carbon atoms, preferably 1 to 10 carbon atoms, and optionally having a heteroatom. Examples of the heteroatom include an oxygen atom, a nitrogen atom, and a sulfur atom. The hydrocarbyl group may be saturated or unsaturated and may be linear, branched, or cyclic, but is preferably a saturated hydrocarbyl group. Furthermore, part of the hydrogen atoms of the saturated hydrocarbyl group may be substituted with a hydroxy group, a saturated hydrocarbyloxy group having 1 to 8 carbon atoms, an oxo group, an amino group, a saturated hydrocarbylamino group having 1 to 8 carbon atoms, or the like, and part of the —CH₂— groups of the saturated hydrocarbyl group may be substituted with a group having a heteroatom, such as an oxygen atom. Examples of the saturated hydrocarbyl group include the same groups as those exemplified by the saturated hydrocarbyl groups represented by R^{LO1} and R^{L02}. Examples of the substituted saturated hydrocarbyl group include the groups shown below.

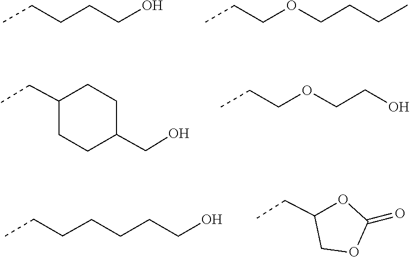

In the formulae, the broken line represents an attachment point.

Any two of R^{L01}, R^{L02}, and R^{L03} may be bonded to each other to form a ring together with the carbon atom or the oxygen atom to which they are bonded. When a ring is formed, the group formed by their bonding is preferably an alkanediyl group having 1 to 18 carbon atoms, preferably 1 to 10 carbon atoms.

In the general formula (L2), R^{L04} represents a tertiary hydrocarbyl group having 4 to 20 carbon atoms, preferably 4 to 15 carbon atoms, a trihydrocarbylsilyl group in which each hydrocarbyl group has 1 to 6 carbon atoms, a saturated hydrocarbyl group having 4 to 20 carbon atoms and having a carbonyl group, an ether bond, or an ester bond, or a group represented by the general formula (L1). “k” represents an integer of 0 to 6.

The tertiary hydrocarbyl group represented by R^{L04} may be branched or cyclic, and specific examples thereof include a tert-butyl group, a tert-pentyl group, a 1,1-diethylpropyl group, a 2-cyclopentylpropan-2-yl group, a 2-cyclohexylpropan-2-yl group, a 2-(bicyclo[2.2.1]heptan-2-yl)propan-2-yl group, a 2-(adamantan-1-yl)propan-2-yl group, a 1-ethylcyclopentyl group, a 1-butylcyclopentyl group, a 1-ethylcyclohexyl group, a 1-butylcyclohexyl group, a 1-ethyl-2-cyclopentenyl group, a 1-ethyl-2-cyclohexenyl group, a 2-methyl-2-adamantyl group, and a 2-ethyl-2-adamantyl group. Specific examples of the trihydrocarbylsilyl group include a trimethylsilyl group, a triethylsilyl group, and a dimethyl-tert-butylsilyl group. Specific examples of the saturated hydrocarbyl group having a carbonyl group, an ether bond, or an ester bond include a 3-oxocyclohexyl group, a 4-methyl-2-oxooxan-4-yl group, and a 5-methyl-2-oxooxolan-5-yl group.

In the general formula (L3), R^{L05} represents a saturated hydrocarbyl group having 1 to 8 carbon atoms and optionally having a heteroatom, or an aryl group having 6 to 20 carbon atoms, optionally being substituted, and optionally having a heteroatom. The saturated hydrocarbyl group may be linear, branched, or cyclic, and specific examples thereof include: alkyl groups, such as a methyl group, an ethyl group, an n-propyl group, an isopropyl group, an n-butyl group, an isobutyl group, a sec-butyl group, a tert-butyl group, an n-pentyl group, a tert-pentyl group, and an n-hexyl group; and saturated cyclic hydrocarbyl groups, such as a cyclopentyl group and a cyclohexyl group. In addition, part of the hydrogen atoms of the saturated hydrocarbyl group may be substituted with a hydroxy group, a saturated hydrocarbyloxy group having 1 to 8 carbon atoms, a carboxy group, a saturated hydrocarbyloxycarbonyl group having 1 to 8 carbon atoms, an oxo group, an amino group, a saturated hydrocarbylamino group having 1 to 8 carbon atoms, a cyano group, a mercapto group, a saturated hydrocarbylthio group having 1 to 8 carbon atoms, a sulfo group, etc. Specific examples of the aryl group include a phenyl group, a methylphenyl group, a naphthyl group, an anthryl group, a phenanthryl group, and a pyrenyl group. In addition, part of the hydrogen atoms of the aryl group may be substituted with a hydroxy group, a saturated hydrocarbyloxy group having 1 to 8 carbon atoms, a carboxy group, a saturated hydrocarbylcarbonyl group having 1 to 8 carbon atoms, an oxo group, an amino group, a saturated hydrocarbylamino group having 1 to 8 carbon atoms, a cyano group, a mercapto group, a saturated hydrocarbylthio group having 1 to 8 carbon atoms, a sulfo group, or the like.

In the general formula (L3), “m” represents 0 or 1, “n” represents an integer of 0 to 3, and “m” and “n” satisfy 2m+n=2 or 3.

In the general formula (L4), R^{L06} represents a hydrocarbyl group having 1 to 8 carbon atoms and optionally having a heteroatom, or an aryl group having 6 to 20 carbon atoms and optionally having a heteroatom. Specific examples of the saturated hydrocarbyl group and the aryl group include the same as those exemplified as the saturated hydrocarbyl group and aryl group, which are represented by R^{L05}, respectively. In addition, part of the hydrogen atoms of the saturated hydrocarbyl group and the aryl group may be substituted with a hydroxy group, a saturated hydrocarbyloxy group having 1 to 8 carbon atoms, a carboxy group, a saturated hydrocarbylcarbonyl group having 1 to 8 carbon atoms, an oxo group, an amino group, a saturated hydrocarbylamino group having 1 to 8 carbon atoms, a cyano group, a mercapto group, a saturated hydrocarbylthio group having 1 to 8 carbon atoms, a sulfo group, or the like.

In the general formula (L4), R^{L07} to R^{L16} each independently represent a hydrogen atom or a hydrocarbyl group having 1 to 15 carbon atoms. The hydrocarbyl group may be saturated or unsaturated and may be linear, branched, or cyclic, but is preferably a saturated hydrocarbyl group. Specific examples of the hydrocarbyl group include: alkyl groups, such as a methyl group, an ethyl group, an n-propyl group, an isopropyl group, an n-butyl group, a sec-butyl group, a tert-butyl group, an n-pentyl group, a tert-pentyl group, an n-hexyl group, an n-octyl group, an n-nonyl group, and an n-decyl; and saturated cyclic hydrocarbyl groups, such as a cyclopentyl group, a cyclohexyl group, a cyclopentylmethyl group, a cyclopentylethyl group, a cyclopentylbutyl group, a cyclohexylmethyl group, a cyclohexylethyl group, and a cyclohexylbutyl group. In addition, part of the hydrogen atoms of the hydrocarbyl groups may be substituted with a hydroxy group, a saturated hydrocarbyloxy group having 1 to 8 carbon atoms, a carboxy group, a saturated hydrocarbyloxycarbonyl group having 1 to 8 carbon atoms, an oxo group, an amino group, a saturated hydrocarbylamino group having 1 to 8 carbon atoms, a cyano group, a mercapto group, a saturated hydrocarbylthio group having 1 to 8 carbon atoms, a sulfo group, or the like. Two selected from these R^{L07} to R^{L16} may be bonded to each other to form a ring together with the carbon atoms to which they are bonded (for example, R^{L07} and R^{L08}, R^{L07} and R^{L09}, R^{L08} and R^{L10}, R^{L09} and R^{L10}, R^{L11} and R^{L12}, R^{L13} and R^{L14}, etc.). In such cases, the group participating in the ring formation is a hydrocarbylene group having 1 to 15 carbon atoms. Specific examples of the hydrocarbylene group include hydrocarbyl groups in which one hydrogen atom has been removed from the hydrocarbyl groups exemplified above. Furthermore, among R^{L07} to R^{L16}, those bonded to adjacent carbon atoms may be bonded to each other without any intervening to form a double bond (for example, R^{L07} and R^{L09}, R^{L09} and R^{L15}, R^{L13} and R^{L15}, etc.).

In the general formula (L5), R^{L17} to R^{L19} each independently represent a saturated hydrocarbyl group having 1 to 15 carbon atoms and optionally having a heteroatom, or an aryl group having 6 to 15 carbon atoms and optionally having a heteroatom. The saturated hydrocarbyl group may be linear, branched, or cyclic, and specific examples thereof include: alkyl groups, such as a methyl group, an ethyl group, a propyl group, an isopropyl group, an n-butyl group, a sec-butyl group, a tert-butyl group, a 2-ethylhexyl group, and an n-octyl; and saturated cyclic hydrocarbyl groups, such as a cyclopentyl group, a cyclohexyl group, a 1-adamantyl group, and a 2-adamantyl group. Examples of the aryl group include a phenyl group and a naphthyl group. The aryl groups may have a hydroxy group, a halogen atom, or the like.

In the general formula (L6), R^{L20} represents a saturated hydrocarbyl group having 1 to 10 carbon atoms and optionally having a heteroatom, or an aryl group having 6 to 20 carbon atoms and optionally having a heteroatom. Specific examples of the saturated hydrocarbyl group and the aryl group include the same as those exemplified as the saturated hydrocarbyl group and the aryl group, which are represented by R^{L05}, respectively.

In the general formula (L7), R^{L21} represents a saturated hydrocarbyl group having 1 to 10 carbon atoms and optionally having a heteroatom, or an aryl group having 6 to 20 carbon atoms and optionally having a heteroatom. Specific examples of the saturated hydrocarbyl group and the aryl group include the same as those exemplified as the saturated hydrocarbyl group and the aryl group, which are represented by R^{L05}, respectively.

R^{L22} and R^{L23} each independently represent a hydrogen atom or a hydrocarbyl group having 1 to 10 carbon atoms. The hydrocarbyl group may be saturated or unsaturated and may be linear, branched, or cyclic. Specific examples thereof include the same as those exemplified as the hydrocarbyl groups represented by R^{L07} to R^{L16}. R^{L22} and R^{L23} may be bonded to each other to form a substituted or unsubstituted cyclopentane ring or a substituted or unsubstituted cyclohexane ring together with the carbon atoms to which they are bonded. R^{L24} represents a divalent group that forms a substituted or unsubstituted cyclopentane ring, a substituted or unsubstituted cyclohexane ring, or a substituted or unsubstituted norbornane ring together with the carbon atoms to which R^{L24} is bonded. “p” represents 1 or 2.

In the general formula (L8), R^{L25} represents a saturated hydrocarbyl group having 1 to 10 carbon atoms and optionally having a heteroatom, or an aryl group having 6 to 20 carbon atoms and optionally having a heteroatom. Specific examples of the saturated hydrocarbyl group and the aryl group include the same as those exemplified as the saturated hydrocarbyl group and the aryl group, which are represented by R^{L05}, respectively. R^{L26} and R^{L27} represent each independently a hydrogen atom or a hydrocarbyl group having 1 to 10 carbon atoms. The hydrocarbyl group may be saturated or unsaturated and may be linear, branched, or cyclic. Specific examples include the same as those exemplified as the hydrocarbyl groups represented by R^{L07} to R^{L16}. Furthermore, R^{L26} and R^{L27} may be bonded to each other to form a substituted or unsubstituted cyclopentane ring or a substituted or unsubstituted cyclohexane ring together with the carbon atoms to which they are bonded. R^{L28} represents a divalent group that forms a substituted or unsubstituted cyclopentane ring, a substituted or unsubstituted cyclohexane ring, or a substituted or unsubstituted norbornane ring together with the carbon atoms to which R^{L28} is bonded. “q” represents 1 or 2.

In the general formula (L9), R^{L29} represents a saturated hydrocarbyl group having 1 to 10 carbon atoms and optionally having a heteroatom, or an aryl group having 6 to 20 carbon atoms and optionally having a heteroatom. Specific examples of the saturated hydrocarbyl group and aryl group include the same as those exemplified as the saturated hydrocarbyl group and the aryl group, which are represented by R^{L05}, respectively.

R^{L30} and R^{L31} represent each independently a hydrogen atom or a hydrocarbyl group having 1 to 10 carbon atoms. The hydrocarbyl group may be saturated or unsaturated and may be linear, branched, or cyclic. Specific examples thereof include the same as those exemplified as the hydrocarbyl groups represented by R^{L07} to R^{L16}. R^{L30} and R^{L31} may be bonded to each other to form a substituted or unsubstituted cyclopentane ring or a substituted or unsubstituted cyclohexane ring together with the carbon atoms to which they are bonded. R^{L32} represents a divalent group that forms a substituted or unsubstituted cyclopentane ring, a substituted or unsubstituted cyclohexane ring, or a substituted or unsubstituted norbornane ring together with the carbon atom to which R^{L32} is bonded.

Among the acid-labile groups represented by the general formula (L1), specific examples of the linear or branched ones include, but are not limited to, the following.

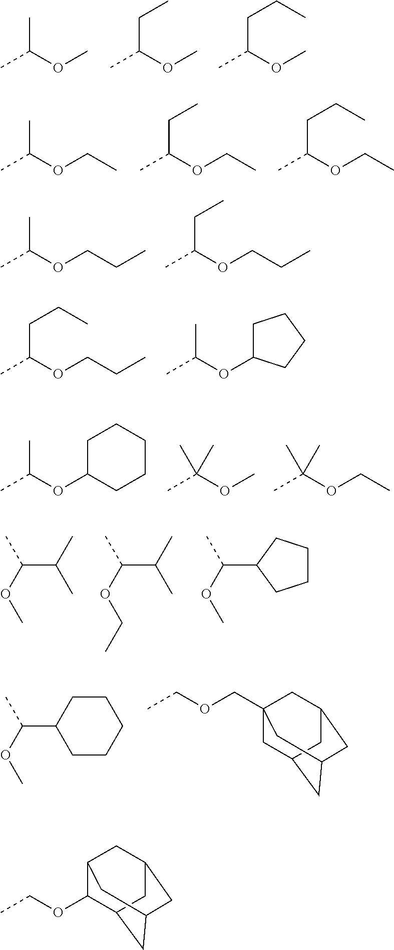

Among the acid-labile groups represented by the general formula (L1), specific examples of cyclic ones include a tetrahydrofuran-2-yl group, a 2-methyltetrahydrofuran-2-yl group, a tetrahydropyran-2-yl group, and a 2-methyltetrahydropyran-2-yl group.

Specific examples of the acid-labile groups represented by the general formula (L2) include a tert-butoxycarbonyl group, a tert-butoxycarbonylmethyl group, a tert-pentyloxycarbonyl group, a tert-pentyloxycarbonylmethyl group, a 1,1-diethylpropyloxycarbonyl group, a 1,1-diethylpropyloxycarbonylmethyl group, a 1-ethylcyclopentyloxycarbonyl group, a 1-ethylcyclopentyloxycarbonylmethyl group, a 1-ethyl-2-cyclopentenyloxycarbonyl group, a 1-ethyl-2-cyclopentenyloxycarbonylmethyl group, a 1-ethoxyethoxycarbonylmethyl group, a 2-tetrahydropyranyloxycarbonylmethyl group, and a 2-tetrahydrofuranyloxycarbonylmethyl group.

Specific examples of the acid-labile group represented by the general formula (L3) include a 1-methylcyclopentyl group, a 1-ethylcyclopentyl group, a 1-n-propylcyclopentyl group, a 1-isopropylcyclopentyl group, a 1-n-butylcyclopentyl group, a 1-sec-butylcyclopentyl group, a 1-tert-butylcyclopentyl group, a 1-cyclohexylcyclopentyl group, a 1-(4-methoxy-n-butyl)cyclopentyl group, a 1-methylcyclohexyl group, a 1-ethylcyclohexyl group, a 3-methyl-1-cyclopenten-3-yl group, a 3-ethyl-1-cyclopenten-3-yl group, a 3-methyl-1-cyclohexen-3-yl group, and a 3-ethyl-1-cyclohexen-3-yl group.

As the acid-labile group represented by the general formula (L4), groups represented by the following formulae (L4-1) to (L4-4) are particularly preferable.

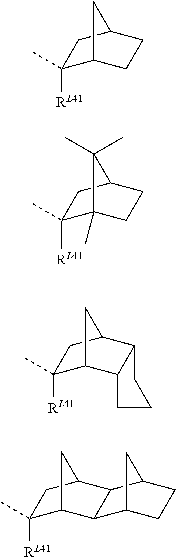

In the formulae, the broken line represents an attachment point and a bond direction. R^{L41} represents as defined above.

In the general formulae (L4-1) to (L4-4), the broken line represents an attachment point and a bond direction. R^{L41} each independently represent a hydrocarbyl group having 1 to 10 carbon atoms. The hydrocarbyl group may be saturated or unsaturated, and may be linear, branched, or cyclic, but a saturated hydrocarbyl group is preferable. Specific examples of the hydrocarbyl group include: alkyl groups, such as a methyl group, an ethyl group, a propyl group, an isopropyl group, an n-butyl group, a sec-butyl group, a tert-butyl group, an n-pentyl group, a tert-pentyl group, and an n-hexyl group; and saturated cyclic hydrocarbyl groups, such as a cyclopentyl group and a cyclohexyl group.

In the general formulae (L4-1) to (L4-4), stereoisomers (enantiomers or diastereomers) may exist, and the general formulae (L4-1) to (L4-4) represent all of these stereoisomers. When the acid-labile group is a group represented by the general formula (L4), multiple stereoisomers may be included.

For example, the general formula (L4-3) represents one or a mixture of two selected from groups represented by the following general formulae (L4-3-1) and (L4-3-2).

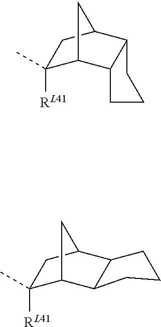

In the formulae, the broken line represents an attachment point and a bond direction. R^{L41} represents as defined above.

Furthermore, the general formula (L4-4) represents one or a mixture of two or more selected from groups represented by the following general formulae (L4-4-1) to (L4-4-4).

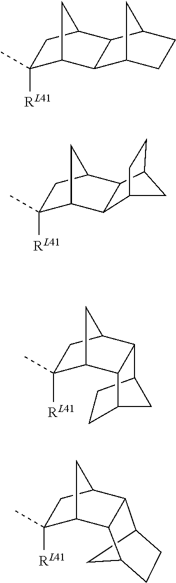

In the formulae, the broken line represents an attachment point and a bond direction. R^{L41} represents the same as defined above.

The general formulae (L4-1) to (L4-4), (L4-3-1), (L4-3-2) and (L4-4-1) to (L4-4-4) represent also enantiomer and mixtures of their enantiomers.

In addition, when the bonding directions of general formulae (L4-1) to (L4-4), (L4-3-1), (L4-3-2), and (L4-4-1) to (L4-4-4) are each at the exo-side relative to the bicyclo[2.2.1]heptane ring, high reactivity in an acid-catalyzed elimination reaction is achieved (see JP2000-336121A). In the production of a monomer having a tertiary exo-saturated hydrocarbyl group having a bicyclo[2.2.1]heptane skeleton as a substituent, there are cases where a monomer substituted with an endo-saturated hydrocarbyl group represented by the following general formulae (L4-1-endo) to (L4-4-endo) is included. In order to achieve favorable reactivity, the exo ratio is preferably 50 mol % or more, and more preferably 80 mol % or more.

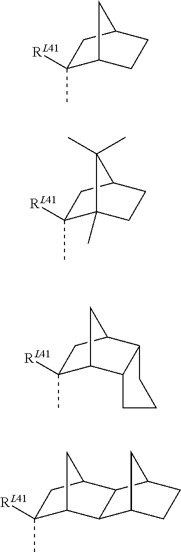

In the formulae, the broken line represents an attachment point and a bond direction. R^{L41} represents the same as defined above.

Examples of the acid-labile group represented by the general formula (L4) include, but are not limited to, the groups shown below.

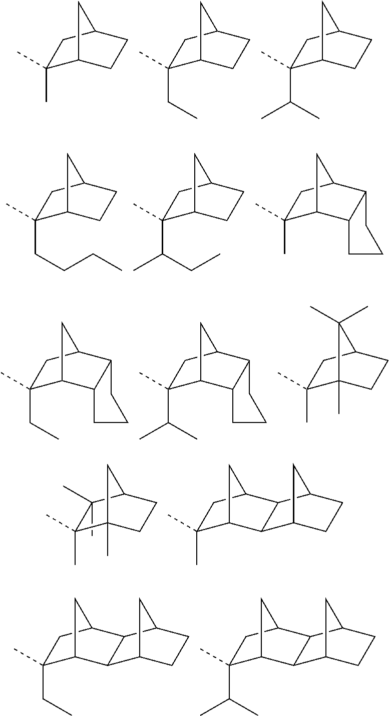

In the formulae, the broken line represents an attachment point and a bond direction.

Examples of the acid-labile group represented by the general formula (L5) include, but are not limited to, a tert-butyl group, a tert-pentyl group, and the groups shown below.

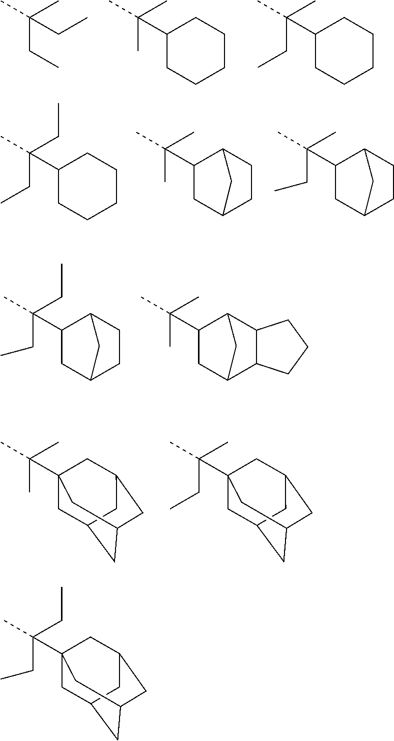

In the formulae, the broken line represents an attachment point.

Examples of the acid-labile group represented by the general formula (L6) include, but are not limited to, the groups shown below.

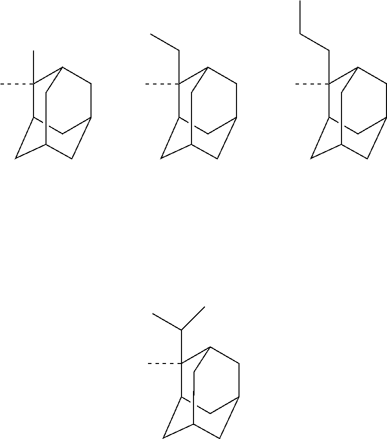

In the formulae, the broken line represents an attachment point.

Examples of the acid-labile group represented by the general formula (L7) include, but are not limited to, the groups shown below.

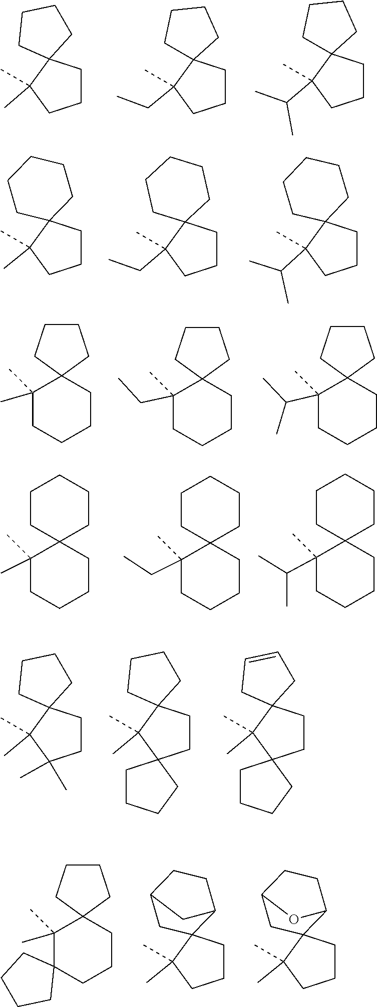

In the formulae, the broken line represents an attachment point.

Examples of the acid-labile group represented by the general formula (L8) include, but are not limited to, the groups shown below.

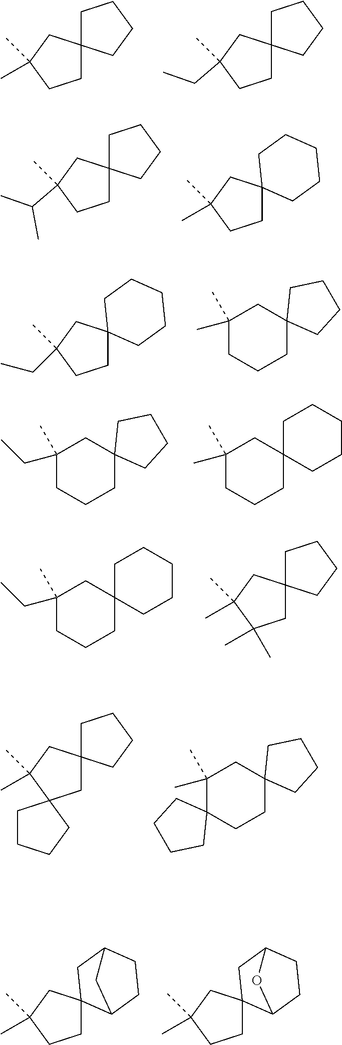

In the formulae, the broken line represents an attachment point.

Examples of the acid-labile group represented by the general formula (L9) include, but are not limited to, the groups shown below.

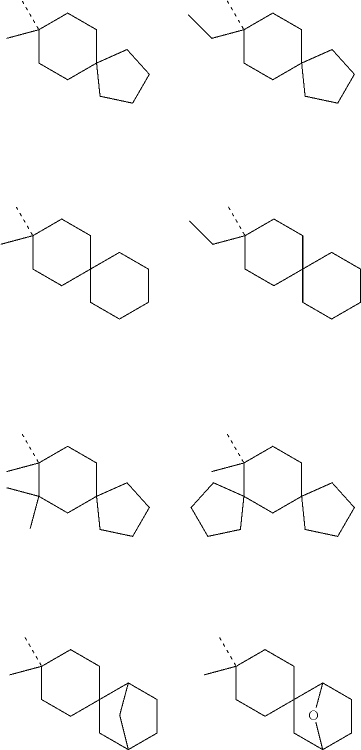

In the formulae, the broken line represents an attachment point.

Specific examples of tertiary hydrocarbyl groups having 4 to 20 carbon atoms, trihydrocarbylsilyl groups in which each hydrocarbyl group has 1 to 6 carbon atoms, and hydrocarbyl groups having 4 to 20 carbon atoms having a carbonyl group, an ether bond, or an ester bond, which are the acid-labile groups represented by R^{AL}, include the same as those exemplified as tertiary hydrocarbyl groups, trihydrocarbylsilyl groups, and saturated hydrocarbyl groups having 4 to 20 carbon atoms and having a carbonyl group, an ether bond, or an ester bond, which are represented by R^{L04}.

Specific examples of the repeating unit represented by the general formula (B1) include, but are not limited to, those shown below. In the following formulae, R^{A} represents the same as defined above.

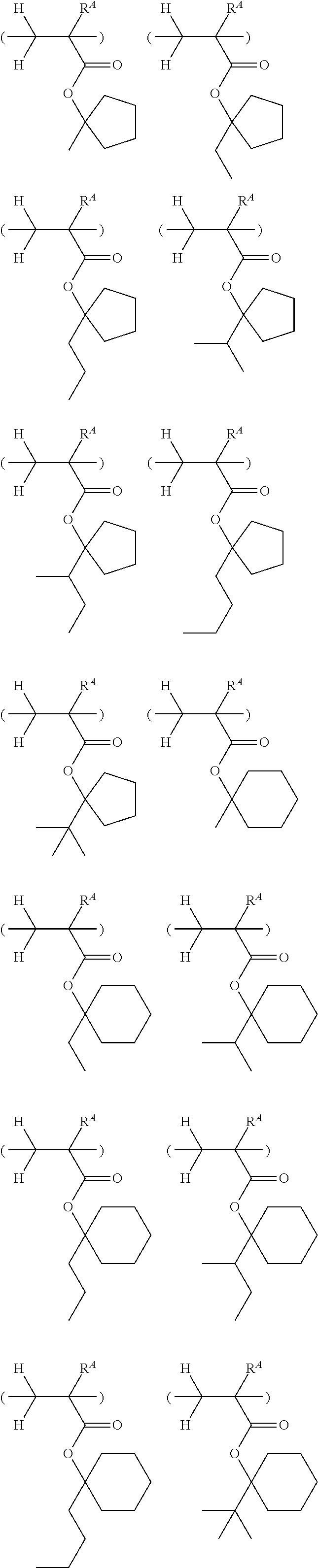

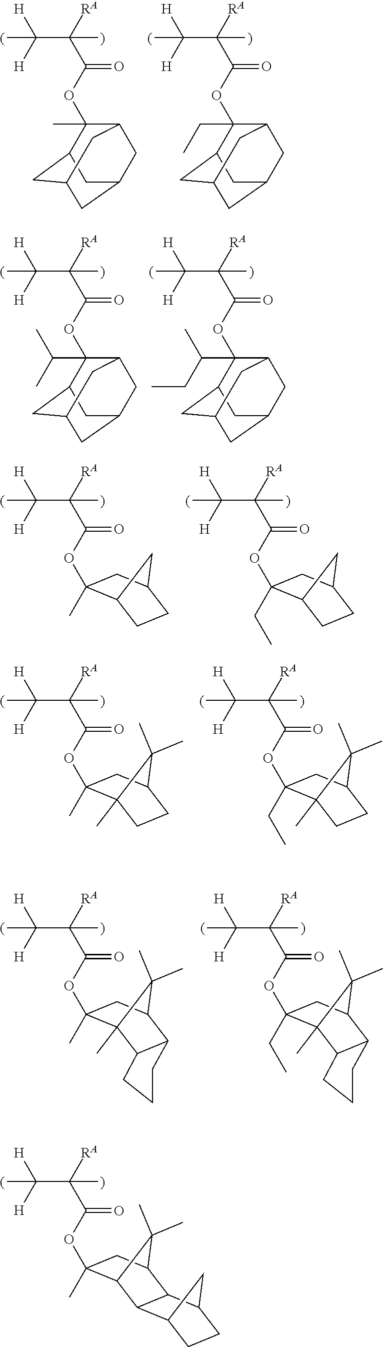

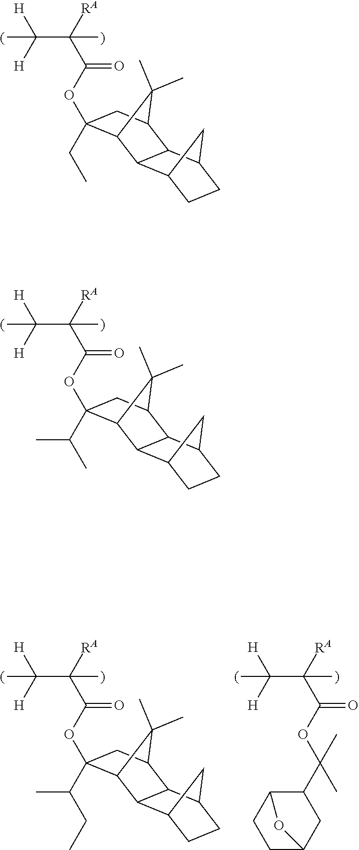

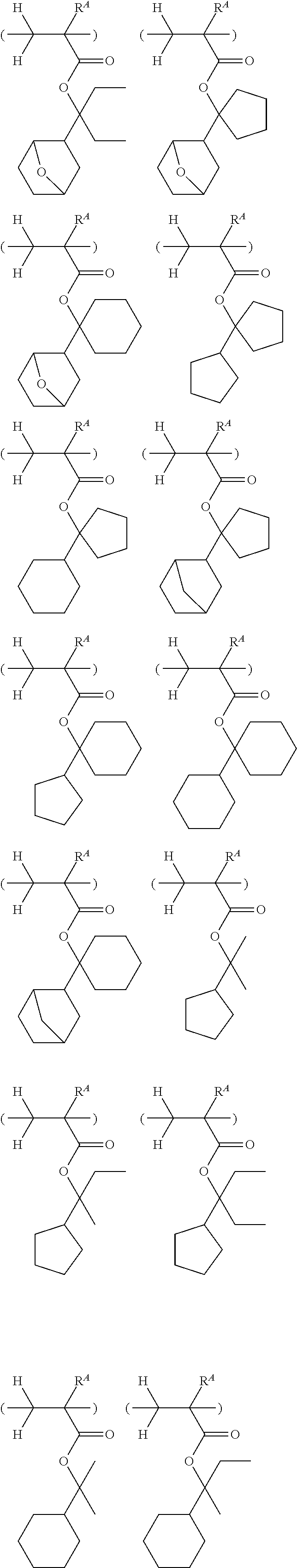

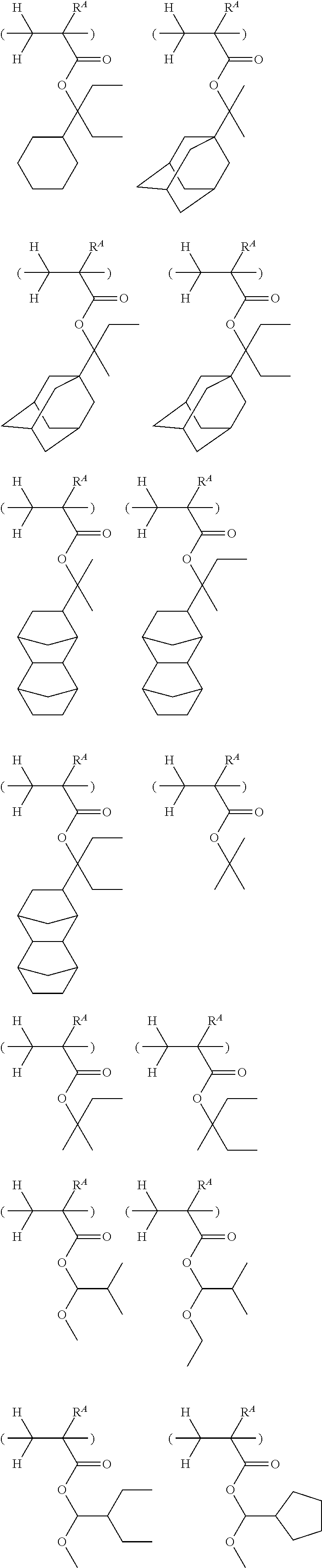

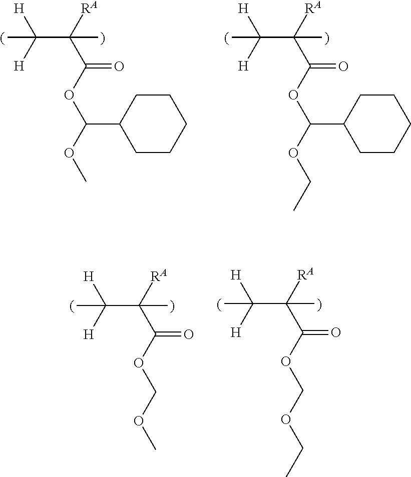

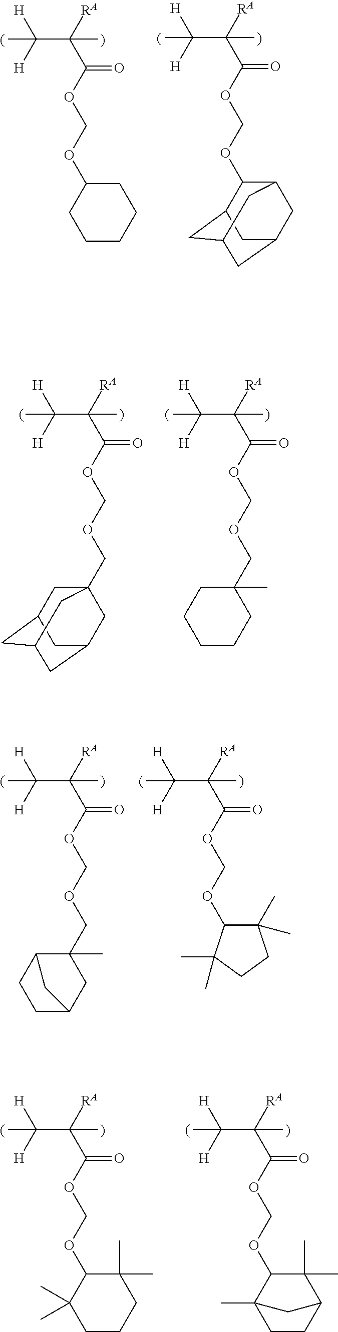

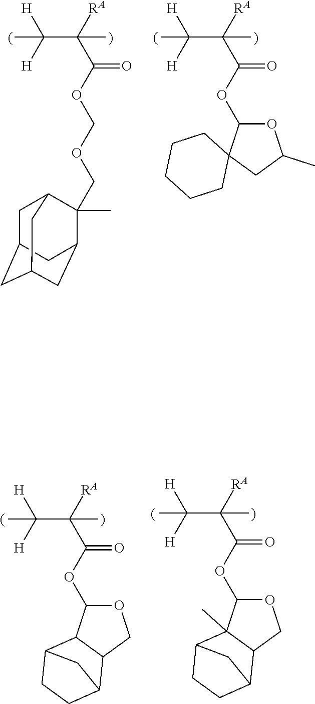

The base polymer may further have at least one repeating unit selected from the group consisting of a repeating unit represented by the following general formula (B2), a repeating unit represented by the following general formula (B3), and a repeating unit represented by the following general formula (B4), as necessary.

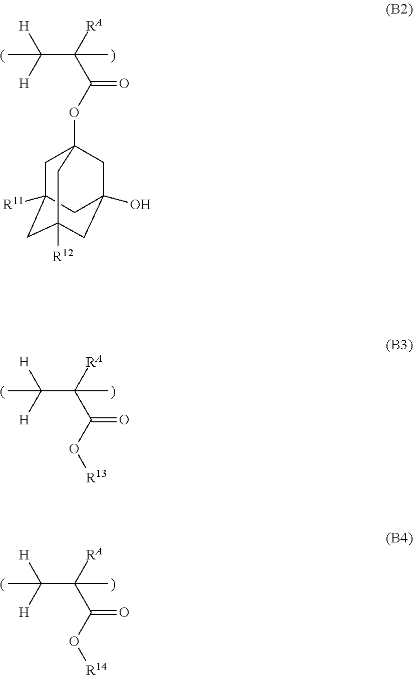

In the formulae,
R^{A} represents a hydrogen atom or a methyl group;
R¹¹ and R¹² each independently represent a hydrogen atom or a hydroxy group;
R¹³ represents a substituent having a lactone structure or a substituent having a sultone structure; and
R¹⁴ represents a hydrogen atom, a fluorinated hydrocarbyl group having 1 to 15 carbon atoms, or a monovalent fluoroalcohol-containing substituent having 1 to 15 carbon atoms.

In the general formulae (B2) to (B4), R^{A} represents a hydrogen atom or a methyl group; R¹¹ and R¹² each independently represent a hydrogen atom or a hydroxy group; R¹³ represents a substituent having a lactone structure or a substituent having a sultone structure; and R¹⁴ represents a hydrogen atom, a fluorinated hydrocarbyl group having 1 to 15 carbon atoms, or a monovalent fluoroalcohol-containing substituent having 1 to 15 carbon atoms.

Specific examples of the repeating unit represented by the general formula (B2) include, but are not limited to, those shown below. In the following formulae, R^{A} represents the same as defined above.

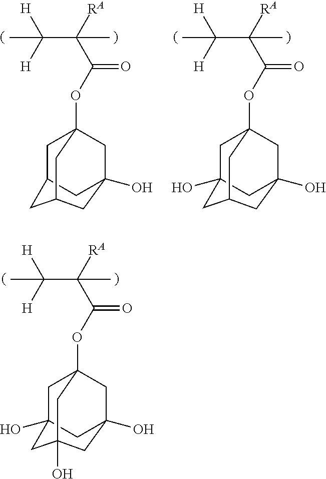

Specific examples of the repeating unit represented by the general formula (B3) include, but are not limited to, those shown below. In the following formulae, R^{A} represents the same as defined above.

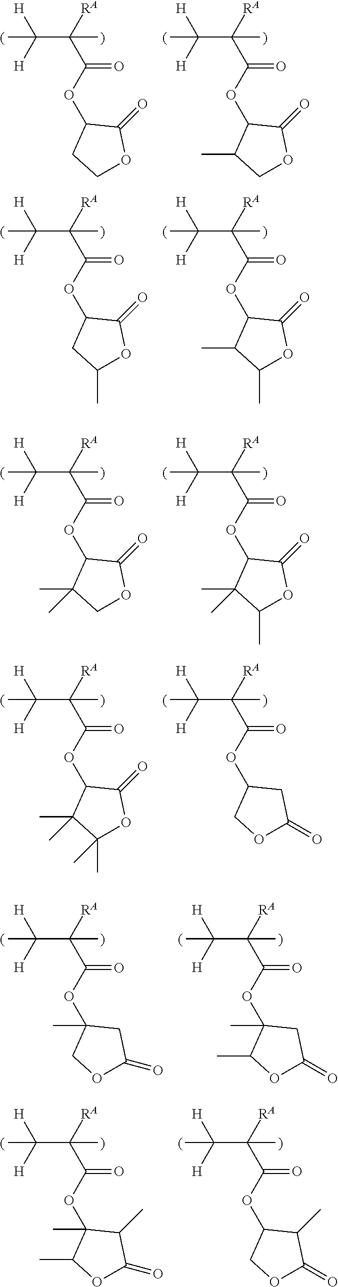

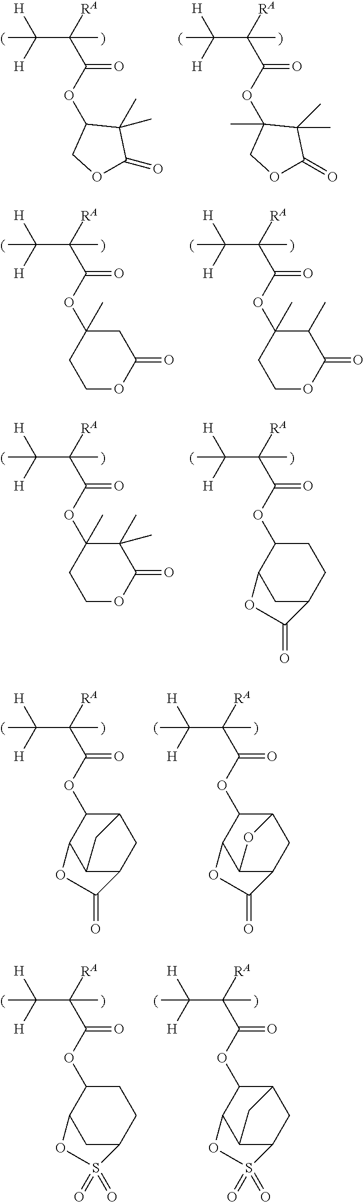

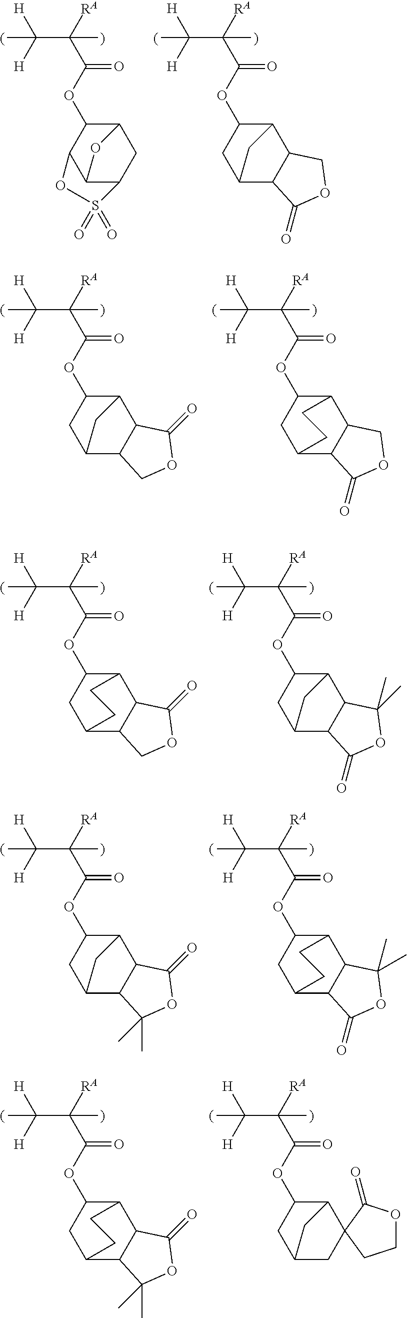

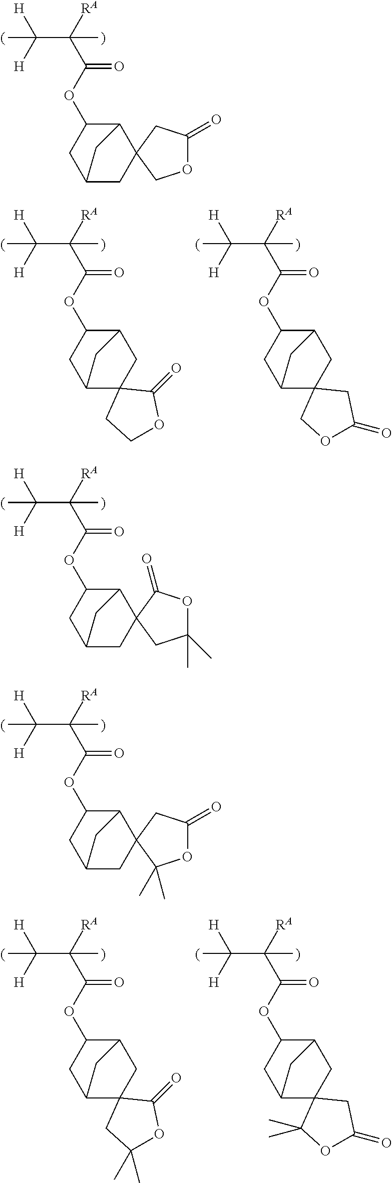

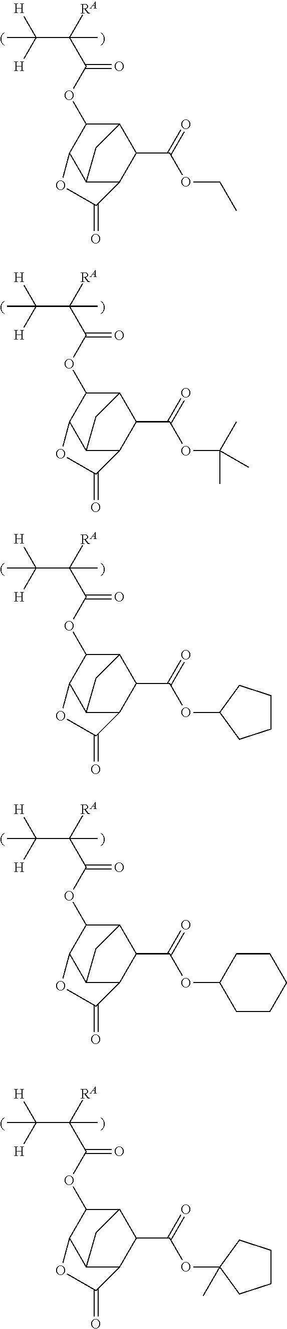

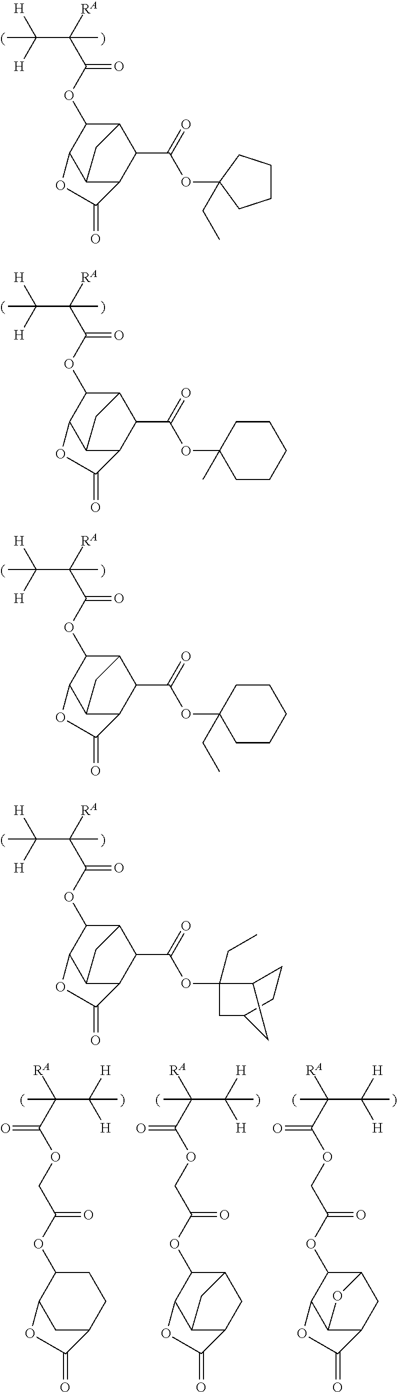

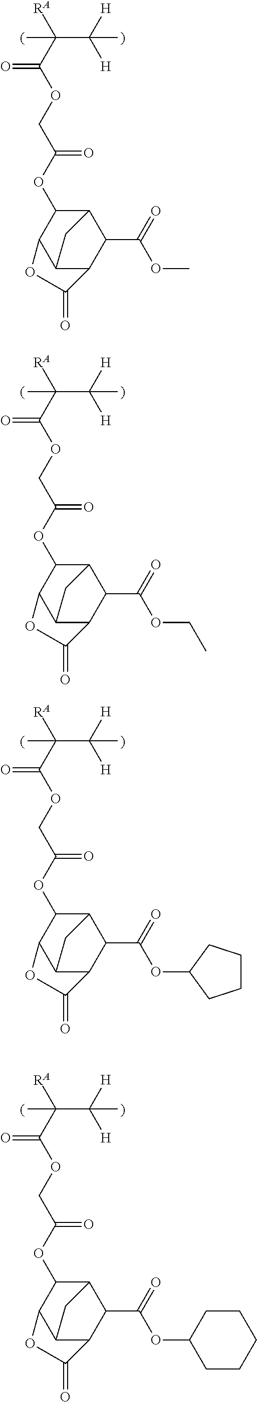

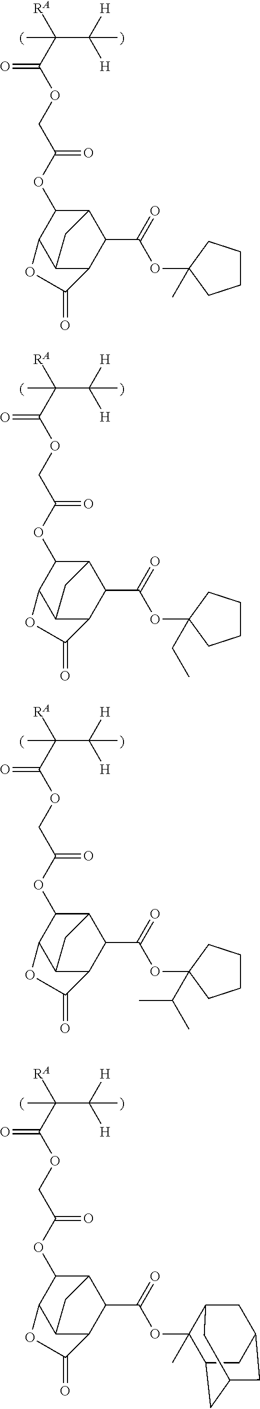

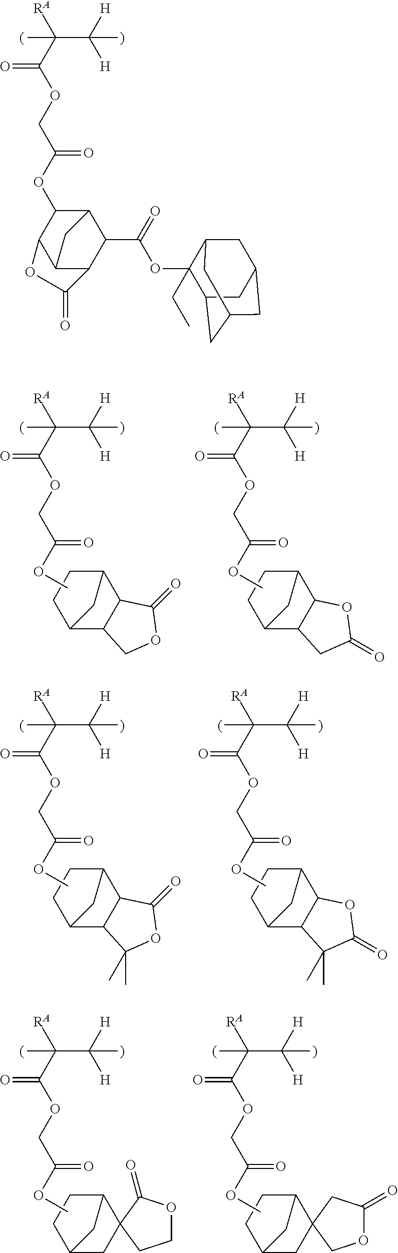

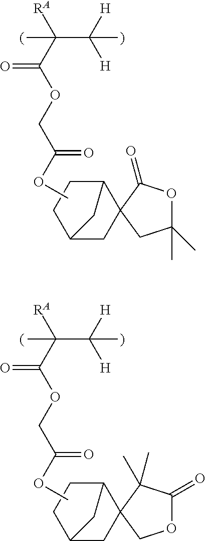

Specific examples of the repeating unit represented by formula (B4) include, but are not limited to, those shown below. In the following formulae, R^{A} represents the same as defined above.

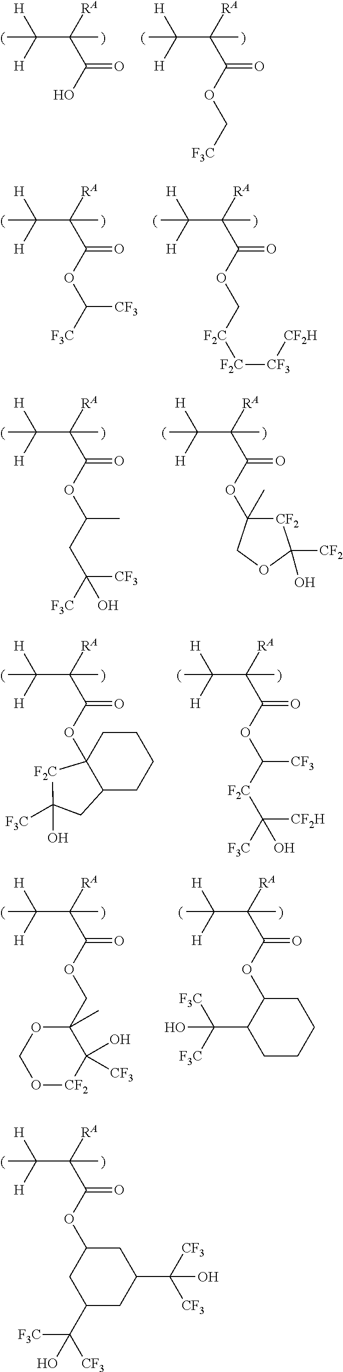

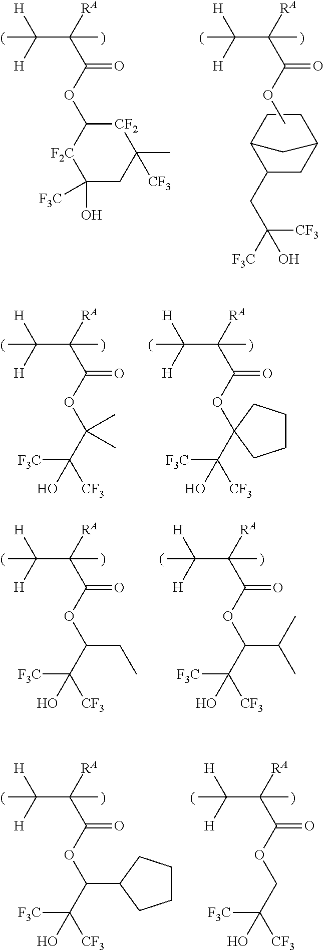

The polymer may further include another repeating unit having no aromatic groups, other than those described above. For example, the polymer may include a repeating unit derived from a monomer having no aromatic groups, such as: substituted acrylic acid esters, such as methyl methacrylate, methyl crotonate, dimethyl maleate, and dimethyl itaconate; unsaturated carboxylic acids, such as maleic acid, fumaric acid, and itaconic acid; cyclic olefins, such as norbornene, norbornene derivatives, and tetracyclo[4.4.0.1^{2,5}.17^{7,10} dodecene derivatives; unsaturated acid anhydrides, such as itaconic acid anhydride; and α-methylene-γ-butyrolactones.

The polymer preferably has a weight-average molecular weight (Mw) of 1000 to 500,000, more preferably 3,000 to 100,000. Within the above range, there is no risk of resolution being degraded because etching resistance is degraded or because it is impossible to ensure contrast between before and after exposure. It should be understood, in the present invention, Mw is a value measured in terms of polystyrene by gel permeation chromatography (GPC) using tetrahydrofuran (THF) as an eluent.

When the polymer has a wide dispersity (Mw/Mn), the low-molecular-weight polymers and high-molecular-weight polymers are present. Thus there is a risk of causing foreign matter on a pattern after the exposure and degrading the pattern profile. The finer the pattern rule, the stronger the influences of such Mw and Mw/Mn. Therefore, in order to obtain a resist composition suitably used for a finer critical dimension, the polymer preferably has a narrow dispersity Mw/Mn of 1.0 to 2.0, particularly preferably 1.0 to 1.5.

Examples of methods for synthesizing the polymer include a method of polymerizing the monomers that give the repeating unit represented by the general formula (B1), and as necessary, the repeating units represented by the general formulae (B2) to (B4) and other repeating units, by heating with the addition of a radical initiator, in an organic solvent. Examples of the organic solvent used in the polymerization reaction include toluene, benzene, THF, diethyl ether, dioxane, methyl ethyl ketone, γ-butyrolactone, and propylene glycol monomethyl ether acetate (PGMEA). Examples of the polymerization initiator include 2,2′-azobisisobutyronitrile (AIBN), 2,2′-azobis(2,4-dimethylvaleronitrile), dimethyl-2,2-azobis(2-methylpropionate), benzoyl peroxide, and lauroyl peroxide. The adding amount of these initiators is preferably 0.01 to 25 mol % relative to the total of the monomers to be polymerized. The reaction temperature of the polymerization reaction is preferably 50 to 150 °C, more preferably 60 to 100 °C. The reaction time is preferably 2 to 24 hours. An acid-labile group introduced into the monomer may be used as it is, or may be protected or partially protected after polymerization. Furthermore, to adjust the molecular weight, polymerization may be performed using a publicly known chain transfer agent, such as dodecyl mercaptan and 2-mercaptoethanol. In this case, the adding amount of the chain transfer agent is preferably an amount to be 0.01 to 10 in mole ratio relative to the total of the monomers to be polymerized.

In the polymer, preferable content ratios of the repeating units can be each, for example, in the ranges shown below, but are not limited thereto.
(I) Preferably, 1 to 99 mol %, more preferably 20 to 95 mol %, further preferably 30 to 90 mol %, of the repeating unit represented by the general formula (B1) can be contained, and as necessary,
(II) preferably, 0 to 99 mol %, more preferably 1 to 90 mol %, further preferably 10 to 70 mol %, of at least one selected from the repeating unit represented by the general formula (B2), the repeating unit represented by the general formula (B3), and the repeating unit represented by the general formula (B4) can be contained, and
(III) preferably, 0 to 99 mol %, more preferably 0 to 70 mol %, and further preferably 0 to 50 mol % of other repeating units can be contained.

One kind of the component (B) base polymer may be used, or two or more kinds thereof, having different composition ratios, Mw, and/or Mw/Mn, may be used in combination.

### [(C) Organic Solvent]

The inventive chemically-amplified resist composition may contain an organic solvent as the component (C). Examples of the organic solvent include ones disclosed in paragraphs [0144] and [0145] of JP2008-111103A: ketones, such as cyclohexanone, and methyl-2-n-pentyl ketone; alcohols, such as 3-methoxybutanol, 3-methyl-3-methoxybutanol, 1-methoxy-2-propanol, 1-ethoxy-2-propanol, and diacetone alcohol; ethers, such as propylene glycol monomethyl ether, ethylene glycol monomethyl ether, propylene glycol monoethyl ether, ethylene glycol monoethyl ether, propylene glycol dimethyl ether, and diethylene glycol dimethyl ether; esters, such as PGMEA, propylene glycol monoethyl ether acetate, ethyl lactate, ethyl pyruvate, butyl acetate, methyl 3-methoxypropionate, ethyl 3-ethoxypropionate, tert-butyl acetate, tert-butyl propionate, and propylene glycol mono-tert-butyl ether acetate; lactones, such as γ-butyrolactone; and mixed solvents thereof. When an acetal-based acid-labile group is used, a high-boiling alcohol solvent, specifically, diethylene glycol, propylene glycol, glycerin, 1,4-butanediol, 1,3-butanediol, etc., can be added in order to accelerate the deprotection reaction of the acetal.

In the inventive chemically-amplified resist composition, the contained amount of the component (C) organic solvent is preferably 100 to 10,000 parts by mass, more preferably 300 to 8,000 parts by mass relative to 80 parts by mass of the component (B) base polymer. One kind of the organic solvent (C) may be used, or two or more kinds thereof may be used in mixture.

### [(D) Fluorine-Atom-Containing Polymer]

The inventive chemically-amplified resist composition may further contain a polymer, which is different from the component (B) base polymer and is a fluorine-atom-containing polymer having at least one selected from the group consisting of a repeating unit represented by the following general formula (D1), a repeating unit represented by the following general formula (D2), a repeating unit represented by the following general formula (D3), and a repeating unit represented by the following general formula (D4).

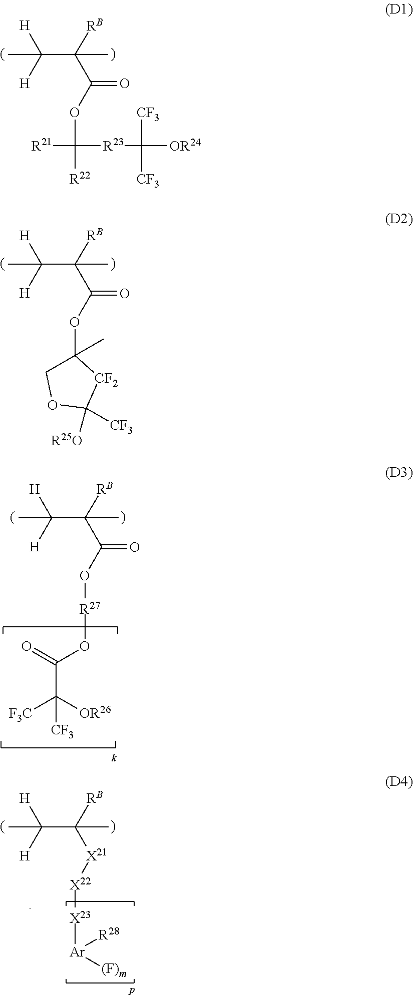

In the general formulae (D1) to (D4), R^{B} represents a hydrogen atom or a methyl group. R²¹ and R²² each independently represent a hydrogen atom or a hydrocarbyl group having 1 to 10 carbon atoms. R²³ represents a single bond or a linear or branched hydrocarbylene group having 1 to 5 carbon atoms. R²⁴, R²⁵, and R²⁶ each independently represent a hydrogen atom, a hydrocarbyl group having 1 to 15 carbon atoms, a fluorinated hydrocarbyl group having 1 to 15 carbon atoms, an acyl group having 2 to 15 carbon atoms, or an acid-labile group. When R²⁴, R²⁵, and R²⁶ are a hydrocarbyl group or a fluorinated hydrocarbyl group, part of their —CH₂— may be substituted with an ether bond or a carbonyl group. R²⁷ represents a (k+1)-valent hydrocarbon group having 1 to 20 carbon atoms or a (k+1)-valent fluorinated hydrocarbon group having 1 to 20 carbon atoms. “k” represents an integer of 1 to 3. X²¹ represents each independently a single bond, an ester bond, an ether bond, an amide bond, or a phenylene bond. “p” represents an integer of 1 or 2. X²² is each independently a single bond or a hydrocarbylene group having 1 to 20 carbon atoms when “p” is 1, or X²² is a trivalent hydrocarbon group having 1 to 20 carbon atoms when “p” is 2, and the hydrocarbylene group and the trivalent hydrocarbon group may have at least one atom selected from an oxygen atom, a sulfur atom, a nitrogen atom, and a halogen atom. X²³ each independently represents a single bond, an ester bond, an ether bond, an amide bond, a sulfonate ester bond, a urethane bond, a thiourethane bond, or a urea bond. Ar represents a group derived from benzene or naphthalene. R²⁸ each independently represents a hydrogen atom, a saturated hydrocarbyl group having 1 to 6 carbon atoms, a saturated hydrocarbyloxy group having 1 to 6 carbon atoms, a saturated hydrocarbyloxycarbonyl group having 2 to 6 carbon atoms, a hydroxy group, a carboxy group, a halogen atom, a cyano group, or a nitro group. “m” represents an integer of 1 to 5.

The hydrocarbyl groups having 1 to 10 carbon atoms and represented by R²¹ and R²² are preferably saturated hydrocarbyl groups, and may be linear, branched, or cyclic. Specific examples include alkyl groups having 1 to 10 carbon atoms, such as a methyl group, an ethyl group, an n-propyl group, an isopropyl group, a cyclopropyl group, an n-butyl group, an isobutyl group, a sec-butyl group, a tert-butyl group, a cyclobutyl group, an n-pentyl group, an n-hexyl group, an n-heptyl, an n-octyl group, an n-nonyl group, and an n-decyl group; and saturated cyclic hydrocarbyl groups having 3 to 10 carbon atoms, such as a cyclopentyl group, a cyclohexyl group, an adamantyl group, and a norbornyl group. Among these, those having 1 to 6 carbon atoms are preferable.

The hydrocarbylene group having 1 to 5 carbon atoms and represented by R²³ is preferably a saturated hydrocarbylene group, which may be linear, branched, or cyclic. Specific examples thereof include a methylene group, an ethylene group, a trimethylene group, a propylene group, a tetramethylene group, and a pentamethylene group.

The hydrocarbyl groups having 1 to 15 carbon atoms and represented by R²⁴, R²⁵, and R²⁶ may be saturated or unsaturated, and may be linear, branched, or cyclic. Specific examples thereof include unsaturated aliphatic hydrocarbyl groups, such as saturated hydrocarbyl groups, alkenyl groups, and alkynyl groups, but saturated hydrocarbyl groups are preferable. Examples of the saturated hydrocarbyl groups include those exemplified as specific examples of the hydrocarbyl groups having 1 to 10 carbon atoms and represented by R²¹ and R²², as well as an n-undecyl group, an n-dodecyl group, a tridecyl group, a tetradecyl group, and a pentadecyl group. Examples of the fluorinated hydrocarbyl groups having 1 to 15 carbon atoms include the aforementioned hydrocarbyl groups in which part or all of the hydrogen atoms bonded to a carbon atom are substituted with a fluorine atom. Furthermore, part of the —CH₂— of the hydrocarbyl groups and fluorinated hydrocarbyl groups may be substituted with an ether bond or a carbonyl group.

When R²⁴, R²⁵, and R²⁶ are acid-labile groups, specific examples thereof include: the groups represented by the above-mentioned general formulae (L1) to (L9); tertiary hydrocarbyl groups having 4 to 20, preferably 4 to 15, carbon atoms; trihydrocarbylsilyl groups in which each hydrocarbyl group has 1 to 6 carbon atoms; and hydrocarbyl groups having 4 to 20 carbon atoms and having a carbonyl group, an ether bond, or an ester bond.

The (k+1)-valent hydrocarbon or fluorinated hydrocarbon group, each having 1 to 20 carbon atoms and represented by R²⁷, includes groups in which k hydrogen atoms have been further removed from the aforementioned hydrocarbyl or fluorinated hydrocarbyl groups.

Examples of the repeating unit represented by the general formula (D1) include, but are not limited to, those shown below. In the following formulae, R^{B} represents the same as defined above.

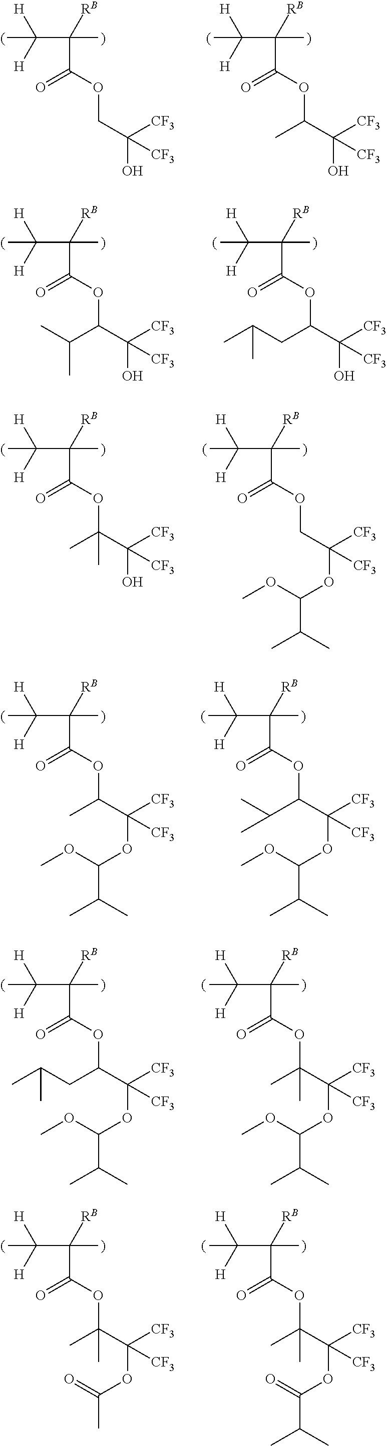

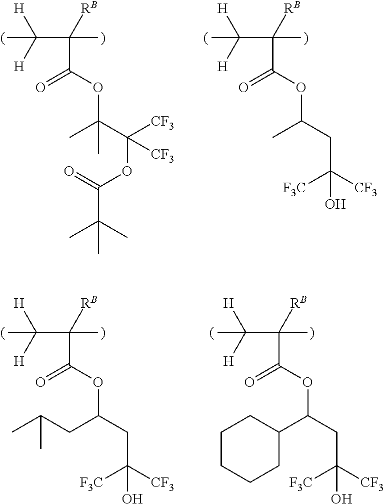

Examples of the repeating unit represented by the general formula (D2) include those shown below, but are not limited thereto. In the following formulae, R^{B} represents the same as defined above.

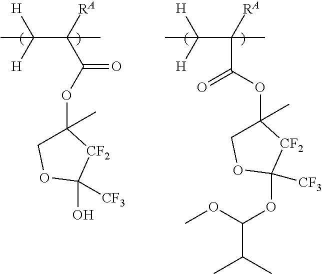

Examples of the repeating unit represented by the general formula (D3) include those shown below, but are not limited thereto. In the following formulae, R^{B} represents the same as defined above.

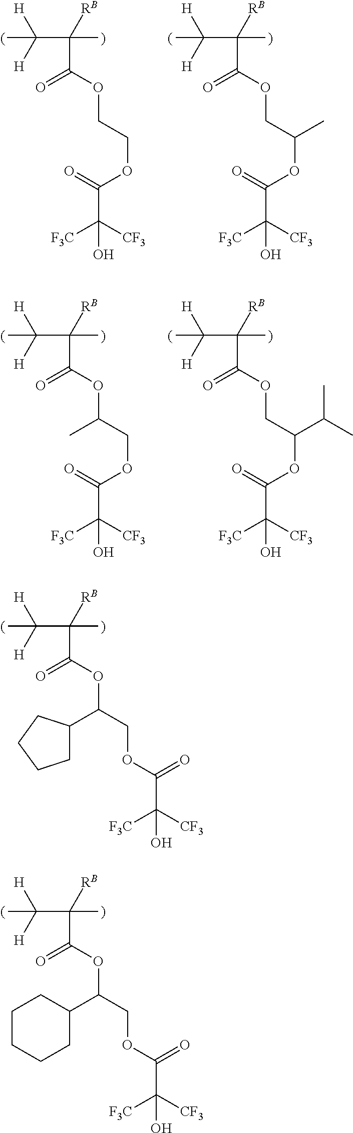

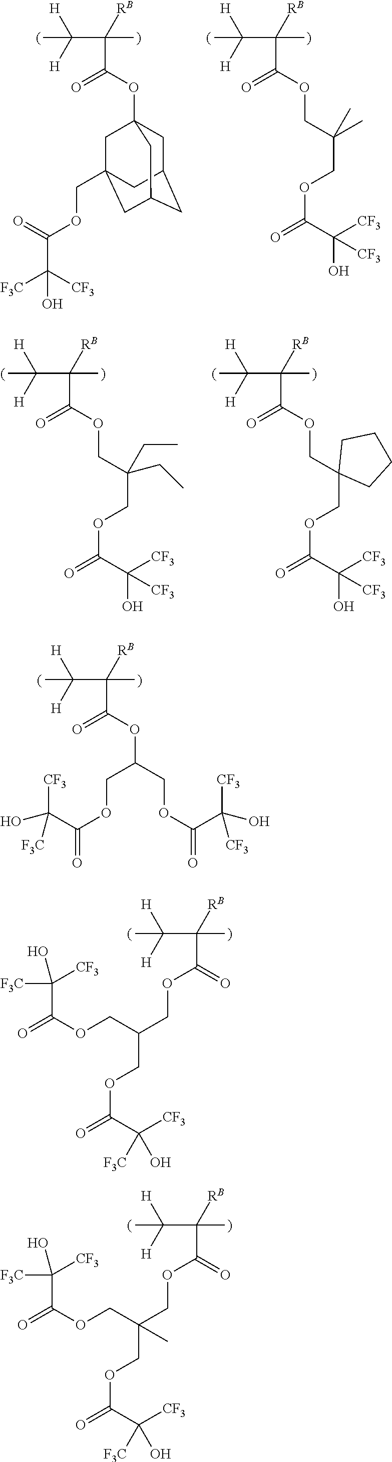

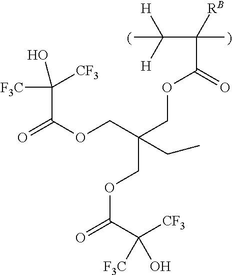

Examples of the repeating unit represented by the general formula (D4) include those shown below, but are not limited thereto. In the following formulae, R^{B} represents the same as defined above.

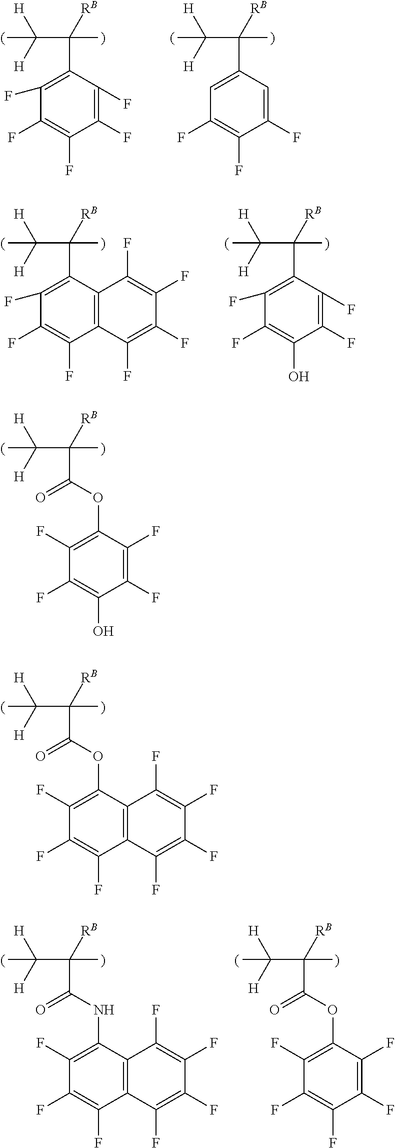

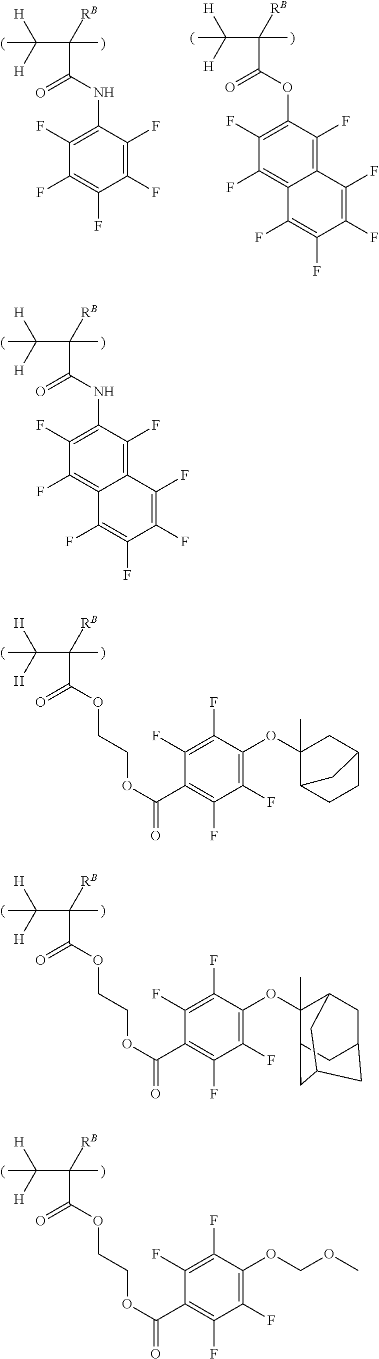

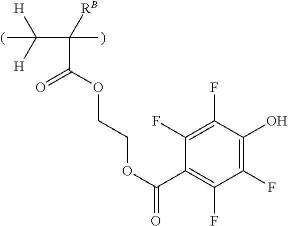

The fluorine-atom-containing polymer may further have other repeating units other than the repeating units represented by any of the general formulae (D1) to (D4). Specific examples of the other repeating units include repeating units obtained from methacrylic acid, α-trifluoromethylacrylic acid derivatives, etc. In the polymer-type surfactant, the contained amount of the repeating units represented by the general formulae (D1) to (D4) is preferably 20 mol % or more, more preferably 60 mol % or more, and further preferably 100 mol % of all the repeating units.

The Mw of the fluorine-atom-containing polymer of the component (D) is preferably 1,000 to 100,000, more preferably 3,000 to 1,5000. Mw/Mn is preferably 1.0 to 2.0, more preferably 1.0 to 1.6.

Examples of methods for synthesizing the fluorine-containing resin of the component (D) include a method of polymerizing the monomers to give at least one selected from the repeating unit represented by the general formula (D1), the repeating unit represented by the general formula (D2), the repeating unit represented by the general formula (D3), and the repeating unit represented by the general formula (D4), and, as necessary, the monomers to give other repeating units, by heating with the addition of a radical initiator, in an organic solvent. Examples of the organic solvent used in the polymerization reaction include toluene, benzene, THF, diethyl ether, dioxane, methyl ethyl ketone, propylene glycol monomethyl ether, and PGMEA. Examples of the polymerization initiator include AIBN, 2,2′-azobis(2,4-dimethylvaleronitrile), dimethyl-2,2-azobis(2-methylpropionate), benzoyl peroxide, and lauroyl peroxide. The reaction temperature of the polymerization reaction is preferably 50 to 100 °C. The adding amount of these initiators is preferably 0.01 to 25 mol % relative to the total of the monomers to be polymerized. The reaction time is preferably 4 to 24 hours. An acid-labile group introduced into the monomer may be used as it is, or may be protected or partially protected after polymerization. Furthermore, to adjust the molecular weight, polymerization may be performed using a publicly known chain transfer agent, such as dodecyl mercaptan or 2-mercaptoethanol. In this case, the adding amount of the chain transfer agent is preferably an amount to be 0.01 to 10 in mole ratio relative to all the monomers to be polymerized.

When the inventive chemically-amplified resist composition contains the component (D) fluorine-atom-containing polymer, the contained amount is preferably 0.1 to 50 parts by mass, more preferably 0.5 to 10 parts by mass relative to 80 parts by mass of the component (B) base polymer. When the contained amount of the fluorine-atom-containing polymer (D) is in the above range, the contact angle between the resist film surface and water is enhanced sufficiently, and thus it is possible to suppress defects due to immersion water remaining and elution of the acid generator or quencher. Furthermore, it is possible to adjust the solubility of the resist film surface, and favorable CDU can be achieved. One kind of the fluorine-atom-containing polymer (D) may be used, or two or more kinds thereof may be used in combination.

### [(E) Quencher]

The inventive chemically-amplified resist composition may further contain a quencher as the component (E), as necessary. In the present invention, a quencher means a compound capable of trapping the acid generated from a photoacid generator to prevent the acid from diffusing to unexposed portions.

Specific examples of the quencher include amine compounds, sulfonate salts, and carboxylate salts.

As the amine compounds, preferable compounds are primary, secondary, or tertiary amine compounds disclosed in paragraphs [0146] to [0164] of JP2008-111103A, especially amine compounds having any of a hydroxy group, an ether bond, an ester bond, a lactone ring, a cyano group, a sulfonic acid ester bond, etc. In addition, a compound in which a primary or secondary amine is protected as a carbamate group, like the compound disclosed in JP3790649B2, is also preferable. Such a protected amine compound is effective when there is, in the resist composition, a component that is unstable to a base.

Specific examples of the sulfonate salts include compounds represented by the following general formula (E1). Specific examples of the carboxylate salts include compounds represented by the following general formula (E2).

R³¹—SO₃⁻Mq⁺ (E1)

R³²—Co₂⁻Mq⁺ (E2)

In the general formula (E1), R³¹ represents a hydrogen atom or a hydrocarbyl group having 1 to 40 carbon atoms and optionally having a heteroatom, but excludes groups in which a hydrogen atom bonded to the carbon atom at the α-position of the sulfo group is substituted with a fluorine atom or a fluoroalkyl group.

The hydrocarbyl group having 1 to 40 carbon atoms and represented by R³¹ may be saturated or unsaturated, and may be linear, branched, or cyclic. Specific examples thereof include: alkyl groups having 1 to 40 carbon atoms, such as a methyl group, an ethyl group, a propyl group, an isopropyl group, an n-butyl group, a sec-butyl group, a tert-butyl group, a tert-pentyl group, an n-pentyl group, an n-hexyl group, an n-octyl group, a 2-ethylhexyl group, an n-nonyl group, and an n-decyl group; saturated cyclic hydrocarbyl groups having 3 to 40 carbon atoms, such as a cyclopentyl group, a cyclohexyl group, a cyclopentylmethyl group, a cyclopentylethyl group, a cyclopentylbutyl group, a cyclohexylmethyl group, a cyclohexylethyl group, a cyclohexylbutyl group, a norbornyl group, a tricyclo[5.2.1.0^{2,6}] decanyl group, an adamantyl group, and an adamantylmethyl group; alkenyl groups having 2 to 40 carbon atoms, such as a vinyl group, a 1-propenyl group, a 2-propenyl group, a butenyl group, and a hexenyl group; unsaturated aliphatic cyclic hydrocarbyl groups having 3 to 40 carbon atoms, such as a cyclohexenyl group; aryl groups having 6 to 40 carbon atoms, such as a phenyl group, a naphthyl group, alkylphenyl groups (a 2-methylphenyl group, a 3-methylphenyl group, a 4-methylphenyl group, a 4-ethylphenyl group, a 4-tert-butylphenyl group, and a 4-n-butylphenyl group), dialkylphenyl groups (a 2,4-dimethylphenyl group and a 2,4,6-triisopropylphenyl group), alkylnaphthyl groups (a methylnaphthyl group and an ethylnaphthyl group), and dialkylnaphthyl groups (a dimethylnaphthyl group and a diethylnaphthyl group); and aralkyl groups having 7 to 40 carbon atoms, such as a benzyl group, a 1-phenylethyl group, and a 2-phenylethyl group.

Moreover, part or all of the hydrogen atoms of the hydrocarbyl groups may be substituted with a group having a heteroatom, such as an oxygen atom, a sulfur atom, a nitrogen atom, or a halogen atom, and part of the —CH₂— of the hydrocarbyl groups may be substituted with a group having a heteroatom, such as an oxygen atom, a sulfur atom, or a nitrogen atom. As a result, the hydrocarbyl groups may have a hydroxy group, a fluorine atom, a chlorine atom, a bromine atom, an iodine atom, a cyano group, a carbonyl group, an ether bond, a thioether bond, an ester bond, a sulfonic acid ester bond, a carbonate bond, a lactone ring, a sultone ring, carboxylic anhydride (—C(═O)—O—C(═O)—), a haloalkyl group, etc. Examples of the hydrocarbyl group having a heteroatom include: heteroaryl groups, such as a thienyl group; alkoxyphenyl groups, such as a 4-hydroxyphenyl group, a 4-methoxyphenyl group, a 3-methoxyphenyl group, a 2-methoxyphenyl group, a 4-ethoxyphenyl group, a 4-tert-butoxyphenyl group, and a 3-tert-butoxyphenyl group; alkoxynaphthyl groups, such as a methoxynaphthyl group, an ethoxynaphthyl group, an n-propoxynaphthyl group, and an n-butoxynaphthyl group; dialkoxynaphthyl groups, such as a dimethoxynaphthyl group and a diethoxynaphthyl group; and aryloxoalkyl groups, such as 2-aryl-2-oxoethyl groups, such as a 2-phenyl-2-oxoethyl group, a 2-(1-naphthyl)-2-oxoethyl group, and a 2-(2-naphthyl)-2-oxoethyl group.

R³² represents a hydrocarbyl group having 1 to 40 carbon atoms and optionally having a heteroatom. Examples of the hydrocarbyl group represented by R³² include the same as those exemplified as the hydrocarbyl group represented by R³¹. In addition, other specific examples thereof include fluorine-containing alkyl groups, such as a trifluoromethyl group, a trifluoroethyl group, a 2,2,2-trifluoro-1-methyl-1-hydroxyethyl group, and a 2,2,2-trifluoro-1-(trifluoromethyl)-1-hydroxyethyl group; and fluorine-containing aryl groups, such as a pentafluorophenyl group and a 4-trifluoromethylphenyl group.

Specific examples of the anion of the sulfonate salt represented by the general formula (E1) include those shown below, but are not limited thereto.

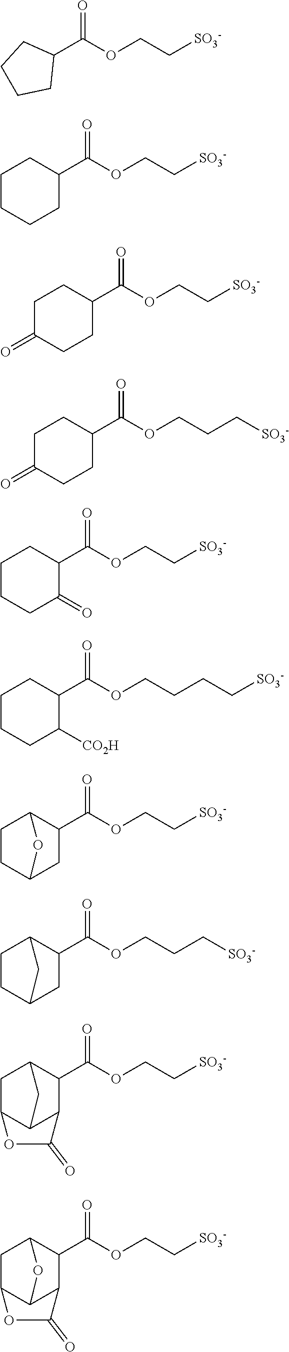

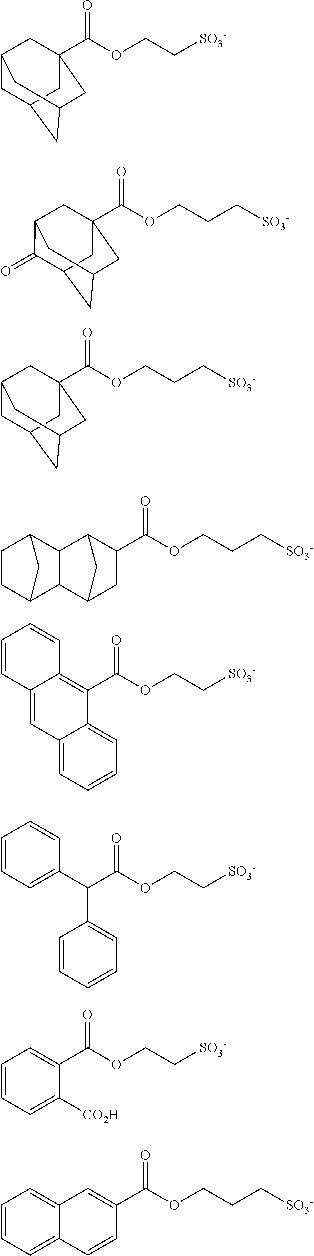

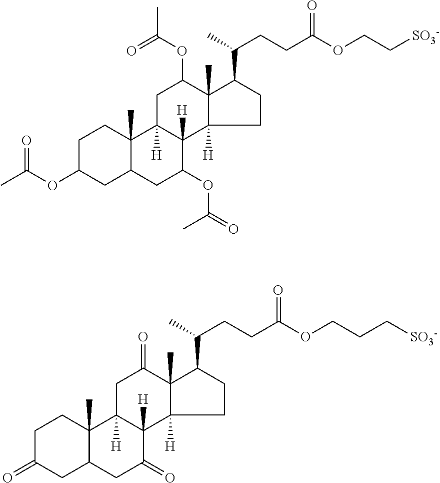

Specific example of the anion of the carboxylate salt represented by the general formula (E2) include those shown below, but are not limited thereto.

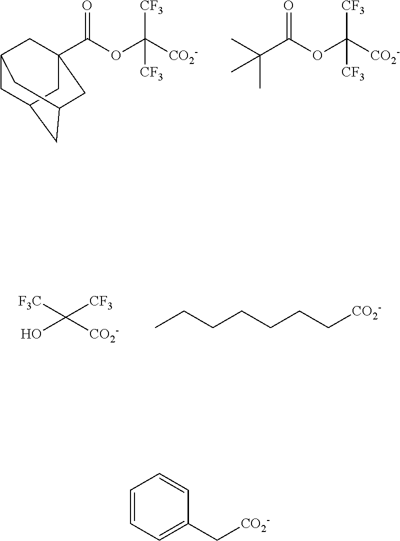

In the general formulae (1) and (2), Mq⁺ represents an onium cation. Examples of the onium cation include a sulfonium cation, an iodonium cation, and an ammonium cation.

The sulfonium cations are preferably those represented by the general formula (Z-1), and specific examples thereof include the same as those exemplified as specific examples of the sulfonium cation represented by the general formula (Z-1). The iodonium cations are preferably those represented by the general formula (Z-2), and specific examples thereof include the same as those exemplified as specific examples of the iodonium cation represented by the general formula (Z-2).

When the inventive chemically-amplified resist composition contains the component (E) quencher, the contained amount is preferably 0.001 to 12 parts by mass, more preferably 0.01 to 8 parts by mass relative to 80 parts by mass of the component (B) base polymer. By blending the component (E) quencher, the sensitivity adjustment of the resist film is facilitated, and in addition, acid diffusion in the resist film is suppressed, and resolution is enhanced. Thus, it is possible to suppress sensitivity changes after exposure and reduce dependence on the substrate and the environment, making it possible to enhance exposure latitude, pattern profile, etc. Furthermore, by adding the quencher, it is also possible to enhance adhesiveness to a substrate. One kind of the quencher may be used, or two or more kinds thereof may be used in combination.

### [(F) Other Photoacid Generator]

Furthermore, the inventive chemically-amplified resist composition may contain a photoacid generator (hereinafter, also referred to as “other photoacid generators”) other than the component (A) photoacid generator in order to fine-tune lithography performance. The other photoacid generator may be any compound as long as it generates an acid upon exposure to high-energy radiation, and may be publicly known compounds used in conventional resist compositions, particularly chemically-amplified resist compositions. Suitable other photoacid generators include sulfonium salts, iodonium salts, sulfonyldiazomethane, N-sulfonyloxyimides, and oxime-O-sulfonate-type acid generators, one of which may be used or two or more kinds of which may be used in combination. The acid generated from the other photoacid generators is preferably a strong acid, such as sulfonic acid, (bisperfluoroalkanesulfonyl)imide, or (trisperfluoromethanesulfonyl)methide, or a weak acid, such as carboxylic acid.

Specific examples of the other photoacid generators include onium salts represented by the following general formula (F1) or (F2).

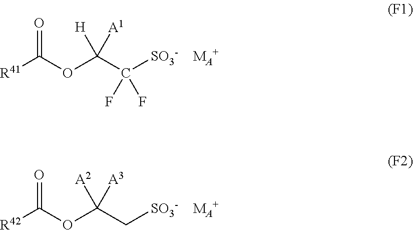

In the above general formulae (F1) and (F2), A1 represents a hydrogen atom or a trifluoromethyl group. A2 and A3 each independently represent a hydrogen atom or a trifluoromethyl group, but are not both hydrogen atoms at the same time. R⁴¹ and R⁴² each independently represent a hydrocarbyl group having 1 to 35 carbon atoms and optionally having an oxygen atom, a nitrogen-containing heterocyclic group having 2 to 35 carbon atoms, or a group represented by the following general formula (F1-1).

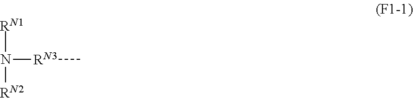

The broken line represents an attachment point.

In the general formula (F1-1), R^{N1} and R^{N2} each independently represent a hydrogen atom or a hydrocarbyl group having 1 to 20 carbon atoms and optionally having a heteroatom. R^{N1} and R^{N}2 may be bonded to each other to form a ring together with the nitrogen atom to which they are bonded. R^{N}3 represents a hydrocarbylene group having 1 to 20 carbon atoms and optionally having a heteroatom.

The hydrocarbyl group having 1 to 35 carbon atoms and represented by R⁴¹ and R⁴² may be saturated or unsaturated, and may be linear, branched, or cyclic. Specific examples thereof include: alkyl groups having 1 to 35 carbon atoms, such as a methyl group, an ethyl group, a propyl group, an isopropyl group, an n-butyl group, a sec-butyl group, a tert-butyl group, a pentyl group, a hexyl group, a heptyl group, and an octyl group; saturated cyclic hydrocarbyl groups having 1 to 35 carbon atoms, such as a cyclopentyl group, a cyclohexyl group, a cycloheptyl group, a cyclopropylmethyl group, a 4-methylcyclohexyl group, a cyclohexylmethyl group, a norbornyl group, a 1-adamantyl group, and a 1-adamantylmethyl group; groups having a steroid structure; alkenyl groups having 1 to 35 carbon atoms, such as a vinyl group and an isopropenyl group; aryl groups having 6 to 35 carbon atoms, such as a phenyl group, a 1-naphthyl group, a 2-naphthyl group, an anthryl group, alkylphenyl groups (2-methylphenyl group, a 3-methylphenyl group, a 4-methylphenyl group, a 4-ethylphenyl group, a 4-tert-butylphenyl group, a 4-n-butylphenyl group, etc.), dialkylphenyl groups (2,4-dimethylphenyl group, etc.), alkylnaphthyl groups (methylnaphthyl group, an ethylnaphthyl group, etc.), and dialkylnaphthyl groups (dialkylnaphthyl groups such as a dimethylnaphthyl group, a diethylnaphthyl group, etc.); aralkyl groups having 7 to 35 carbon atoms, such as a benzyl group, a 1-phenylethyl group, and a 2-phenylethyl group; and groups obtained by combining these.

The hydrocarbyl group may have an oxygen atom. Specific examples of the hydrocarbyl group having an oxygen atom include: saturated hydrocarbyl groups substituted with an oxo group, such as a 2-oxocyclopentyl group, a 2-oxocyclohexyl group, a 4-oxocyclohexyl group, a 2-oxopropyl group, a 2-oxoethyl group, a 2-cyclopentyl-2-oxoethyl group, a 2-cyclohexyl-2-oxoethyl group, a 2-(4-methylcyclohexyl)-2-oxoethyl group, a 4-oxa-tricyclo[4.2.1.0^{3,7}]nonan-5-on-9-yl group, and a 4-oxo-1-adamantyl group; hydroxyphenyl groups, such as a 4-hydroxyphenyl group; alkoxyphenyl groups such as a 4-methoxyphenyl group, a 3-methoxyphenyl group, a 2-methoxyphenyl group, a 4-ethoxyphenyl group, a 4-tert-butoxyphenyl group, and a 3-tert-butoxyphenyl group; alkoxynaphthyl groups, such as a methoxynaphthyl group and an ethoxynaphthyl group; dialkoxynaphthyl group, such as a dimethoxynaphthyl group and a diethoxynaphthyl group; and aryloxoalkyl groups, such as 2-aryl-2-oxoethyl groups, such as a 2-phenyl-2-oxoethyl group, a 2-(1-naphthyl)-2-oxoethyl group, and a 2-(2-naphthyl)-2-oxoethyl group.

Examples of the nitrogen-containing heterocyclic group represented by R⁴¹ and R⁴² include monovalent groups derived from aziridine, pyrrolidine, piperidine, morpholine, pyrrole, pyridine, azetine, oxazole, isoxazole, thiazole, isothiazole, imidazole, pyrazole, pyridazine, pyrimidine, pyrazine, pyrroline, 2-imidazoline, imidazolidine, 3-pyrazoline, pyrazolidine, piperazine, triazine, oxadiazine, dithiazine, indole, isoindole, quinoline, isoquinoline, cinnoline, phthalazine, quinazoline, quinoxaline, 1,8-naphthyridine, purine, pteridine, indolizine, carbazole, acridine, phenazine, phenanthridine, 1,10-phenanthroline, phenoxazine, indoline, isoindoline, quinuclidine, benzo[e]indole, benzo[cd]indole, etc.

Particularly preferable examples of R⁴¹ and R⁴² include a tert-butyl group, a cyclohexyl group, a 1-adamantyl group, a 1-adamantylmethyl group, a 4-oxa-tricyclo[4.2.1.0^{3,7}]nonan-5-on-9-yl group, a 4-oxo-1-adamantyl group, and a steroid structure-containing alkyl group.

In the general formula (F1-1), the hydrocarbyl groups having 1 to 20 carbon atoms and represented by R^{N1} and R^{N2} may be saturated or unsaturated and may be linear, branched, or cyclic. Specific examples thereof include alkyl groups having 1 to 20 carbon atoms, such as a methyl group, an ethyl group, an n-propyl group, an isopropyl group, an n-butyl group, an isobutyl group, a sec-butyl group, a tert-butyl group, a pentyl group, a hexyl group, a heptyl group, and an octyl group; a saturated cyclic hydrocarbyl groups having 3 to 20 carbon atoms, such as a cyclopentyl group, a cyclohexyl group, a cycloheptyl group, a cyclopropylmethyl group, a 4-methylcyclohexyl group, a cyclohexylmethyl group, a norbornyl group, and an adamantyl group; alkenyl groups having 2 to 20 carbon atoms, such as a vinyl group, a 1-propenyl group, a 2-propenyl group, a butenyl group, and a hexenyl groups; unsaturated cyclic hydrocarbyl groups having 3 to 20 carbon atoms, such as a cyclohexenyl group; aryl groups having 6 to 20 carbon atoms, such as a phenyl group, a naphthyl group, and a thienyl group; aralkyl groups having 7 to 20 carbon atoms, such as a benzyl group, an 1-phenylethyl group, and a 2-phenylethyl group; and groups obtained by combining these groups. Furthermore, part or all of the hydrogen atoms of the hydrocarbyl group may be substituted with a group having a heteroatom, such as an oxygen atom, a sulfur atom, a nitrogen atom, or a halogen atom, or part of the —CH₂— of the hydrocarbyl group may be substituted with a group having a heteroatom, such as an oxygen atom, a sulfur atom, or a nitrogen atom. As a result, the hydrocarbyl group may have a hydroxy group, a fluorine atom, a chlorine atom, a bromine atom, an iodine atom, a cyano group, a carbonyl group, an ether bond, an ester bond, a sulfonate ester bond, a carbonate bond, a lactone ring, a sultone ring, a carboxylic anhydride (—C(═O)—O—C(═O)—), a haloalkyl group, or the like.

When R^{N1} and R^{N2} are bonded to each other to form a ring together with the nitrogen atom to which they are bonded, specific examples of the ring include aziridine, pyrrolidine, piperidine, morpholine, pyrrole, pyridine, azetine, oxazole, isoxazole, thiazole, isothiazole, imidazole, pyrazole, pyridazine, pyrimidine, pyrazine, pyrroline, 2-imidazoline, imidazolidine, 3-pyrazoline, pyrazolidine, piperazine, triazine, oxadiazine, dithiazine, indole, isoindole, quinoline, isoquinoline, cinnoline, phthalazine, quinazoline, quinoxaline, 1,8-naphthyridine, purine, pteridine, indolizine, carbazole, acridine, phenazine, phenanthridine, 1,10-phenanthroline, phenoxazine, indoline, isoindoline, quinuclidine, benzo[e]indole, and benzo[cd]indole. In addition, part or all of the hydrogen atoms of these rings may be substituted with a hydrocarbyl group having 1 to 20 carbon atoms or a group having a heteroatom, such as an oxygen atom, a sulfur atom, a nitrogen atom or a halogen atom, and part of the —CH₂— of these rings may be substituted with a group having a heteroatom such as an oxygen atom, a sulfur atom, or a nitrogen atom. As a result, the ring may have a hydroxy group, a fluorine atom, a chlorine atom, a bromine atom, an iodine atom, a cyano group, a carbonyl group, an ether bond, an ester bond, a sulfonate ester bond, a carbonate bond, a lactone ring, a sultone ring, a carboxylic anhydride (—C(═O)—O—C(═O)—), a haloalkyl group, or the like.

In the general formula (F1-1), examples of the hydrocarbylene group represented by R^{N3} include: linear alkanediyl groups having 1 to 20 carbon atoms a methylene group, an ethylene group, a propane-1,3-diyl group, a butane-1,4-diyl group, a pentane-1,5-diyl group, a hexane-1,6-diyl group, a heptane-1,7-diyl group, an octane-1,8-diyl group, a nonane-1,9-diyl group, a decane-1,10-diyl group, an undecane-1,11-diyl group, a dodecane-1,12-diyl group, a tridecane-1,13-diyl group, a tetradecane-1,14-diyl group, a pentadecane-1,15-diyl group, a hexadecane-1,16-diyl group, and a heptadecane-1,17-diyl group; branched alkanediyl groups having 2 to 20 carbon atoms and having a structure in which part of the hydrogen atoms of the above-mentioned linear alkanediyl group have been substituted with alkyl groups, such as a methyl group, an ethyl group, a propyl group, an isopropyl group, a butyl group, a sec-butyl group, and a tert-butyl groups; saturated cyclic hydrocarbyl groups having 3 to 20 carbon atoms, such as a cyclopentanediyl group, a cyclohexanediyl group, a norbornanediyl group, and a adamantanediyl group; arylene groups, such as a phenylene group and a naphthylene group; and groups obtained by combining these. In addition, part or all of the hydrogen atoms of the hydrocarbylene group may be substituted with a group having a heteroatom, such as an oxygen atom, a sulfur atom, a nitrogen atom, or a halogen atom, or part of the —CH₂— groups of the hydrocarbylene group may be substituted with a group having a heteroatom, such as an oxygen atom, a sulfur atom, or a nitrogen atom. As a result, the hydrocarbylene group may have a hydroxy group, a fluorine atom, a chlorine atom, a bromine atom, an iodine atom, a cyano group, a carbonyl group, an ether bond, an ester bond, a sulfonate ester bond, a carbonate bond, a lactone ring, a sultone ring, a carboxylic acid anhydride (—C(═O)—O—C(═O)—), a haloalkyl group, or the like.

Specific examples of the anion of the photoacid generator represented by the general formula (F1) include, but are not limited to, those described in paragraphs [0117] to [0118] of JP7240301B2.

Specific examples of the anion of the photoacid generator represented by the general formula (F2) include, but are not limited to, those described in paragraphs [0126] to [0127] of JP7240301B2.

In the general formulae (F1) and (F2), M_{A}⁺ represents an onium cation. The onium cation is preferably a sulfonium cation represented by the general formula (Z-1) or an iodonium cation represented by the general formula (Z-2).

Furthermore, those represented by the following general formula (F3) are also preferable as the other photoacid generator.

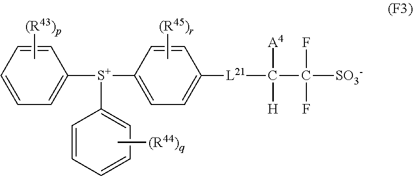

In the general formula (F3), “p” and “q” each independently represent an integer of 0 to 5. “r” represents an integer of 0 to 4. A4 represents a hydrogen atom or a trifluoromethyl group. R⁴³, R⁴⁴, and R⁴⁵ each independently represent a hydrocarbyl group having 1 to 20 carbon atoms and optionally having a heteroatom. L^{C} represents a single bond, an ether bond, or a hydrocarbylene group having 1 to 20 carbon atoms and optionally having a heteroatom.

The hydrocarbyl groups represented by R⁴³, R⁴⁴, and R⁴⁵ may be saturated or unsaturated and may be linear, branched, or cyclic. Specific examples thereof include: alkyl groups having 1 to 20 carbon atoms, such as a methyl group, an ethyl group, an n-propyl group, an isopropyl group, an n-butyl group, a sec-butyl group, a tert-butyl group, an n-pentyl group, a tert-pentyl group, an n-hexyl group, an n-octyl group, an n-nonyl group, and an n-decyl group; and saturated cyclic hydrocarbyl groups having 3 to 20 carbon atoms, such as a cyclopentyl group, a cyclohexyl group, a 2-ethylhexyl, cyclopentylmethyl group, a cyclopentylethyl group, a cyclopentylbutyl group, a cyclohexylmethyl group, a cyclohexylethyl group, a cyclohexylbutyl group, a norbornyl group, an oxanorbornyl group, a tricyclo[5.2.1.0^{2,6}]decyl group, and an adamantyl group. Furthermore, part or all of the hydrogen atoms of the hydrocarbyl group may be substituted with a group having a heteroatom, such as an oxygen atom, a sulfur atom, a nitrogen atom, or a halogen atom, and part of the —CH₂— of the hydrocarbyl group may be substituted with a group having a heteroatom, such as an oxygen atom, a sulfur atom, or a nitrogen atom. As a result, the hydrocarbyl group may have a hydroxy group, a fluorine atom, a chlorine atom, a bromine atom, an iodine atom, a cyano group, a carbonyl group, an ether bond, an ester bond, a sulfonic acid ester bond, a carbonate bond, a lactone ring, a sultone ring, a carboxylic acid anhydride (—C(═O)—O—C(═O)—), a haloalkyl group, etc. As R⁴³, R⁴⁴, and R⁵, a methyl group, a methoxy group, a tert-butyl group, a tert-butoxy group, or the like is preferable.

In the general formula (F3), examples of the hydrocarbylene group represented by L^{C} include the same groups as those exemplified as the hydrocarbylene groups represented by R^{N3} in the description of the general formula (F1-1). In addition, part or all of the hydrogen atoms of the hydrocarbylene group may be substituted with a group having a heteroatom such as an oxygen atom, a sulfur atom, a nitrogen atom, or a halogen atom, and part of the —CH₂— of the hydrocarbylene group may be substituted with a group having a heteroatom such as an oxygen atom, a sulfur atom, or a nitrogen atom. As a result, the hydrocarbylene group may have a hydroxy group, a fluorine atom, a chlorine atom, a bromine atom, an iodine atom, a cyano group, a carbonyl group, an ether bond, an ester bond, a sulfonate ester bond, a carbonate bond, a lactone ring, a sultone ring, a carboxylic anhydride (—C(═O)—O—C(═O)—), a haloalkyl group, or the like.

Specific examples of the photoacid generator represented by the general formula (F3) include those described in paragraphs [0123] to [0124] of JP7240301B2, but are not limited thereto.

Among the other photoacid generators, compounds having a fluorine atom-containing group, such as a trifluoromethyl group, as an anion are highly hydrophobic and exhibit low elution in immersion water. Furthermore, such a compound has a fluorine atom-containing group and therefore has high solvent solubility, enabling reduced residues after organic solvent development. This can reduce defects after development; therefore, such a compound is suitable as a chemically-amplified resist composition for ArF immersion exposure.

Other specific examples of the other photoacid generators include the compounds described in paragraphs [0122] to [0142] of JP2008-111103A. Particularly preferable examples include the compounds described in paragraphs [0088] to [0092] of JP2014-001259A, the compounds described in paragraphs [0015] to [0017] of JP2012-41320A, and the compounds described in paragraphs [0015] to [0029] of JP2012-106986A. The partially fluorinated sulfonic acid-generating photoacid generators described in the above publications generate acids with suitable strength and suitable diffusion length. They are therefore preferably used, particularly in ArF lithography.

When the inventive chemically-amplified resist composition includes the component (F) other photoacid generator, the contained amount thereof is preferably 0.1 to 50 parts by mass, more preferably 0.2 to 40 parts by mass, and further preferably 0.3 to 35 parts by mass, relative to 80 parts by mass of the component (B) base polymer. When the amount of the other photoacid generator (F) contained is within the above range, resolution is not deteriorated, and there is no risk of causing a problem of foreign matter after development or during stripping of the resist film. When the compound represented by the general formula (F2) is added, the contained amount thereof is preferably 0 to 50 mass % relative to the total amount of the components (A) and (F).

### [(G) Surfactant]

The inventive chemically-amplified resist composition may further contain a surfactant as the component (G). The component (G) surfactant is preferably: insoluble or hardly soluble in water and soluble in alkaline developer; or insoluble or hardly soluble in water and alkaline developer. As such a surfactant, components defined as the surfactants (S) in JP2010-215608A and JP2011-16746A can be referred to.

Among the surfactants described in the above patent publications, the surfactant that is insoluble or hardly soluble in water and alkaline developer is preferably FC-4430 (manufactured by 3M Company), SURFLON (registered trademark)S-381 (manufactured by AGC Seimi Chemical Co., Ltd.), OLFIN (registered trademark) E1004 (manufactured by Nissin Chemical Industry Co., Ltd.), KH-20 and KH-30 (manufactured by AGC Seimi Chemical Co., Ltd.), and an oxetane ring-opening polymerized product represented by the following general formula (surf-1).

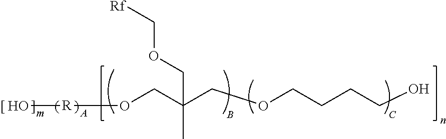

Here, regardless of the above description, R, Rf, A, B, C, “m”, and “n” apply only to the general formula (surf-1). R represents a divalent to tetravalent aliphatic group having 2 to 5 carbon atoms. Examples of the aliphatic group include divalent groups, such as an ethylene group, a 1,2-propanediyl group, a 1,4-butanediyl group, a 2,2-dimethyl-1,3-propanediyl group, and a 1,5-pentanediyl group, and examples of trivalent or tetravalent groups include those shown below.

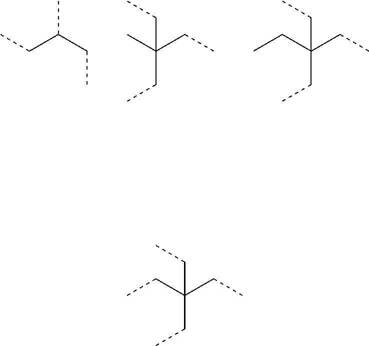

In the formulae, the broken lines represent attachment points and are partial structures derived from glycerol, trimethylolethane, trimethylolpropane, and pentaerythritol, respectively.

Among these, a 1,4-butanediyl group or a 2,2-dimethyl-1,3-propanediyl group is preferred.

In the general formula (surf-1), Rf represents a trifluoromethyl group or a pentafluoroethyl group, and is preferably a trifluoromethyl group. “m” represents an integer of 0 to 3. “n” represents an integer of 1 to 4. The sum of “n” and “m”, which represents a valency of “R”, represents an integer of 2 to 4. “A” represents 1. “B” represents an integer of 2 to 25, and preferably represents an integer of 4 to 20. “C” represents an integer of 0 to 10, and preferably is 0 or 1. The general formula (surf-1) does not stipulate the array order of its constituting units, and the units may be bonded in a block or randomly. A manufacturing method for the surfactant of partially fluorinated oxetane ring-opening polymerized product is described in detail in U.S. Pat. No. 5,650,483A1.

### [Patterning Process]

The inventive patterning process includes the steps of: forming a resist film on a substrate by using the chemically-amplified resist composition described above; exposing the resist film by using a high-energy radiation; and developing the exposed resist film by using a developer.

As the substrate, it is possible to use, for example, a substrate for manufacturing integrated circuits (Si, SiO₂, SiN, SiON, TiN, WSi, BPSG, SOG, organic antireflective film, etc.) or a substrate for manufacturing mask circuits (Cr, CrO, CrON, MoSi₂, SiO₂, etc.).

The resist film can be formed, for example, by applying the inventive chemically-amplified resist composition onto a substrate by a method, such as spin coating, so that the film thickness is 0.05 to 2 μm, and then pre-baking the applied composition on a hot plate preferably at 60 to 150 °C for 1 to 10 minutes, more preferably at 80 to 140 °C for 1 to 5 minutes.

Examples of the high-energy radiation used to expose the resist film include KrF excimer laser light, ArF excimer laser light, EUV with a wavelength of 3 to 15 nm, and EB. When KrF excimer laser light, ArF excimer laser light, or EUV is used, exposure can be performed with a mask to form the desired pattern by irradiating so that an exposure dose will be preferably 1 to 200 mJ/cm², more preferably 10 to 100 mJ/cm². When EB is used, exposure is performed with a mask to form the desired pattern or directly so that an exposure dose will be preferably 0.1 to 100 μC/cm², more preferably 0.5 to 50 μC/cm².

The exposure can be performed, in addition to a common exposure method, by using an immersion method in which a liquid having a refractive index of 1.0 or more is interposed between a resist film and a projection lens. In this case, a protective film insoluble in water can also be used.

The protective film, insoluble in water, is used to prevent an eluted material from the resist film and to improve the water-slipping property on the film surface, and roughly falls into two types. One is an organic-solvent stripping type, in which the protective film is required to be stripped with an organic solvent that does not dissolve the resist film before development with an aqueous alkaline solution. The other is an aqueous alkaline solution-soluble type, in which the protective film is soluble in an alkaline developer and is removed together with the removal of a soluble portion in the resist film. The latter is particularly preferably a material that is based on a polymer having a 1,1,1,3,3,3-hexafluoro-2-propanol residue, which is insoluble in water and soluble in the alkaline developer, and dissolved in an alcoholic solvent having 4 or more carbon atoms, an ether solvent having 8 to 12 carbon atoms, and a mixed solvent thereof. The protective film may also be a material in which the aforementioned surfactant, insoluble in water and soluble in an alkaline developer, is dissolved in the alcoholic solvent having 4 or more carbon atoms, the ether solvent having 8 to 12 carbon atoms, or the mixed solvent thereof.

After the exposure, post-exposure baking (PEB) may be performed. PEB may be performed, for example, by heating on a hot plate at preferably 60 to 150 °C for 1 to 5 minutes, more preferably 80 to 140 °C for 1 to 3 minutes.

The development can be performed, for example, using a developer of an alkaline aqueous solution of tetramethylammonium hydroxide (TMAH) or the like, preferably at a concentration of 0.1 to 5 mass %, more preferably 2 to 3% by mass; for preferably 0.1 to 3 minutes, more preferably 0.5 to 2 minutes; and by a conventional method such as a dip method, a puddle method, or a spray method. Thereby, a desired pattern can be formed on the substrate.

After forming the resist film, the resist film may be rinsed with pure water to extract an acid generator or the like from the film surface or to wash away particles, or the resist film may be rinsed after exposure to remove water remaining on the film.

In the inventive patterning process, negative-tone development that dissolve the unexposed areas can be performed by using an organic solvent instead of the alkaline aqueous solution as the developer.

As a developer for this development using an organic solvent, it is possible to use 2-octanone, 2-nonanone, 2-heptanone, 3-heptanone, 4-heptanone, 2-hexanone, 3-hexanone, diisobutyl ketone, methylcyclohexanone, acetophenone, methylacetophenone, propyl acetate, butyl acetate, isobutyl acetate, pentyl acetate, butenyl acetate, isopentyl acetate, phenyl acetate, propyl formate, butyl formate, isobutyl formate, pentyl formate, isopentyl formate, methyl valerate, methyl pentenoate, methyl crotonate, ethyl crotonate, methyl lactate, ethyl lactate, propyl lactate, butyl lactate, isobutyl lactate, pentyl lactate, isopentyl lactate, methyl 2-hydroxyisobutyrate, ethyl 2-hydroxyisobutyrate, methyl benzoate, ethyl benzoate, benzyl acetate, methyl Phenylacetate, benzyl formate, phenylethyl formate, methyl 3-phenylpropionate, benzyl propionate, ethyl Phenylacetate, 2-phenylethyl acetate, etc. One of these organic solvents may be used, or two or more thereof may be used in mixture.

### EXAMPLE

Hereinafter, the present invention will be specifically described with reference to Synthesis Example, Examples, and Comparative Examples. However, the present invention is not limited thereto. It should be understood that the following apparatus was used.
MALDI TOF-MS: S3000 manufactured by JEOL Ltd.

### [1] Synthesis of Onium Salt

### [Example 1-1] Synthesis of Onium Salt PAG-1

Synthesis of PAG-1 was performed according to the following reaction formula.

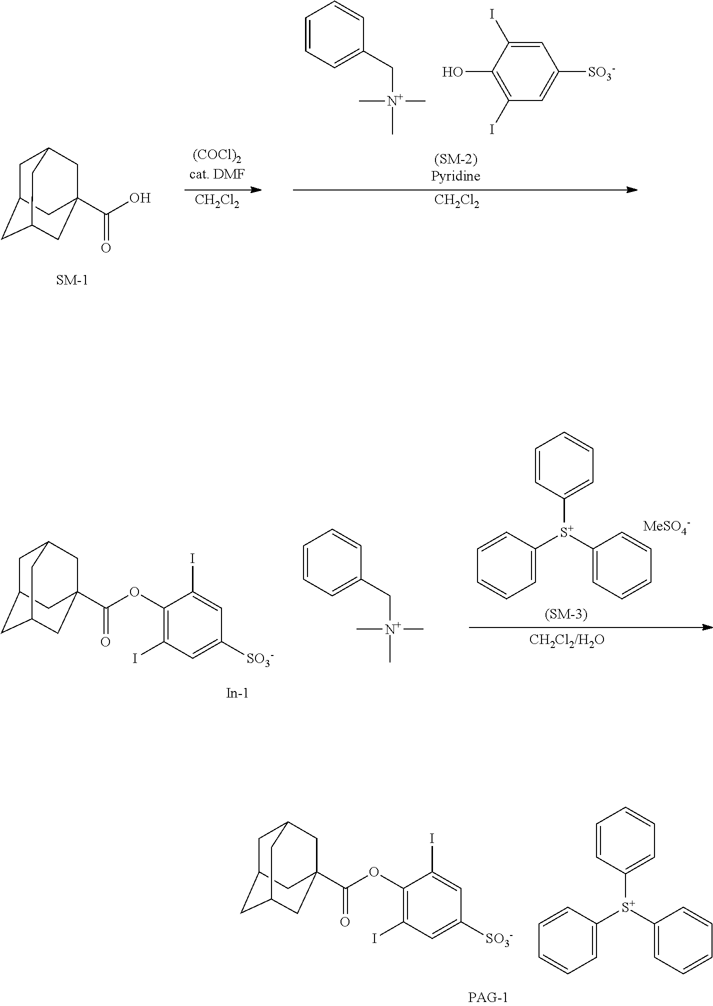

### (1) Synthesis of Intermediate In-1

Under a nitrogen atmosphere, a material SM-1 (18.0 g), oxanyl chloride (15.1 g), DMF (N,N-dimethylformamide) (0.2 g), and methylene chloride (100 g) were added to a reaction vessel. The temperature inside the reaction vessel was raised to 35 °C, and aging was performed for 3 hours. After the aging, the temperature of the mixture was lowered to room temperature, and the solvent was distilled off to obtain a solid. The obtained solid was dissolved in methylene chloride (60 g). Furthermore, the mixture was added with a material SM-2 (74.7 g) and pyridine (3.1 g) and was cooled in an ice bath. After the addition, the temperature was raised to room temperature, and aging was performed for 12 hours. After the aging, water was added to terminate the reaction. Subsequently, a common aqueous workup was performed, and the solvent was distilled off. After that, diisopropyl ether was added to wash the residue, and thus, 70.0 g of intermediate In-1 was obtained as a crystalline product (95% yield).

### (2) Synthesis of PAG-1

Under a nitrogen atmosphere, the intermediate In-1 (70.0 g), a material SM-3 (56.1 g), methylene chloride (100 g), and water (50 g) were added and stirred for 15 minutes. After that, the organic layer was collected, washed with water, and then concentrated under reduced pressure. Diisopropyl ether (50 g) was added to the concentrated liquid to perform crystallization, and thus, 79.2 g of onium salt PAG-1, being the target product, was obtained as a white crystalline product (98% yield). The measurement results of the obtained onium salt PAG-1 are as follows.

### MALDI TOF-MS:

POSITIVE M+ 263 (C₁₈H₁₅S⁺ equivalent)
NEGATIVE M− 587 (C₁₇H₁₇I₂O₅S⁻ equivalent)

### [Example 1-2 to 1-9] Synthesis of Onium Salts PAG-2 to PAG-9

Onium salts PAG-2 to PAG-9, represented by the following formulae, were synthesized using corresponding materials by using publicly known organic synthesis reactions.

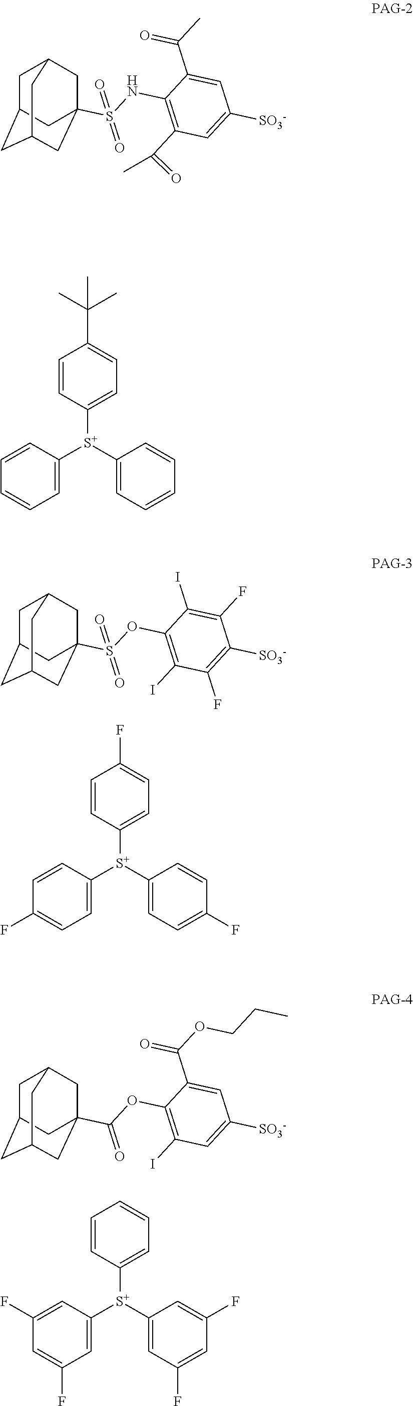

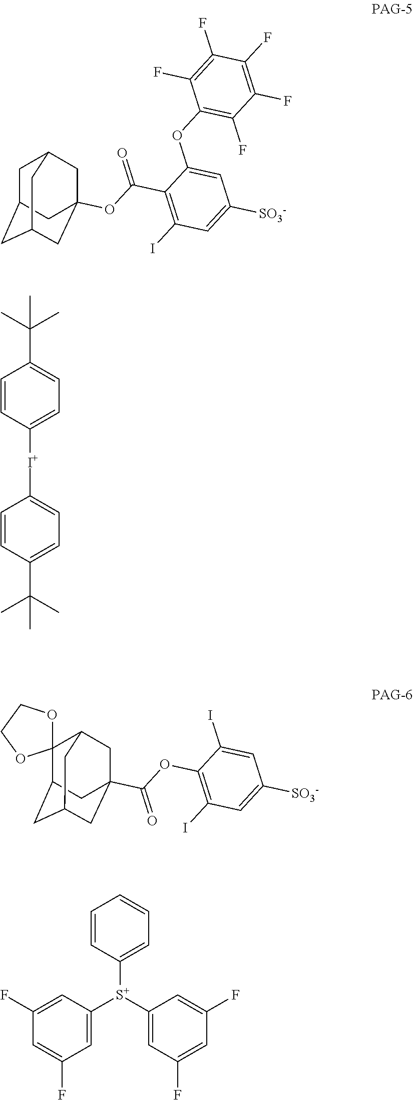

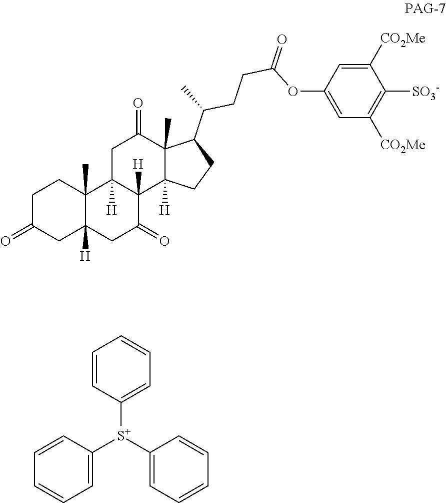

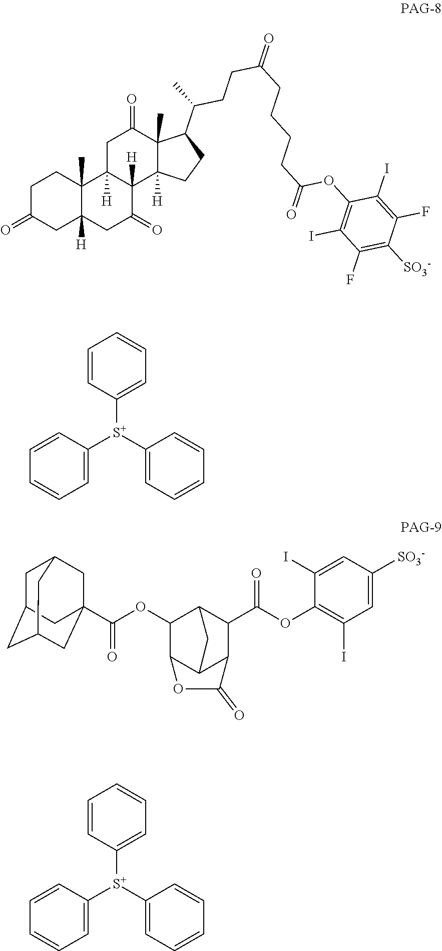

### [2] Synthesis of Polymers

Polymers used for the inventive chemically-amplified resist compositions were synthesized by the method shown below. It should be understood that the Mw of the obtained polymer was measured as a value in terms of polystyrene by GPC using THF as the eluent.

### [Synthesis Example 1] Synthesis of Polymer P-1

Under a nitrogen atmosphere, α-methacryloxy-γ-butyrolactone (32.5 g), 3-hydroxyadamantyl methacrylate (12.9 g), 1-ethylcyclopentyl methacrylate (54.6 g), and dimethyl 2,2′-azobisisobutyrate (6.27 g) were dissolved in PGMEA (155 g) to prepare a solution. The solution was added dropwise over 5 hours to PGMEA (78 g) that had been stirred at 80 °C, under a nitrogen atmosphere. After the dropwise addition, the reaction solution was stirred for 2 hours while maintaining the temperature at 80 °C, and then cooled to room temperature. After that, the reaction solution was added dropwise to methanol (2,000 g). The precipitated solid was filtered and dried in a vacuum at 50 °C for 20 hours to obtain polymer P-1, shown below, as a white powder. The yield was 82 g (82%).

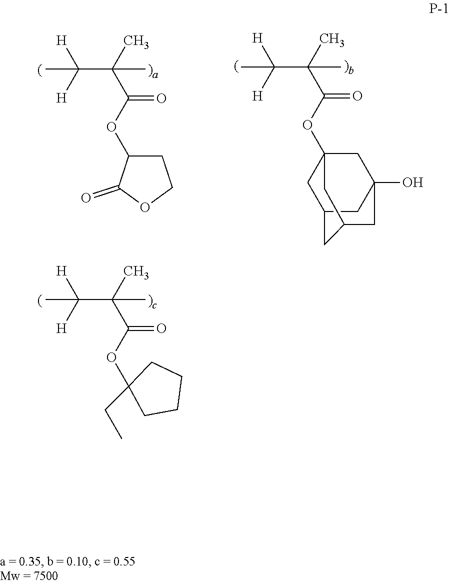

### [Synthesis Example 2] Synthesis of Polymer P-2

Polymer P-2, shown below, was produced in the same manner as in Synthesis Example 1, except that the kinds and blending ratios of the monomers were changed.

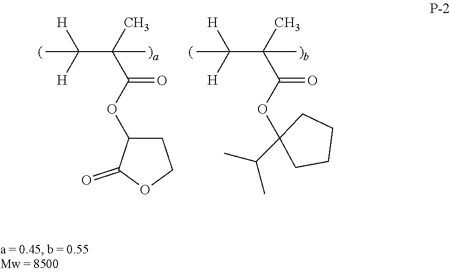

### [3] Preparation of Chemically-Amplified Resist

### Compositions and Evaluation Thereof

### Examples 2-1 to 2-13 and Comparative Examples 1-1 to 1-5

### (1) Preparation of Chemically-Amplified Resist Compositions

The predetermined components selected from the inventive onium salts (PAG-1 to PAG-9), comparative photoacid generators (cPAG-1 to cPAG-4), polymers (P-1 and P-2), quenchers (Q-1 to Q-4), and a fluorine-atom-containing polymer (SF-1) at the composition shown in the following Table 1 were dissolved in a solvent containing 0.01 mass % of the surfactant A (manufactured by OMNOVA Solutions Inc.) to prepare solutions. The solutions were filtered with a 0.2-μm filter made of Teflon (registered trademark) to prepare chemically-amplified resist compositions.

It should be understood that the solvents, the comparative photoacid generators (cPAG-1 to cPAG-4), the quenchers (Q-1 to Q-4), and the fluorine-atom-containing polymer (SF-1) were as follows.

### Solvents:

PGMEA (propylene glycol monomethyl ether acetate)
GBL (γ-butyrolactone)

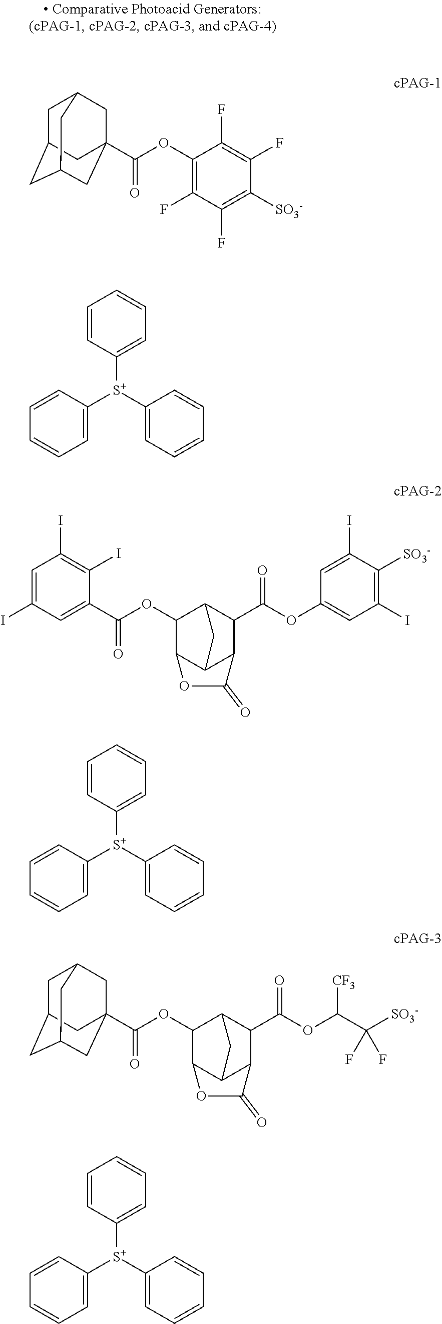

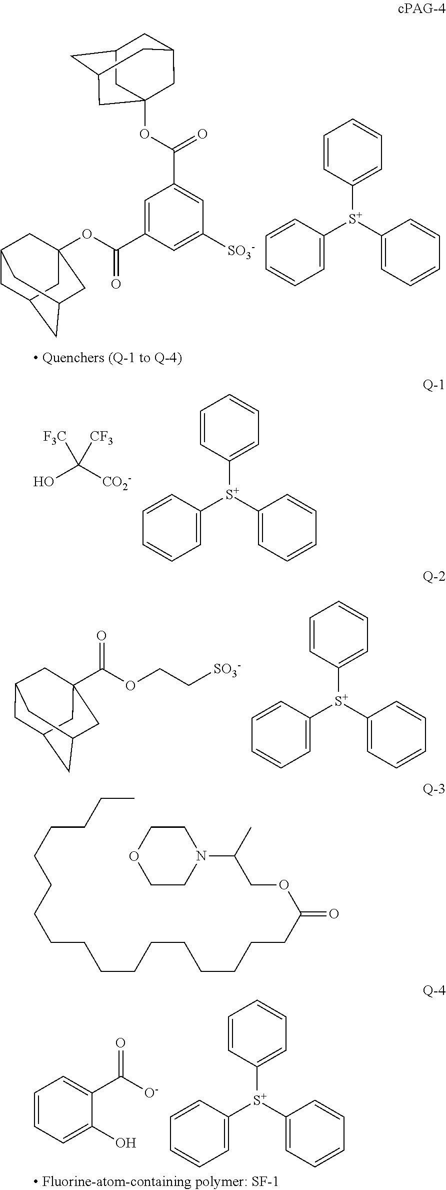

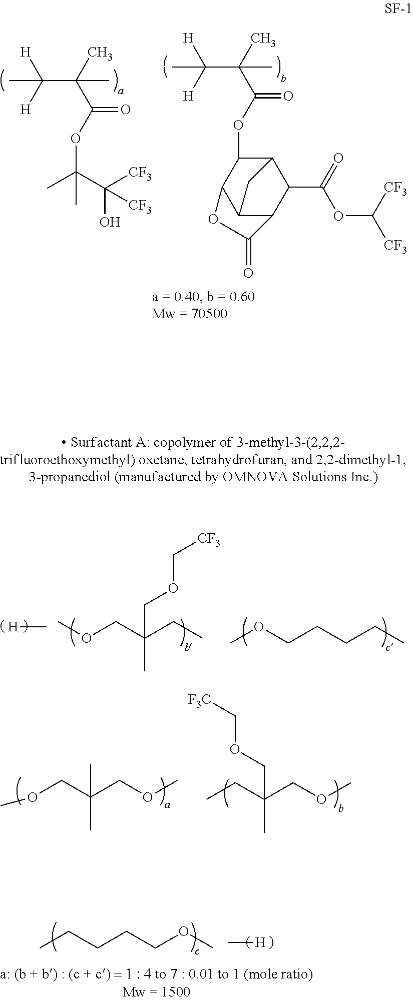

### (2) ArF Lithography Evaluation

A spin-on carbon film, ODL-180 (carbon content of 80 mass %), manufactured by Shin-Etsu Chemical Co., Ltd., with a film thickness of 180 nm, was formed on a silicon substrate. A silicon-containing spin-on hard mask, SHB-A941 (silicon content of 43 mass %), with a film thickness of 35 nm, was formed on the spin-on carbon film to obtain a substrate for a tri-layer process. Each of the chemically-amplified resist compositions was applied by spin-coating on the substrate, and the coated substrate was baked on a hot plate at 100 °C for 60 seconds to form a resist film with a film thickness of 100 nm. The resist film was exposed using an ArF excimer laser immersion scanner (XT-1900Gi manufactured by ASML Holding N.V., NA=1.35, 3/4 Annular, X-Y polarization) for a contact hole pattern (CH pattern) with an on-wafer size of 45 nm and a pitch of 110 nm while changing the exposure dose and focus (exposure dose pitch: 1 mJ/cm², focus pitch: 0.025 μm). After the exposure, PEB was performed at the temperature shown in Table 1 for 60 seconds. As the immersion liquid, water was used. Thereafter, puddle development was performed with n-butyl acetate for 30 seconds, rinse with 4-methyl-2-pentanol was performed, and spin-drying was performed to obtain a CH pattern of a negative pattern. The obtained CH pattern was observed with a CD-SEM (CG5000) manufactured by Hitachi High-Tech Corporation to evaluate sensitivity, CDU, and MEF in accordance with the following methods. The results are shown together in Table 1.

### [Sensitivity Evaluation]

The optimum exposure dose Eop (mJ/cm²) to yield the CH pattern with a hole pitch of 104 nm and a hole diameter of 52 nm was determined and defined as the sensitivity.

### [CDU Evaluation]

Sizes of the CH patterns obtained by irradiation at Eop were measured at 10 locations (9 CH patterns per location) exposed at the same exposure dose. Based on the results, the tripled value (3σ) of the standard deviation (σ) was determined as CDU. The smaller this value, the better the CDU of the CH pattern.

### [MEF Evaluation]

Patterns were formed by irradiating at the Eop with a fixed pitch and varying the size on a mask, and the line width of each pattern was measured. The inclination of the line width of the pattern relative to the size on the mask was defined as MEF. MEF of 3.5 or less was evaluated as favorable.

TABLE 1

| | | | | Fluorine | | | | | |
|---|---|---|---|---|---|---|---|---|---|
| | | | | atom- | | | | | |
| | | Photoacid | | containing | | | | | |
| | Polymer | generator | Quencher | polymer | | | | | |
| | (parts by | (parts by | (parts by | (parts by | Organic solvent | PEB | Sensitivity | CDU | |
| | mass) | mass) | mass) | mass) | (parts by mass) | (°C) | (mJ/cm²) | (nm) | MEF |
| Example | P-1 | PAG-1 | Q-1 | SF-1 | PGMEA(1550) | 95 | 30 | 4.2 | 2.9 |
| 2-1 | (80) | (8.0) | (4.5) | (5.0) | GBL(250) | | | | |
| Example | P-1 | PAG-2 | Q-1 | SF-1 | PGMEA(1550) | 95 | 41 | 4.1 | 3.2 |
| 2-2 | (80) | (8.0) | (4.5) | (5.0) | GBL(250) | | | | |
| Example | P-1 | PAG-3 | Q-1 | SF-1 | PGMEA(1550) | 95 | 32 | 4.0 | 3.0 |
| 2-3 | (80) | (8.0) | (4.5) | (5.0) | GBL(250) | | | | |
| Example | P-1 | PAG-4 | Q-1 | SF-1 | PGMEA(1550) | 95 | 36 | 4.2 | 3.1 |
| 2-4 | (80) | (8.0) | (4.5) | (5.0) | GBL(250) | | | | |
| Example | P-1 | PAG-5 | Q-1 | SF-1 | PGMEA(1550) | 95 | 38 | 4.5 | 3.2 |
| 2-5 | (80) | (8.0) | (4.5) | (5.0) | GBL(250) | | | | |
| Example | P-1 | PAG-6 | Q-1 | SF-1 | PGMEA(1550) | 95 | 32 | 3.9 | 2.9 |
| 2-6 | (80) | (8.0) | (4.5) | (5.0) | GBL(250) | | | | |
| Example | P-1 | PAG-7 | Q-1 | SF-1 | PGMEA(1550) | 95 | 35 | 3.4 | 2.8 |
| 2-7 | (80) | (8.0) | (4.5) | (5.0) | GBL(250) | | | | |
| Example | P-1 | PAG-8 | Q-1 | SF-1 | PGMEA(1550) | 95 | 31 | 3.6 | 2.8 |
| 2-8 | (80) | (8.0) | (4.5) | (5.0) | GBL(250) | | | | |
| Example | P-1 | PAG-9 | Q-1 | SF-1 | PGMEA(1550) | 95 | 29 | 3.5 | 2.8 |
| 2-9 | (80) | (8.0) | (4.5) | (5.0) | GBL(250) | | | | |
| Example | P-1 | PAG-1 | Q-2 | SF-1 | PGMEA(1550) | 95 | 32 | 4.1 | 2.9 |
| 2-10 | (80) | (8.0) | (4.5) | (5.0) | GBL(250) | | | | |
| Example | P-1 | PAG-1 | Q-3 | SF-1 | PGMEA(1550) | 95 | 33 | 4.1 | 3.5 |
| 2-11 | (80) | (8.0) | (2.0) | (5.0) | GBL(250) | | | | |
| Example | P-2 | PAG-1 | Q-1 | SF-1 | PGMEA(1550) | 95 | 32 | 3.6 | 3.3 |
| 2-12 | (80) | (8.0) | (4.5) | (5.0) | GBL(250) | | | | |
| Example | P-1 | PAG-1 | Q-4 | SF-1 | PGMEA(1550) | 95 | 33 | 4.1 | 2.9 |
| 2-13 | (80) | (8.0) | (4.5) | (5.0) | GBL(250) | | | | |
| Comparative | P-1 | cPAG-1 | Q-1 | SF-1 | PGMEA(1550) | 95 | 42 | 6.1 | 4.2 |
| Example | (80) | (8.0) | (4.5) | (5.0) | GBL(250) | | | | |
| 1-1 | | | | | | | | | |
| Comparative | P-1 | cPAG-2 | Q-1 | SF-1 | PGMEA(1550) | 95 | 50 | 6.2 | 4.0 |
| Example | (80) | (8.0) | (4.5) | (5.0) | GBL(250) | | | | |
| 1-2 | | | | | | | | | |
| Comparative | P-1 | cPAG-3 | Q-1 | SF-1 | PGMEA(1550) | 95 | 43 | 5.0 | 4.5 |
| Example | (80) | (8.0) | (4.5) | (5.0) | GBL(250) | | | | |
| 1-3 | | | | | | | | | |
| Comparative | P-1 | cPAG-4 | Q-4 | SF-1 | PGMEA(1550) | 95 | 52 | 5.5 | 5.1 |
| Example | (80) | (8.0) | (4.5) | (5.0) | GBL(250) | | | | |
| 1-4 | | | | | | | | | |
| Comparative | P-1 | cPAG-1 | Q-4 | SF-1 | PGMEA(1550) | 95 | 43 | 5.5 | 5.1 |
| Example | (80) | (8.0) | (4.5) | (5.0) | GBL(250) | | | | |
| 1-5 | | | | | | | | | |

From the results shown in Table 1, it was shown that Examples 2-1 to 2-13 using the chemically-amplified resist compositions containing the inventive onium salts as a photoacid generator had favorable sensitivity, were excellent also in CDU and MEF, and were suitable as a material for ArF immersion lithography. On the other hand, the resist compositions (Comparative Examples 1-1 to 1-5) containing the conventional photoacid generator were insufficient in either sensitivity, MEF, or CDU. From the above, it was shown that the chemically-amplified resist compositions containing the inventive onium salts as a photoacid generator were useful for ArF immersion lithography. It should be understood that in the comparative photoacid generators: cPAG-1 does not have R⁰; cPAG-2 involves resonance structures in W²; cPAG-3 does not have an aromatic ring in the anion; and cPAG-4 has m2=1 and m3=0, which does not satisfy the condition 2≤m2+m3≤4, therefore, cPAG-1 to cPAG-4 do not satisfy the general formula (a) of the inventive onium salts.

The present description includes the following embodiments.

[1]: An onium salt represented by the following general formula (a),

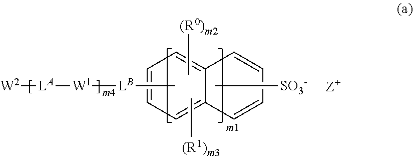

wherein
“m1” represents 0 to 1; “m2” represents an integer of 1 to 4 when “m1” is 0, “m2” represents an integer of 1 to 6 when “m1” is 1, “m3” represents an integer of 0 to 3 when “m1” is 0, “m3” represents an integer of 0 to 5 when “m1” is 1, provided that 2≤m2+m3≤4 when “m1” is 0 and 2≤m2+m3≤6 when “m1” is 1; and “m4” represents an integer of 0 to 2;
R⁰ represents an electron-withdrawing group other than a fluorine atom, and R⁰s may be the same as or different from each other when “m2” is 2 or more;
R¹ represents a fluorine atom, a hydroxy group, or a hydrocarbyl group having 1 to 20 carbon atoms and optionally having a heteroatom, R¹s may be the same as or different from each other when “m3” is 2 or more, and furthermore multiple R¹s may be bonded to each other to form a ring together with the carbon atoms to which they are bonded when “m3” is 2 or more;
L^{A} and L^{B} each independently represent a single bond, an ether bond, an ester bond, an amide bond, a sulfonate ester bond, a sulfonamide bond, a carbonate bond, or a carbamate bond;
W¹ represents a hydrocarbylene group having 1 to 40 carbon atoms, optionally having a heteroatom, and not involving resonance structures; W² represents a hydrocarbyl group having 1 to 40 carbon atoms, optionally having a heteroatom, and not involving resonance structures; and
Z⁺ represents a sulfonium cation represented by the following general formula (Z-1) or an iodonium cation represented by the following general formula (Z-2),

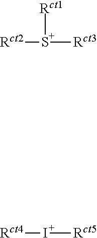

wherein
R^{ct1} to R^{ct5} each independently represent a halogen atom or a hydrocarbyl group having 1 to 30 carbon atoms and optionally having a heteroatom, and R^{ct1} and R^{ct2} may be bonded to each other to form a ring together with the sulfur atom to which they are bonded.
   [2]: The onium salt of the above [1] wherein at least one of W¹ and W² in the general formula (a) have an alicyclic structure having 3 to 40 carbon atoms and optionally having a hetero atom.
   [3]: The onium salt of the above [1] or [2], represented by the following general formula (a1),

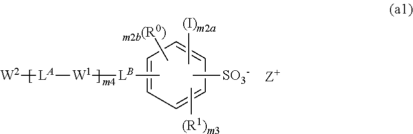

wherein “m3”, “m4”, L^{A}, L^{B}, R⁰, R¹, W¹, W², and Z⁺ represent the same as defined above, “m2a” represents an integer of 1 to 4, and “m2b” represents an integer of 0 to 3.
   [4]: The onium salt of any one of the above [1] to [3], represented by the following general formula (a2),

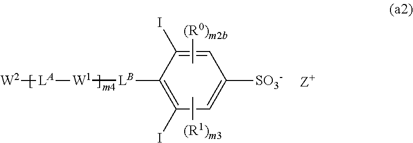

wherein “m4”, L^{A}, L^{B}, R⁰, R¹, W¹, W², and Z⁺ represent the same as defined above, and “m2b” and “m3” represent an integer of 0 to 2.
   [5]: A photoacid generator comprising the onium salt of any one of the above [1] to [4].
   [6]: A chemically-amplified resist composition comprising the photoacid generator (A) of the above [5].
   [7]: The chemically-amplified resist composition of the above [6], further comprising a base polymer having a repeating unit having an acid-labile group (B).
   [8]: The chemically-amplified resist composition of the above [6] or [7], wherein the repeating unit having an acid-labile group is represented by the following general formula (B1),

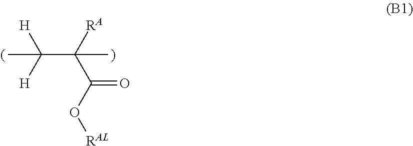

wherein R^{A} represents a hydrogen atom or a methyl group, and R^{AL} represents an acid-labile group.
   [9]: The chemically-amplified resist composition of any one of the above [6] to [8], wherein the base polymer has at least one selected from the group consisting of a repeating unit represented by the following general formula (B2), a repeating unit represented by the following general formula (B3), and a repeating unit represented by the following general formula (B4),

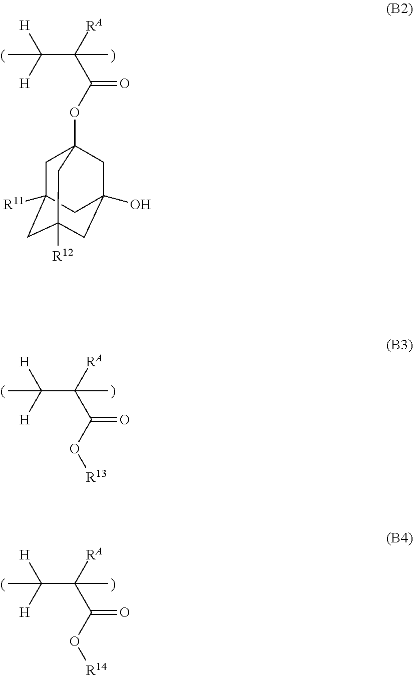

wherein
R^{A} represents a hydrogen atom or a methyl group;
R¹¹ and R¹² each independently represent a hydrogen atom or a hydroxy group;
R¹³ represents a substituent having a lactone structure or a substituent having a sultone structure; and
R¹⁴ represents a hydrogen atom, a fluorinated hydrocarbyl group having 1 to 15 carbon atoms, or a monovalent fluoroalcohol-containing substituent having 1 to 15 carbon atoms.
   [10]: The chemically-amplified resist composition of any one of the above [6] to [9], further comprising an organic solvent (C).
   [11]: The chemically-amplified resist composition of any one of the above [6] to [10], further comprising a fluorine-atom-containing polymer (D) having at least one selected from the group consisting of a repeating unit represented by the following general formula (D1), a repeating unit represented by the following general formula (D2), a repeating unit represented by the following general formula (D3), and a repeating unit represented by the following general formula (D4),

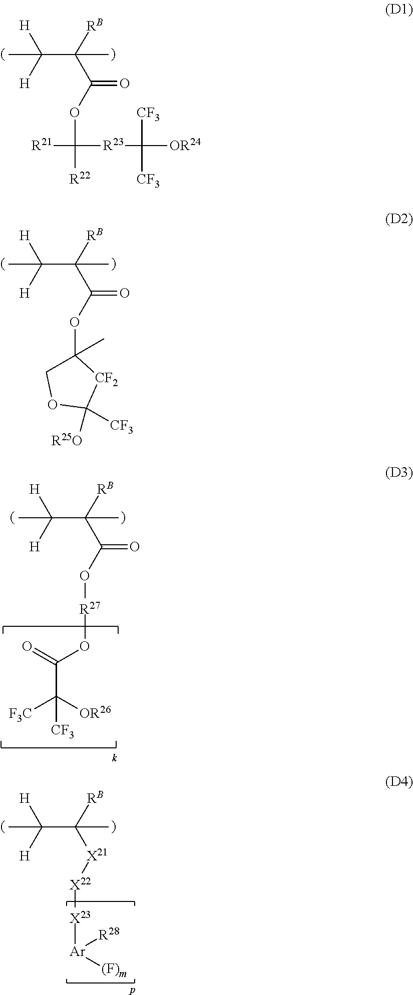

wherein
R^{B} represents a hydrogen atom or a methyl group;
R²¹ and R²² each independently represent a hydrogen atom or a hydrocarbyl group having 1 to 10 carbon atoms;
R²³ represents a single bond or a linear or branched hydrocarbylene group having 1 to 5 carbon atoms;
R²⁴, R²⁵, and R²⁶ each independently represent a hydrogen atom, a hydrocarbyl group having 1 to 15 carbon atoms, a fluorinated hydrocarbyl group having 1 to 15 carbon atoms, an acyl group having 2 to 15 carbon atoms, or an acid-labile group, when R²⁴, R²⁵, and R²⁶ are a hydrocarbyl group or a fluorinated hydrocarbyl group, part of —CH₂— thereof may be substituted with an ether bond or a carbonyl group;
R²⁷ represents a (k+1)-valent hydrocarbon group having 1 to 20 carbon atoms or a (k+1)-valent fluorinated hydrocarbon group having 1 to 20 carbon atoms;
“k” represents an integer of 1 to 3;
X²¹ each independently represents a single bond, an ester bond, an ether bond, an amide bond, or a phenylene bond;
“p” represents an integer of 1 or 2;
X²² each independently represents a single bond or a hydrocarbylene group having 1 to 20 carbon atoms when “p” is 1, X²² each independently represents a trivalent hydrocarbon group having 1 to 20 carbon atoms when “p” is 2, and the hydrocarbylene group and the trivalent hydrocarbon group may have at least one selected from an oxygen atom, a sulfur atom, a nitrogen atom, and a halogen atom;
X²³ each independently represents a single bond, an ester bond, an ether bond, an amide bond, a sulfonic acid ester bond, a urethane bond, a thiourethane bond, or a urea bond; Ar represents a group derived from benzene or naphthalene;
R²⁸ each independently represents a hydrogen atom, a saturated hydrocarbyl group having 1 to 6 carbon atoms, a saturated hydrocarbyloxy group having 1 to 6 carbon atoms, a saturated hydrocarbyloxycarbonyl group having 2 to 6 carbon atoms, a hydroxy group, a carboxy group, a halogen atom, a cyano group, or a nitro group; and
“m” represents an integer of 1 to 5.
   [12]: The chemically-amplified resist composition of any one of the above [6] to [11], further comprising a quencher (E).
   [13]: The chemically-amplified resist composition of any of the above [6] to [12], further comprising a surfactant (G).
   [14]: A patterning process comprising the steps of:
forming a resist film on a substrate by using the chemically-amplified resist composition of any of the above [6] to [13];
exposing the resist film by using a high-energy radiation; and
developing the exposed resist film by using a developer.
   [15]: The patterning process of the above [14], wherein KrF excimer laser light, ArF excimer laser light, an electron beam, or an extreme ultraviolet ray having a wavelength of 3 to 15 nm is used as the high-energy radiation.

It should be noted that the present invention is not limited to the above-described embodiments. The embodiments are just examples, and any examples that have substantially the same feature and demonstrate the same functions and effects as those in the technical concept disclosed in claims of the present invention are included in the technical scope of the present invention.

1. An onium salt represented by the following general formula (a), 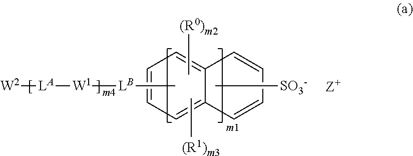
wherein
“m1” represents 0 to 1; “m2” represents an integer of 1 to 4 when “m1” is 0, “m2” represents an integer of 1 to 6 when “m1” is 1, “m3” represents an integer of 0 to 3 when “m1” is 0, “m3” represents an integer of 0 to 5 when “m1” is 1, provided that 2≤m2+m3≤4 when “m1” is 0 and 2≤m2+m3≤6 when “m1” is 1; and “m4” represents an integer of 0 to 2;
R⁰ represents an electron-withdrawing group other than a fluorine atom, and R⁰s may be the same as or different from each other when “m2” is 2 or more;
R¹ represents a fluorine atom, a hydroxy group, or a hydrocarbyl group having 1 to 20 carbon atoms and optionally having a heteroatom, R¹s may be the same as or different from each other when “m3” is 2 or more, and furthermore multiple R¹s may be bonded to each other to form a ring together with the carbon atoms to which they are bonded when “m3” is 2 or more;
L^{A} and L^{B} each independently represent a single bond, an ether bond, an ester bond, an amide bond, a sulfonate ester bond, a sulfonamide bond, a carbonate bond, or a carbamate bond;
W¹ represents a hydrocarbylene group having 1 to 40 carbon atoms, optionally having a heteroatom, and not involving resonance structures; W² represents a hydrocarbyl group having 1 to 40 carbon atoms, optionally having a heteroatom, and not involving resonance structures; and
Z⁺ represents a sulfonium cation represented by the following general formula (Z-1) or an iodonium cation represented by the following general formula (Z-2), 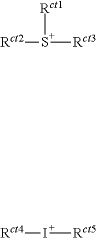
wherein
R^{ct1} to R^{ct5} each independently represent a halogen atom or a hydrocarbyl group having 1 to 30 carbon atoms and optionally having a heteroatom, and R^{ct1} and R^{ct2} may be bonded to each other to form a ring together with the sulfur atom to which they are bonded.

2. The onium salt according to claim 1, wherein at least one of W¹ and W² in the general formula (a) have an alicyclic structure having 3 to 40 carbon atoms and optionally having a hetero atom.

3. The onium salt according to claim 1, represented by the following general formula (a1), 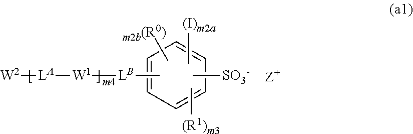
wherein “m3”, “m4”, L^{A}, L^{B}, R⁰, R¹, W¹, W², and Z⁺ represent the same as defined above, “m2a” represents an integer of 1 to 4, and “m2b” represents an integer of 0 to 3.

4. The onium salt according to claim 1, represented by the following general formula (a2), 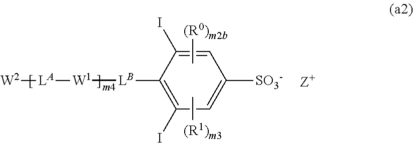
wherein “m4”, L^{A}, L^{B}, R⁰, R¹, W¹, W², and Z⁺ represent the same as defined above, and “m2b” and “m3” represent an integer of 0 to 2.

5. A photoacid generator comprising the onium salt according to claim 1.

6. A photoacid generator comprising the onium salt according to claim 2.

7. A photoacid generator comprising the onium salt according to claim 3.

8. A photoacid generator comprising the onium salt according to claim 4.

9. A chemically-amplified resist composition comprising the photoacid generator (A) according to claim 5.

10. The chemically-amplified resist composition according to claim 9, further comprising a base polymer having a repeating unit having an acid-labile group (B).

11. The chemically-amplified resist composition according to claim 10, wherein the repeating unit having an acid-labile group is represented by the following general formula (B1), 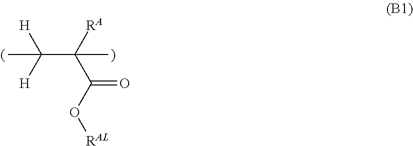
wherein R^{A} represents a hydrogen atom or a methyl group, and R^{AL} represents an acid-labile group.

12. The chemically-amplified resist composition according to claim 10, wherein the base polymer has at least one selected from the group consisting of a repeating unit represented by the following general formula (B2), a repeating unit represented by the following general formula (B3), and a repeating unit represented by the following general formula (B4), 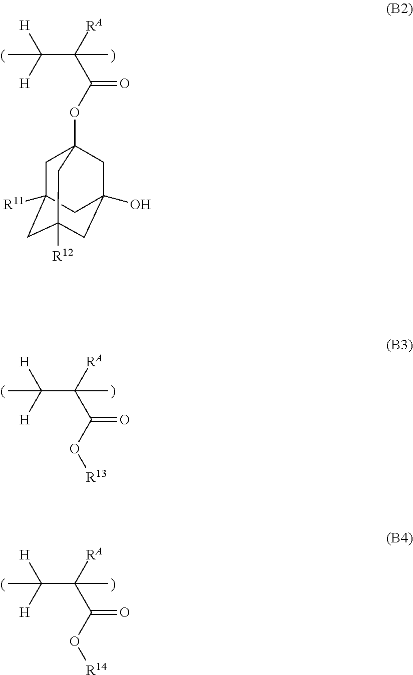
wherein
R^{A} represents a hydrogen atom or a methyl group;
R¹¹ and R¹² each independently represent a hydrogen atom or a hydroxy group;
R¹³ represents a substituent having a lactone structure or a substituent having a sultone structure; and
R¹⁴ represents a hydrogen atom, a fluorinated hydrocarbyl group having 1 to 15 carbon atoms, or a monovalent fluoroalcohol-containing substituent having 1 to 15 carbon atoms.

13. The chemically-amplified resist composition according to claim 9, further comprising an organic solvent (C).

14. The chemically-amplified resist composition according to claim 9, further comprising a fluorine-atom-containing polymer (D) having at least one selected from the group consisting of a repeating unit represented by the following general formula (D1), a repeating unit represented by the following general formula (D2), a repeating unit represented by the following general formula (D3), and a repeating unit represented by the following general formula (D4), 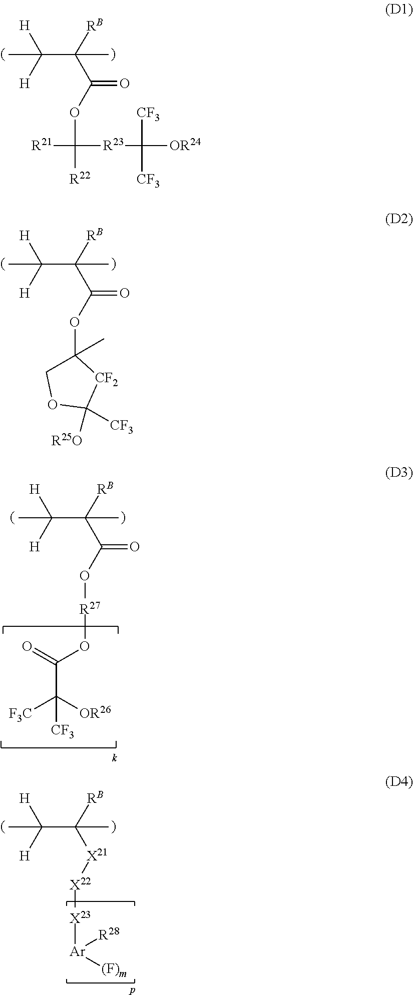
wherein
R^{B} represents a hydrogen atom or a methyl group;
R²¹ and R²² each independently represent a hydrogen atom or a hydrocarbyl group having 1 to 10 carbon atoms;
R²³ represents a single bond or a linear or branched hydrocarbylene group having 1 to 5 carbon atoms;
R²⁴, R²⁵, and R²⁶ each independently represent a hydrogen atom, a hydrocarbyl group having 1 to 15 carbon atoms, a fluorinated hydrocarbyl group having 1 to 15 carbon atoms, an acyl group having 2 to 15 carbon atoms, or an acid-labile group, when R²⁴, R²⁵, and R²⁶ are a hydrocarbyl group or a fluorinated hydrocarbyl group, part of —CH₂— thereof may be substituted with an ether bond or a carbonyl group;
R²⁷ represents a (k+1)-valent hydrocarbon group having 1 to 20 carbon atoms or a (k+1)-valent fluorinated hydrocarbon group having 1 to 20 carbon atoms;
“k” represents an integer of 1 to 3;
X²¹ each independently represents a single bond, an ester bond, an ether bond, an amide bond, or a phenylene bond;
“p” represents an integer of 1 or 2;
X²² each independently represents a single bond or a hydrocarbylene group having 1 to 20 carbon atoms when “p” is 1, X²² each independently represents a trivalent hydrocarbon group having 1 to 20 carbon atoms when “p” is 2, and the hydrocarbylene group and the trivalent hydrocarbon group may have at least one selected from an oxygen atom, a sulfur atom, a nitrogen atom, and a halogen atom;
X²³ each independently represents a single bond, an ester bond, an ether bond, an amide bond, a sulfonic acid ester bond, a urethane bond, a thiourethane bond, or a urea bond; Ar represents a group derived from benzene or naphthalene;
R²⁸ each independently represents a hydrogen atom, a saturated hydrocarbyl group having 1 to 6 carbon atoms, a saturated hydrocarbyloxy group having 1 to 6 carbon atoms, a saturated hydrocarbyloxycarbonyl group having 2 to 6 carbon atoms, a hydroxy group, a carboxy group, a halogen atom, a cyano group, or a nitro group; and
“m” represents an integer of 1 to 5.

15. The chemically-amplified resist composition according to claim 9, further comprising a quencher (E).

16. The chemically-amplified resist composition according to claim 9, further comprising a surfactant (G).

17. A patterning process comprising the steps of:
forming a resist film on a substrate by using the chemically-amplified resist composition according to claim 9;
exposing the resist film by using a high-energy radiation; and
developing the exposed resist film by using a developer.

18. The patterning process according to claim 17, wherein KrF excimer laser light, ArF excimer laser light, an electron beam, or an extreme ultraviolet ray having a wavelength of 3 to 15 nm is used as the high-energy radiation.
